SEQUENCE LISTING (1 sequence)

SEQ ID NO: 1 - protein, 191 aa, Homo sapiens [sequence omitted]

(19) United States
(12) Patent Application Publication
(10) Pub. No.: US 2013/0143815 A1
(43) Pub. Date: Jun. 6, 2013

(54) GROWTH HORMONE CONJUGATES

(71) Applicants: Henrik Sune Andersen, Holte (DK); Kjeld Madsen, Vaerloese (DK); Helle Demuth, Rungsted Kyst (DK); Christine Bruun Schioedt, Broenshoej (DK); Leif Noerskov-Lauritsen, Tappernoeje (DK); Peter Thygesen, Copenhagen OE (DK); Carsten Behrens, Copenhagen N (DK); Mette Guldbrandt, Farum (DK)

(72) Inventors: Henrik Sune Andersen, Holte (DK); Kjeld Madsen, Vaerloese (DK); Helle Demuth, Rungsted Kyst (DK); Christine Bruun Schioedt, Broenshoej (DK); Leif Noerskov-Lauritsen, Tappernoeje (DK); Peter Thygesen, Copenhagen OE (DK); Carsten Behrens, Copenhagen N (DK); Mette Guldbrandt, Farum (DK)

(73) Assignee: NOVO NORDISK HEALTH CARE AG, Zurich (CH)

(21) Appl. No.: 13/809,681

(22) PCT Filed: Jul. 15, 2011
(86) PCT No.: PCT/EP11/62152

§ 371 Date: Feb. 18, 2013

Related U.S. Application Data
(60) Provisional application No. 61/367,704, filed on Jul. 26, 2010.

(30) Foreign Application Priority Data
Jul. 22, 2010 (EP) 10170439.3

(51) Int. Cl. C07K 14/61

(52) U.S. Cl. CPC C07K 14/61
USPC 514/11.3; 530/399

(57) ABSTRACT

The present invention relates to growth hormone conjugates comprising a bile acid residue, said conjugation may occur through wt or mutant amino acid residues. The growth hormone polypeptide may be wt human growth hormone or a growth hormone variant.

### FIELD OF THE INVENTION

The present invention relates to growth hormone conjugates comprising a bile acid derivative. The conjugates are useful in treatment of growth hormone deficiencies or other treatments where human growth hormone is usually applied.

### BACKGROUND OF THE INVENTION

Growth hormone (GH) is a polypeptide hormone secreted by the anterior pituitary in mammals. Dependent on species GH is a protein composed of approximately 190 amino acid residues corresponding to a molecular weight of approximately 22 kDa. GH binds to and signals through cell surface receptors, the GH receptors (GHR). GH plays a key role in promoting growth, maintaining normal body composition, anabolism and lipid metabolism. It also has direct effects on intermediate metabolism, such as decreased glucose uptake, increased lipolysis, amino acid uptake and protein synthesis. The hormone also exerts effects on other tissues including adipose tissue, liver, intestine, kidney, skeleton, connective tissue and muscle. Recombinant hGH has been produced and commercially available as, for ex: Genotropin™ (Pharmacia Upjohn), Nutropin™ and Protropin™ (Genentech), Humatrope™ (Eli Lilly), Serostim™ (Serono), Norditropin (Novo Nordisk), Omnitrope (Sandoz), Nutropin Depot (Genentech and Alkermes). Additionally, an analogue with an additional methionine residue at the N-terminal end is also marketed as, fx: Somatonorm™ (Pharmacia Upjohn/Pfizer).

GH shares a common topology with the other members of the GH-family of proteins, Prolactin (PRL) and Placental Lactogen (PL). GH is classified as a four-helix bundle protein (FIG. 1) exhibiting an “up-up-down-down” topology with two conserved disulphide linkages. Specifically, wild-type human GH (hGH) is composed of 191 amino acid residues and has four cysteine residues at positions 53, 165, 182 and 189, which stabilizes the three dimensional structure of the protein by forming two intramolecular disulphide bonds connecting C53 with C165 and C182 with C189, respectively (FIG. 1). The structure of hGH has been experimentally determined by X-ray crystallography in the free form (Chantalet L. et al Protein and Peptide Letters (1995) 3, 333-340) and in complex with its binding protein (the extra cellular domain of the human GHR (hGHR)) (Devos, A. M. et al. Science (1992) 255, 306-312). These structures have been deposited in the Protein Data Bank (PDB) and are publicly available (PDB accession codes 1 HGU and 1HWG, respectively). Thus, from the published hGH structures residues important for hGH binding to hGHR can be identified. Furthermore, the dynamic properties of hGH has been studied by Nuclear Magnetic Resonance (NMR) spectroscopy (Kasimova M. R. et al. J. Mol. Biol. (2002) 318, 679-695). In combination, the X-ray and NMR data can distinguish regions of hGH which are well structured and well defined from regions which are less structured and dynamic. Less structured and dynamic regions of hGH are expected to be particularly susceptible to proteolytic cleavage and proper stabilization of such regions would lead to improved proteolytic stability.

hGH has been subject to extensive mutagenesis in attempts to produce hGH analogues with desired chemical or biological properties. Specifically, cysteine mutants for several purposes have been described.

US 2003/0162949 disclose cysteine variants of members of the GH supergene family. A general method is provided for creating site-specific, biologically active conjugates of these proteins. The method involves adding cysteine residues to non-essential regions of the proteins or substituting cysteine residues for non-essential amino acids in the proteins using site-directed mutagenesis and then covalently coupling a cysteine-reactive polymer or other type of cysteine-reactive moiety to the proteins via the added cysteine residue

WO 02/055532 describes genetically engineered hGH mutants having at least one non-polypeptide moiety covalently attached, particularly hGH mutants where a introduced cysteine residue was used for pegylation.

U.S. Pat. No. 5,951,972 describes physiologically active derivatized natural and recombinant mammalian and human proteins and polypeptides wherein at least one-naturally-occurring or incorporated cysteine residue within the protein is derivatized with various substituents.

The proteolytic cleavage of hGH has been studied in detail. The long loop composed of residues 128 to 154 has putative cleavage sites for several proteases, such as thrombin, plasmin, collagenase, subtilisin and chymotrypsin-like serine proteases. Accordingly, this part of hGH has been shown to be particularly susceptible to proteolytic cleavage (Lewis, U. J. Ann. Rev. Physiol. (1984) 46, 33-42). Enzymes reported to degrade hGH include thrombin, plasmin, subtilisin, chymotrypsin-like serine proteinases and kallikreins.

The degradation of hGH in rat tissue has been investigated (Garcia-Barros et al. J. Endocrinol. Invest. (2000) 23, 748-754).

In rat thyroid gland chymotrypsin-like proteases, favoring cleavage at bulky and lipophilic amino acid residues, were found initially to cleave the peptide bond between Y143 and S144 resulting in a two chain molecule, followed by cleavage between Y42 and S43, liberating the N-terminal peptide F1-Y42. The split loop in the two chain molecule is processed further by cleavage between F146 and D147 by chymotrypsin-like proteases and further by the action of carboxypeptidases.

Several methods to produce hGH analogues stabilized towards proteolytic degradation have been reported.

Alam et al., J. Biotech. (1998) 65, 183-190 designed hGH mutants resistant to thrombin and plasmin by specific point mutations. Thrombin cleaves hGH specifically between R134 and T135, and the double mutant R134D, T135P yielded a hGH variant resistant to cleavage by thrombin, and the triple mutant R134D, T135P, K140A resulted in resistance to plasmin. Furthermore, the latter hGH mutant was resistant to proteolysis by human plasma over a period of 7 days.

EP 534568 describes hGH mutants stabilized towards proteolytic degradation by mutating R134 to alanine, leucine, threonine, phenylalanine, proline or histidine.

WO 2004/022593/Nautilus describes general high through-put directed evolution methods to produce modified cytokines, including GH variants, with increased proteolytic stability.

WO 2006/048777/Nautilus specifically describes modified hGH analogues with improved proteolytic stability. The analogues contain one to five mutations at positions 1-55, 57, 58, 60-63, 67-87, 89-91, 93, 95-100, 102-128, 131-132, 135-139, 141, 142, 144, 148-182, 184, 185 and 187-191. Introduction of cysteine residues can potentially lead to the formation of undesired disulfide linked dimers and in WO 2006/048777 the substitution of amino acid residues by cysteine is specifically excluded from the scope; in WO 2006/048777 (p. 65) it is stated: “The replacement of amino acids by cysteine residues is explicitly avoided since this change would potentially lead to the formation of intermolecular disulfide bonds”.

There is an obvious need to develop hGH compounds which are resistant to proteolytic degradation. Such stabilized compounds should exhibit increased stability towards proteolytic cleavage while retaining the desired biological properties of hGH. Such GH molecules would have increased stability, slower clearance and/or prolong in vivo half-life.

Furthermore protein therapeutics generally needs to be administered intravenously or subcutaneously because they are generally not sufficiently orally available. The low oral bioavailability of proteins is partly due to proteolytic degradation in the gastrointestinal tract. Hence, there is also a need to develop hGH compounds that can be administered orally to treat hGH related disorders.

Naturally occurring bile acids are tightly constrained within the enterohepatic loop through the action of a series of transporter proteins (Carey, M. C.; Cahalane, M. J. Enterohepatic Circulation. In The Liver: Biology and Pathobiology, 2nd ed.; Raven Press: Ltd, New York, 1988; Chapter 33, pp 573-616). This enteric and hepatic uptake mechanisms ensure that about 90% of bile acids excreted into the intestine is reabsorbed in the ileum and recycled in what is referred to as the enterohepatic circulation, transferring the bile salts from the intestinal system back to the liver and the gallbladder.

A growth hormone compound or conjugate that poses properties similar to naturally occurring bile acids may show oral bioavailability as well as pharmacological relevant hepatic levels and a high liver-to-plasma (or -tissue) ratio and is therefore an high attractive compound.

### SUMMARY OF THE INVENTION

The present invention describes growth hormone conjugates having a bile acid covalently bond. The bile acid may as described herein be attached to various growth hormone compounds, such as protease stabilized growth hormone variant.

The invention in an aspect relates to a growth hormone conjugate which include a bile acid residue linked to a growth hormone compound. Such growth hormone conjugate may be described by formula (I)

A-W—B-GH (I)

wherein
GH represents a growth hormone compound,
A represents a bile acid residue,
B represents a hydrophilic spacer covalently linked to GH,
W is a chemical group linking A and B;
and pharmaceutically acceptable salt thereof.

In further embodiments of the invention the growth hormone compound (GH) is a human growth hormone (SEQ ID NO 1) or a variant thereof, having one or more amino acid substitutions.

The invention in further aspects relate to any use of such growth hormone conjugates, such as it's use in methods of treatment of growth hormone deficiency.

The invention in a further aspect relates to a method of preparing a growth hormone conjugate which include a bile acid residue linked to a growth hormone compound, wherein said bile acid residue is covalently linked to the growth hormone compound.

### DESCRIPTION OF THE DRAWINGS

FIG. 1

Structure of hGH bound to two copies of the hGH binding protein (PDB 1HWG). The four major helices in hGH are shown in dark gray and are labelled H1-H4. The directions (N→C terminal) are indicated by arrows. The N- and C-termini of hGH are labeled N and C, respectively. The two disulphide bonds connecting C53 with C165 and C182 with C189, respectively, are represented by black sticks and balls. Also labelled are L128 and D154 representing the first and last residues, respectively, in the long flexible loop connecting H3 and H4.

FIG. 2

Wild type amino acid sequence of hGH with the four main helices (H1-H4) highlighted and labelled. Also labelled are the three loops (L1-L3) connecting the main helices. The helix definitions refer to hGH in complex with its binding protein (PDB 1HWG).

### DEFINITIONS

In the present context, the terms “peptide” and “polypeptide” are used interchangeably and are intended to indicate the same. The terms “peptide” or “polypeptide” are intended to indicate a sequence of two or more amino acids joined by peptide bonds, wherein said amino acids may be natural or unnatural. The constituent amino acids may be from the group of the amino acids encoded by the genetic code and they may be natural amino acids which are not encoded by the genetic code, as well as synthetic amino acids. Natural amino acids which are not encoded by the genetic code are e.g. Hyp (hydroxyproline), γ-carboxyglutamate, Orn (ornithine), phosphoserine, D-alanine and D-glutamine. Synthetic amino acids comprise amino acids manufactured by chemical synthesis, such as D-isomers of the amino acids encoded by the genetic code such as D-alanine and D-leucine, Aad (α-aminoadipic acid), Aib (α-aminoisobutyric acid), Abu (α-aminobutyric acid), Agl (α-amino-glycine), Asu (α-aminosuberic acid), Cha (β-cyclopentyl-alanine), Chg (cyclohexyl glycine), Dab (α,γ-diaminobutyric acid), Dap (α,β-diaminopropanic acid), Hcy (homocysteine), Hpr (homoproline), Nle (Norleucine), Phg (phenylglycine), Hph (homophenylalanine), 1Nal (β-(1-naphthyl-alanine), 2Nal (β-(2-naphthyl-alanine), 2 Pal (-(2-pyridyl)-alanine, 3 Pal (β-(3-pyridyl)-alanine), Pip (4-amino-piperidine-4-carboxylic acid), Pra (propargyl-glycine), Pyr (pyroglutamic acid), Gla (γ-carboxy-glutamic acid), Hci (homocitruline), Nva (norvaline), Tle (tert-butylglycine), β-alanine, 3-aminomethyl benzoic acid and anthranilic acid.

The term also encompasses the term “proteins”, which may consists of one polypeptide chain, or two or more polypeptide chains held together by non-covalent or covalent interactions, such as for instance cysteine disulfide bridges.

It is to be understood that the term is also intended to include peptides, which have been derivatized, for instance by attaching moieties such as, but not limited to, PEG, carbohydrates, fatty acids, albumin binders, alkyl chains, lipophilic groups, vitamins, bile acids, or spacers to the side chains or main chain of the peptide in addition to comprising the additional disulfide bonds. The term peptide includes any suitable peptide and may be used synonymously with the terms polypeptide and protein, unless otherwise stated or contradicted by context, provided that the reader recognize that each type of respective amino acid polymer-containing molecule may be associated with significant differences and thereby form individual embodiments of the present invention (for example, a peptide such as an antibody, which is composed of multiple polypeptide chains, is significantly different from, for example, a single chain antibody, a peptide immunoadhesin, or single chain immunogenic peptide). Therefore, the term peptide herein should generally be understood as referring to any suitable peptide of any suitable size and composition (with respect to the number of amino acids and number of associated chains in a protein molecule). Moreover, peptides described herein may comprise non-naturally occurring and/or non-L amino acid residues, unless otherwise stated or contradicted by context.

The term peptide, unless otherwise stated or contradicted by context, (and if discussed as individual embodiments of the term(s) polypeptide and/or protein) also encompasses derivatized peptide molecules. A derivatized peptide molecules is one in which one or more of the amino acid residues of the peptide have been chemically modified (for instance by alkylation, acylation, ester formation, or amide formation) or associated with one or more non-amino acid organic and/or inorganic atomic or molecular substituents (for instance a polyethylene glycol (PEG) group, a lipophilic substituent (which optionally may be linked to the amino acid sequence of the peptide by a spacer residue or group such as β-alanine, γ-aminobutyric acid (GABA), L/D-glutamic acid, succinic acid, and the like), a fluorophore, biotin, a radionuclide, etc.) and may also or alternatively comprise non-essential, non-naturally occurring, and/or non-L amino acid residues, unless otherwise stated or contradicted by context (however, it should again be recognized that such derivatives may, in and of themselves, be considered independent features of the present invention and inclusion of such molecules within the meaning of peptide is done for the sake of convenience in describing the present invention rather than to imply any sort of equivalence between naked peptides and such derivatives).

Non-limiting examples of such amino acid residues include for instance 2-aminoadipic acid, 3-aminoadipic acid, β-alanine, β-aminopropionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, 2,4-diaminobutyric acid, desmosine, 2,2′-diaminopimelic acid, 2,3-diaminopropionic acid, N-ethylglycine, N-ethylasparagine, hydroxylysine, allohydroxylysine, 3-hydroxyproline, 4-hydroxyproline, isodesmosine, alloisoleucine, N-methylglycine, N-methylisoleucine, 6-N-methyllysine, N-methylvaline, norvaline, norleucine, ornithine, propargyl-glycine and statine halogenated amino acids.

A “compound” described in the present invention may be a “protein” or “peptide” or “polypeptide” which may be an “analogue” or a “variant”, which retains desired biological activities similar to wthGH, irrespective to the manner it has been modified.

The term “analogue” or “variant” as used herein when referring to a polypeptide, means a modified version of said peptide wherein one or more amino acid residues of the peptide have been substituted by other amino acid residues and/or wherein one or more amino acid residues have been deleted from the peptide and or wherein one or more amino acid residues have been added to the peptide. Such substitution or addition or deletion of amino acid residues can take place at the N-terminal of the peptide and/or at the C-terminal of the peptide and/or in between N- or C-terminal of the peptide. All amino acids for which the optical isomer is not stated are to be understood to mean the L-isomer.

The terms “disulphide bond” or “disulphide bridge” are used interchangeably and intended to indicate the same. A “disulphide bond” or “disulphide bridge” in proteins is formed between the thiol groups of cysteine residues.

The term “additional cysteine” or “introduced cysteine” are used interchangeably and are intended to indicate the same. The terms are intended to include a cysteine residue not present in wild type hGH. To minimize structural changes the cysteine residue(s) are usually introduces by substitution of amino acid residue(s), whereby the length of hGH is maintained. Insertion of an additional cys residue may be tolerated in loop sections or at the boarders of the helixes, whereas introduction of cys residues within the helix'es is less attractive.

The term “additional disulphide bond” or “introduced disulphide bond” are used interchangeably and are intended to indicate the same. The terms are intended to include disulphide bonds formed between two cysteine residues of which at least one is not present in wild type hGH.

The term “single point mutation” is used herein to indicate a mutation (compared to hGH as defined in SEQ ID NO 1). The term “additional single point mutation” may be used to specify that a single point mutation is unrelated to any disulphide bridge forming additional cysteine mutations introduced into the growth hormone compound. The term “additional single cys mutation” may be used to specify a point mutation which introduces a cysteine residue. Such a mutation may be the “additional single point mutation” or an even further mutation in hGH.

The term “conjugate” as used herein for defining the invention indicates a peptide or polypeptide, wherein one or more amino acid residues of the peptide have been covalently linked to a bile acid residue. The term “conjugate” is thus for the present invention used as a term to distinguish bile acid conjugates from alternative derivatives. It is although noticed that other documents use the terms “conjugate” and “derivative” interchangeably, so when referring to other documents the terms may be use interchangeably. As a verb, the term is intended to indicate the process of bonding a moiety (here a bile acid residue or linker) to a protein or polypeptide.

The term “derivative” as used herein refers to a peptide or polypeptide, wherein one or more amino acid residues of the peptide have been chemically modified by introduction of a polymer such as PEG, carbohydrate moieties, albumin binders, fatty acids, lipophilic groups, vitamins or spacers to the side chains or main chain of the growth hormone compound. The chemical modifications may also be transient in nature, i.e. they may readily be removed in vivo. The chemical modifications can be post-translationally introduced, for instance by the cell itself or by chemical modifications performed on the peptide after expression.

In the present context, the words “human growth hormone (hGH)’, “hGH wt” and “wild type hGH (wthGH)” are used interchangeably and refer both to a protein with an amino acid sequence as SEQ ID No.1.

The term “identity” as known in the art, refers to a relationship between the sequences of two or more peptides, as determined by comparing the sequences. In the art, “identity” also means the degree of sequence relatedness between peptides, as determined by the number of matches between strings of two or more amino acid residues. “Identity” measures the percent of identical matches between two or more sequences with gap alignments (if any) addressed by a particular mathematical model or computer program (i.e., “algorithms”). Identity of related peptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. (1988) 48, 1073.

Preferred methods to determine identity are designed to give the largest match between the sequences tested. Methods to determine identity are described in publicly available computer programs. Preferred computer program methods to determine identity between two sequences include the GCG program package, including GAP (Devereux et al., Nucl. Acid. Res. (1984) 12, 387; Genetics Computer Group, University of Wisconsin, Madison, Wis.), BLASTP, BLASTN, and FASTA (Altschul et al., J. Mol. Biol. (1990) 215, 403-410). The BLASTX program is publicly available from the National Center for Biotechnology Information (NCBI) and other sources (BLAST Manual, Altschul et al. NCB/NLM/NIH Bethesda, Md. 20894; Altschul et al., supra). The well known Smith Waterman algorithm may also be used to determine identity.

For example, using the computer algorithm GAP (Genetics Computer Group, University of Wisconsin, Madison, Wis.), two peptides for which the percent sequence identity is to be determined are aligned for optimal matching of their respective amino acids (the “matched span”, as determined by the algorithm). A gap opening penalty (which is calculated as 3.times. the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see Dayhoff et al., Atlas of Protein Sequence and Structure, (1978) 5 for the PAM 250 comparison matrix; Henikoff et al., PNAS USA (1992) 89, 10915-10919 for the BLOSUM 62 comparison matrix) is also used by the algorithm.

Preferred parameters for a peptide sequence comparison include the following: Algorithm: Needleman et al., J. Mol. Biol. (1970) 48, 443-453; Comparison matrix: BLOSUM 62 from Henikoff et al., PNAS USA (1992) 89, 10915-10919; Gap Penalty: 12, Gap Length Penalty: 4, Threshold of Similarity:

The GAP program is useful with the above parameters. The aforementioned parameters are the default parameters for peptide comparisons (along with no penalty for end gaps) using the GAP algorithm.

The terms “protease or proteases” is intended to include all enzymes possessing the ability to catalyze hydrolytic cleavage of a peptide bond. Proteases may be intra cellular, extra cellular or membrane bound proteases, proteinases or peptidases, and include proteases in the lumen of mammalian intestine and proteases present in mammalian plasma. Proteases may both be of the type endo proteases and exo proteases. Proteases may be of, but are not limited to, the following types: serine, cysteine, aspartic or metallo proteases. Specific examples of proteases are Trypsin, Chymotrypsin, Pepsin, Elastase, Factor Vila, Factor Xa, Proteinase K, Carboxy peptidase, DPPIV, Neutral Endopeptidase, Granzyme B, Proline-endopeptidase, Thermolysin, Thrombin, Arg-C proteinase, Asp-N endopeptidase, Caspase 1-10, Clostripain, Enterokinase, Glutamyl endopeptidase, LysC, LysN and Staphylococcal peptidase I.

The terms “resistant to proteolytic degradation” or “increased stability towards proteolytic degradation” or “increased stability towards proteolytic cleavage” or “improved proteolytic stability” or “proteolytic stability” are used interchangeably and intended to indicate the same. Used in connection to a hGH compound of the invention, the terms are intended to indicate that the polypeptide chain of said hGH compound is cleaved at a slower rate, compared to wild type hGH, by a protease under specific conditions.

The rate of proteolytic cleavage of a protein may be measure by several techniques known to the person skilled in the art. An example of an assay measuring the rate of degradation of hGH or a hGH compound is described in Example 5.

The term “functional in vivo half-life” is used in its normal meaning, i.e., the time at which 50% of the biological activity of the peptide, for instance a growth hormone compound, wherein the growth hormone compound is still present in the body/target organ, or the time at which the activity of the peptide, for instance growth hormone compound is 50% of its initial value. As an alternative to determining functional in vivo half-life, “in vivo plasma half-life”, and protracted action may be determined, i.e., the time at which 50% of the peptide circulate in the bloodstream prior to being cleared. Determination of plasma half-life is often more simple than determining functional half-life and the magnitude of plasma half-life is usually a good indication of the magnitude of functional in vivo half-life.

The term “alkane” or “alkyl” is intended to indicate a saturated, linear, branched and/or cyclic hydrocarbon. Unless specified with another number of carbon atoms, the term is intended to indicate hydrocarbons with from 1 to 30 (both included) carbon atoms, such as 1 to 20 (both included), such as from 1 to 10 (both included), e.g. from 1 to 5 (both included). The terms alkyl and alkylene refer to the corresponding radical and bi-radical, respectively.

The term “C₁₋₆ alkyl” refers to a straight chained or branched saturated hydrocarbon having from one to six carbon atoms inclusive. Examples of such groups include, but are not limited to, methyl, 2-propyl, 1-butyl, 2-butyl, 2-methyl-2-propyl, 2-methyl-1-butyl and n-hexyl.

The term “C₃₋₁₀ cycloalkyl” typically refers to cyclopropyl, cyclobutyl, cyclopentyl, cyclohexyl, cycloheptyl, cyclooctyl, cyclononyl, and cyclodecanyl.

The term “alkene” is intended to indicate linear, branched and/or cyclic hydrocarbons comprising at least one carbon-carbon double bond. Unless specified with another number of carbon atoms, the term is intended to indicate hydrocarbons with from 2 to 30 (both included) carbon atoms, such as 2 to 20 (both included), such as from 2 to 10 (both included), e.g. from 2 to 5 (both included). The terms alkenyl and alkenylene refer to the corresponding radical and bi-radical, respectively.

The term “alkyne” is intended to indicate linear, branched and/or cyclic hydrocarbons comprising at least one carbon-carbon triple bond, and it may optionally comprise one or more carbon-carbon double bonds. Unless specified with another number of carbon atoms, the term is intended to indicate hydrocarbons with from 2 to 30 (both included) carbon atoms, such as from 2 to 20 (both included), such as from 2 to 10 (both included), e.g. from 2 to 5 (both included). The terms alkynyl and alkynylene refer to the corresponding radical and bi-radical, respectively.

The term “homocyclic aromatic compound” is intended to indicate aromatic hydrocarbons, such as benzene and naphthalene.

The term “heterocyclic compound” is intended to indicate a cyclic compound comprising 5, 6 or 7 ring atoms from which 1, 2, 3 or 4 are hetero atoms selected from N, O and/or S. Examples include heterocyclic aromatic compounds, such as thiophene, furan, pyran, pyrrole, imidazole, pyrazole, isothiazole, isooxazole, pyridine, pyrazine, pyrimidine, pyridazine, as well as their partly or fully hydrogenated equivalents, such as piperidine, pirazolidine, pyrrolidine, pyrroline, imidazolidine, imidazoline, piperazine and morpholine.

The terms “hetero alkane”, “hetero alkene” and “hetero alkyne” are intended to indicate alkanes, alkenes and alkynes as defined above, in which one or more hetero atom or group have been inserted into the structure of said moieties. Examples of hetero groups and atoms include —O—, —S—, —S(O)—, —S(O)₂—, —C(O)— —C(S)— and —N(R*)-, wherein R* represents hydrogen or C₁-C₆-alkyl. Examples of heteroalkenes include.

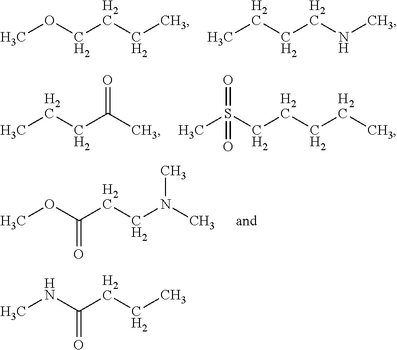

The term “radical” or “biradical” is intended to indicate a compound from which one or two, respectively, hydrogen atoms have been removed. When specifically stated, a radical may also indicate the moiety formed by the formal removal of a larger group of atoms, e.g. hydroxyl, from a compound.

The term “halogen” is intended to indicate members of the seventh main group of the periodic table, e.g. F, Cl, Br and I.

In the present context, the term “aryl” is intended to indicate a carbocyclic aromatic ring radical or a fused aromatic ring system radical wherein at least one of the rings are aromatic. Typical aryl groups include phenyl, biphenylyl, naphthyl, and the like.

The term “heteroaryl” or “hetaryl”, as used herein, alone or in combination, refers to an aromatic ring radical with for instance 5 to 7 member atoms, or to a fused aromatic ring system radical with for instance from 7 to 18 member atoms, wherein at least one ring is aromatic, containing one or more heteroatoms as ring atoms selected from nitrogen, oxygen, or sulfur heteroatoms, wherein N-oxides and sulfur monoxides and sulfur dioxides are permissible heteroaromatic substitutions. Examples include furanyl, thienyl, thiophenyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, quinolinyl, isoquinolinyl, benzofuranyl, benzothiophenyl, indolyl, and indazolyl, and the like.

The term “motif” is use to describe small chemical entities within a larger entity, such as the spacer (B), wherein such motifs may be formed by one or more chemical groups.

The term “residue” is used for a chemical unit, equal to the usual use in relation to amino acids, e.g. a protein or peptide consists of covalently linked amino acid residues. In particular the term residue is used in relation to the bile acid residue, which denotes the bile acid covalently bond to the spacer via a chemical linking group.

In the present context, the term “pharmaceutically acceptable salt” is intended to indicate salts which are not harmful to the patient. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, paminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 66, 2, (1977) which is incorporated herein by reference. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

### DESCRIPTION OF THE INVENTION

### Growth Hormone Conjugate

An aspect of the invention relates to growth hormone conjugates comprising a bile acid residue covalently linked to growth hormone compound. The conjugates may in one embodiment be described by formula (I)

A-W—B-GH (I)

wherein
GH represents a growth hormone compound,
A represents a bile acid residue,
B represents a hydrophilic spacer covalently linked to GH,
W is a chemical group linking A and B;
and pharmaceutically acceptable salt thereof.

As described herein below, the components of such conjugates can be varied by including different growth hormone variants or derivatives, providing additional functionalities while maintaining the stimulating effect on the growth hormone receptor.

Additionally, the major functionalities of human growth hormone are usually maintained in the growth hormone conjugate. The in vitro and in vivo activity of a human growth hormone conjugate may be test as any growth hormone compound.

### Growth Hormone Compound

According to the invention the growth hormone compound retain the major functionalities of human growth hormone and may in one embodiment be identical to human growth hormone (SEQ ID NO 1).

In one embodiment of the present invention a growth hormone compound is a polypeptide comprising an amino acid sequence having at least 20%, such as at least 30%, for instance at least 40%, such as at least 50%, for instance at least 60%, such as at least 70%, for instance at least 80%, such as at least 90% identity, for instance at least 95%, such as at least 96%, for instance at least 97%, such as at least 98% identity to SEQ ID No. 1.

In one embodiment the growth hormone compound has an in vitro activity which is comparable to the in vitro activity of hGH defined by SEQ ID NO 1. In vitro activity of growth hormone compounds is preferably measured in a BAF assay as described in Method 5 herein.

In further embodiments as described herein below the growth hormone compound has growth hormone activity of at least 1%, such as at least 5%, for instance at least 10%, such as at least 25% of the activity of hGH as determined in a BAF assay (described in Method 5 and/or as determined in hyposectomized rats (described in Method 6).

In one embodiment the compound may have an in vitro activity which is different from the in vitro activity of hGH. As described above a lower in vitro activity may be compensated by other functionalities.

In one embodiment the in vitro activity may be such as at least 1%, such as at least 5%, for instance at least 10%, such as at least 25% of the activity of hGH. In a further embodiment the EC₅₀ ratio for a compound relative to wild type hGH defined by SEQ ID No. 1 is not more that 10, not more than 8, not more than 6, not more than 4, not more than 2. In an embodiment the EC₅₀ ratio for said compound compared to wild type hGH defined by SEQ ID No. 1 is from 5-0.01 or such as from 3-0.01 or such as is from 2-0.01. In an alternative the EC₅₀ may according to the invention be measure by Surface Plasmon Resonance analysis (SPR) as described in Method 7. In corresponding embodiments the in vitro activity determined by SPR, may be such as at least 1%, such as at least 5%, for instance at least 10%, such as at least 25% of the activity of hGH. In further embodiments the EC₅₀ ratio for a compound relative to wild type hGH defined by SEQ ID No. 1 determined by SPR is not more that 10, not more than 8, not more than 6, not more than 4, not more than 2. In one embodiment the EC₅₀ ratio for said compound compared to wild type hGH defined by SEQ ID No. 1 is from 5-0.01 or such as from 3-0.01 or such as 2-0.01.

To avoid doubt, a growth hormone compound may also have a higher activity than hGH in these assays.

In one embodiment, where the growth hormone compound is derivatized in some way (additional to the bile acid conjugation), the activity of the growth hormone in relation to hGH may be measured on the un-derivatized growth hormone compound, as the derivatization may change the activity significantly. For instance in the case of a growth hormone compound derivatized with a property-modifying group that prolongs the functional in vivo half-life of the growth hormone compound, the activity of the derivatized growth hormone compound may be much lower than the activity of hGH, which decrease is counteracting by the prolonged residence time.

In an alternative embodiment the growth hormone activation may be measured on the derivatized growth hormone compound. And in further embodiments the growth hormone activity is measured on the growth hormone conjugate (including bile acid conjugation) and any additional property modifying chemical modification to ensure that such conjugation and/or modification does not interfere with receptor interaction. It should be noted that the test are to be compared to hGH on a molar basis or on the relative protein/peptide content e.g. the weight of any derivation or conjugation should not be included.

In one embodiment, the growth hormone compound is a fragment of such a polypeptide, which fragment has retained a significant amount of the growth hormone activity as described above.

In one embodiment the growth hormone compound is a truncated version of hGH, i.e. one or more amino acid residues have been deleted from the N- and/or C-termini corresponding to SEQ No. 1 wherein the said compound retain desired biological properties of wild type hGH.

In one embodiment the growth hormone compound has increased in vivo half life.

In one embodiment the growth hormone compound has increased shelf life.

In one embodiment the growth hormone compound may be a fusion protein.

In one embodiment of the present invention the growth hormone compound is chemically modified (additional to the bile acid conjugate) via attaching moieties such as, but not limited to, PEGs, carbohydrates, albumin binders, fatty acids, alkyl chains, lipophilic groups, vitamins or spacers to the side chains or main chain of the growth hormone compound.

In one embodiment the growth hormone compound is chemically modified in order to facilitate transport across the epithelia when compared to hGH or a hGH variant not chemically modified. The chemical modification may be such as the conjugation of a cholic acid residue as describe herein. Different assays such as a Caco-2 cell assay or a MDCK cell assay may be used to estimate transport of growth hormone compounds across a cell layer.

In one embodiment the growth hormone compound of the present invention is chemically modified in order to obtain a prolonged duration of in vivo action.

In one embodiment of the present invention a growth hormone compound is chemically modified in order to obtain a prolonged duration of functional in vivo half-life.

In one embodiment the chemical modifications of the growth hormone compound may be transient in nature, i.e. they may readily be removed in vivo.

In one embodiment the growth hormone compound modifications can take place at any amino acid residue not interfering with binding of the growth hormone compound to the human growth hormone receptor (hGHR).

In further embodiments as described below the conjugation may occur at different positions selected from wt hGH residues and from mutant residues.

In one embodiment the conjugation is attached via the N-terminal or the C-terminal.

In one embodiment the conjugation is attached via a Gln residue, such as Gln 40 or Gln 141.

In such further embodiments where either the bile acid linker (or an alternative derivative) is attached to GH via a mutant residue the GH of the conjugate may have a single Cys mutation selected from any one of a single Cys mutation in the N-terminal, H1, L1, H2, L2 or H3 regions of GH.

In further such embodiments, the single Cys mutation is positioned in the N-terminal, the mutation being such as any one of T3C, P5C, S7C, or in H1 (corresponding to AA 9-35), the mutation being such as any one of 11C, H18C, Q29C, E30C, E33C, A34C, Y35C or in L1 corresponding to AA 36-71, the mutation being such as any one of Q40C, K41C, Y42C, S55C, S57C, S62C or in H2, L2 or H3 (corresponding to AA 72-98, AA 99-106 and AA 107-127), the mutation being such as any one of E88C, Q91C, S95C, A98C, N99C, S100C, L101C, V102C, Y103C, D107C, S108C, D112C, Q122C and G126C of hGH (SEQ ID NO:1). If the single Cys mutation is present in an hGH variant the mutation is located in corresponding amino acid residues.

Further embodiments includes GH conjugates wherein the single Cys mutation in GH is selected from any one of: T3C, P5C, S7C, D11C, H18C, Q29C, E30C, E33C, A34C, Y35C, Q40C, S55C, S57C, S62C, E88C, Q91C, S95C, A98C, N99C, S100C, L101C, V102C, Y103C, D107C, S108C, D112C, Q122C and G126C of hGH (SEQ ID NO:1), such as any one of Q40C, S62C and L101C.

In even further embodiments the single Cys mutation is located within AA 93-106 in hGH or corresponding residues in hGH variants.

In further specified embodiments the single Cys mutation is located within L2, such as within AA 99-106 or AA 99-103 or corresponding residues.

In order to obtain a growth hormone conjugate with prolonged in vivo residence time it is attractive to optimise the stability of the growth hormone compound, and especially to apply growth hormone compounds which are less susceptible to protein degradation.

In one embodiment the growth hormone compound has increased protease stability.

In one embodiment the growth hormone compound has increased stability towards proteolytic cleavage.

In one embodiment the growth hormone compound has increased stability towards proteolytic degradation by a pancreatic protease.

In one embodiment the growth hormone compound has increased stability towards proteolytic degradation by proteases present in the gastrointestinal tract.

In one embodiment the growth hormone compound has increased stability towards proteolytic degradation by proteases present in mammalian plasma.

In one embodiment the growth hormone compound has one or more additional disulphide bond(s). The disulphide bonds are formed between pairs of cysteines of which one or both are introduced by point mutations in the wild type hGH sequence. The sites of mutetion are chosen such that the introduced cysteine residues are appropriately placed in the three dimensional structure of the folded protein to allow for the formation of a disulphide bond. If only one cysteine is introduced, its partner in forming a disulphide bond will include one of the four cysteine residue present in wild type hGH. The folded protein with the additional disulphide bond may be obtained by expressing the appropriate cysteine mutant of hGH in soluble form by a suitable host organism, or recovered from inclusion bodies using standard refolding conditions for growth hormone compounds, which are well known to those skilled in the art (Cabrita and Bottomley, Biotechnology Annual Review (2004) 10, 31-50). The identification of candidate positions for introduction of additional disulphide bonds can be aided by computational methods, e.g. using the experimentally determined three dimensional structure of hGH (PDB accession code 1HWG) in complex with two copies of its binding protein. Selection of appropriate positions for introduction of disulphide bond can be based distance and geometry criteria for disulphide bonds described in Dombkowski A., A., Bioinformatics (2003) 19, 1852-1853 and Petersen et al., Protein Eng. (1999) 12, 535-548.

The cysteine mutants are chosen such that the introduced disulphide bonds do not disrupt the native structure of the protein and have minimal negative impact on the desired biological activity associated with hGH. Thus, the compounds are constructed such that the introduced disulphide bonds do not impair interaction with hGHR. The regions in hGH important for receptor interaction have been identified from 1HWG. Thus, the selection of appropriate positions for introducing disulphide bonds, which are neutral with respect to biological activity, can be guided by analyzing the 1HWG structure.

The cysteine mutants may be chosen such that the introduced disulphide bonds provide increased stability towards proteolytic cleavage. The susceptibility of a protein to protease cleavage is defined in part by the primary amino acid sequence of said protein. Proteases may be relatively unspecific or may, with variable degree of selectivity, recognize specific motifs in the primary amino acid sequence. However, the three dimensional structure and dynamics of the protein molecule acting as a substrate strongly influence proteolytic stability. Highly flexible and dynamic loop structures are particularly vulnerable to protease catalyzed cleavage, whereas well structured regions are generally less so. Thus, protection against proteolytic cleavage can be obtained by stabilizing dynamic regions of a protein by introducing disulphide bonds.

For disulphide bridges between two cysteine residues, the cysteine residues may be introduced or substituted in any of the regions or positions as defined herein in order to facilitate formation of one or more additional disulphide bonds. Substitution and insertions of amino acid residues can be carried out by standard techniques known to a person skilled in the art.

In one embodiment the growth hormone compound comprises one or more additional disulfide bond(s) compared to hGH as defined by SEQ ID NO 1. As described herein above the polypeptide of a growth hormone compound according to the invention preferably has a high level of identity to human growth hormone identified by SEQ ID NO 1.

Accordingly, stable GH compounds made resistant to proteolytic degradation by introduction of one or more additional disulphide bonds in hGH as defined by SEQ ID No.1 is subject to conjugation of a bile acid residue as described herein. In one embodiment the growth hormone compound is stabilized towards proteolysis.

In one embodiment the increased proteolytic stability of growth hormone compound is achieved by introducing a disulphide bond between a loop segment and a helical structure.

In one embodiment the increased proteolytic stability of growth hormone compound is achieved by introducing a disulphide bond within a loop segment.

In one embodiment the increased proteolytic stability of growth hormone compound is achieved by introducing a disulphide bond between loop segments.

In one embodiment the increased proteolytic stability of growth hormone compound is achieved by introducing a disulphide bond between helices.

In one embodiment at least one of the introduced disulphide bonds links two cysteine residues of a growth hormone compound, wherein at least one of said cysteine residues is not present in wild type hGH.

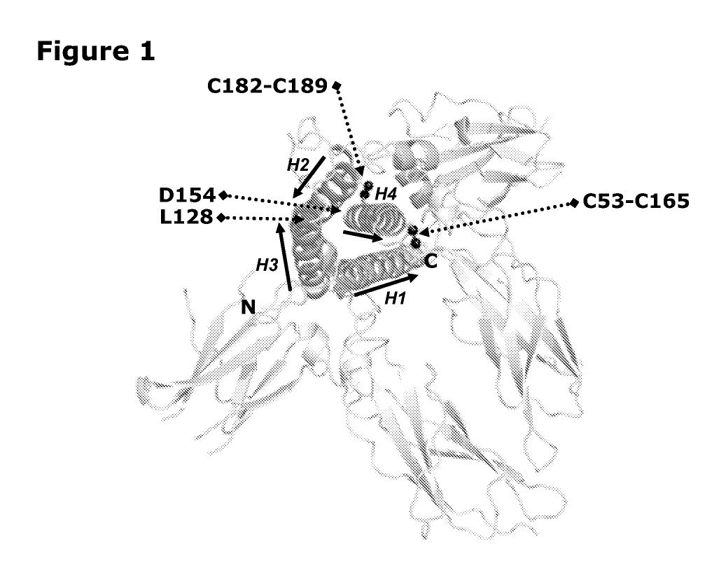

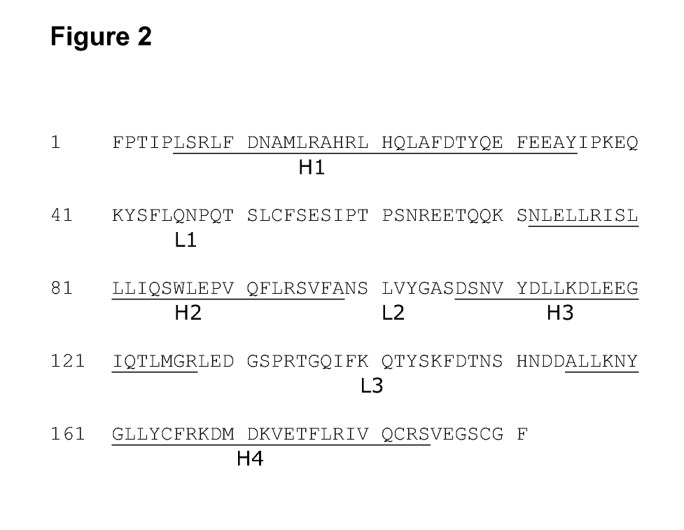

In one embodiment the introduced disulphide bond(s) of the growth hormone compound stabilize the loop connecting H3 and H4 (L3, residues 128-154), i.e. at least one of the cysteines in the introduced disulphide bond is positioned in the segment comprising residues 128-154 (FIGS. 1 and 2).

In one embodiment the introduced disulphide bonds of a growth hormone compound are positioned between cysteine residues that are selected using distance and geometry criteria described in Dombkowski A., A., Bioinformatics (2003) 19, 1852-1853 and Petersen et al., Protein Eng. (1999) 12, 535-548.

As described above the growth hormone compound comprising an additional disulfide bond connecting a loop segment and a helical segment or within a loop segment or connecting loop segments or connecting helical segments of the polypeptide. The location of any such additional disulfide bond is for the purpose of this application described with reference to the polypeptide of hGH as defined in SEQ ID NO 1. Non limiting examples of Cys mutations providing linkage connecting helix's, loops and a loop and a helix are listed in the table below.

| | First Amino Acid | Second Amino Acid | Secondary |
|---|---|---|---|
| | as defined by | as defined by | Structural |
| | sequence alignment | sequence alignment | segments |
| | with SEQ ID NO 1. | with SEQ ID NO 1. | connected^{a} |
| 1. | 16 | 117 | H1-H3 |
| 2. | 17 | 174 | H1-H4 |
| 3. | 21 | 170 | H1-H4 |
| 4. | 26 | 102 | H1-L2 |
| 5. | 26 | 103 | H1-L2 |
| 6. | 47 | 50 | L1-L1 |
| 7. | 49 | 161 | L1-L1 |
| 8. | 54 | 143 | L1-L3 |
| 9. | 54 | 144 | L1-L3 |
| 10. | 54 | 146 | L1-L3 |
| 11. | 55 | 143 | L1-L3 |
| 12. | 57 | 143 | L1-L3 |
| 13. | 58 | 141 | L1-L3 |
| 14. | 58 | 143 | L1-L3 |
| 15. | 58 | 144 | L1-L3 |
| 16. | 59 | 137 | L1-L3 |
| 17. | 61 | 66 | L1-L1 |
| 18. | 61 | 67 | L1-L1 |
| 19. | 71 | 132 | L1-L3 |
| 20. | 73 | 132 | H2-L3 |
| 21. | 73 | 139 | H2-L3 |
| 22. | 77 | 138 | H2-L3 |
| 23. | 77 | 139 | H2-L3 |
| 24. | 81 | 141 | H2-L3 |
| 25. | 81 | 143 | H2-L3 |
| 26. | 84 | 143 | H2-L3 |
| 27. | 84 | 144 | H2-L3 |
| 28. | 85 | 143 | H2-L3 |
| 29. | 85 | 144 | H2-L3 |
| 30. | 89 | 146 | H2-L3 |
| 31. | 92 | 146 | H2-L3 |
| 32. | 92 | 148 | H2-L3 |
| 33. | 94 | 107 | H2-H3 |
| 34. | 102 | 105 | L2-H3 |
| 35. | 156 | 146 | H4-L3 |
| 36. | 156 | 148 | H4-L3 |
| 37. | 185 | 188 | Ct-Ct |

^{a}H1-H4 refers to helix 1-4, L1-L3 refers to loops 1-3, and Ct refer to C-terminal segment.

In one embodiment the growth hormone compound comprises at least one pair of mutations corresponding to R16C/L117C, A17C/E174C, H21C/M170C, D26/V102C, D26/Y1030, N47C/T50C, Q49C/G1610, F54C/Y1430, F54C/S1440, F54C/F1460, S55C/Y1430, S57C/Y1430, 158C/Q1410, 158C/Y1430, 158C/S1440, P59C/Q1370, P61 C/E66C, P61 C/T67C, S71C/S1320, L73C/S1320, L73C/F1390, R77C/I138C, R77C/F1390, L81C/Q141C, L81C/Y143C, Q84C/Y1430, Q84C/S1440, S85C/Y1430, S85C/S1440, P89C/F1460, F92C/F1460, F92C/T1480, R94C/D107C, V102C/A1050, L156C/F146C, L156C/T148C and/or V185C/S188C in SEQ ID NO 1.

In one embodiment the growth hormone compound comprises at least one pair of mutations corresponding to A17C/E174C, H21C/M1700, D26/V1020, D26/Y103C, F54C/Y1430, F54C/S1440, F54C/F1460, S55C/Y1430, S57C/Y1430, 158C/Q1410, 158C/Y143C, 1580/S1440, P59C/Q137C, S71C/S132C, L81C/Y143C, Q84C/Y143C, S85C/Y1430, S85C/S144C, F92C/T1480 and/or R94C/D107C in SEQ ID NO 1.

One embodiment according to the invention the growth hormone compound of the conjugate comprises an additional disulfide bond wherein at least one of the cysteines is present in L3 (AA 128-154 in SEQ ID NO 1), or such as in the middle region of the loop defined by AA 135-148) or corresponding amino acid residues.

In one embodiment the growth hormone compound has at least one of the cysteines of the additional disulfide bond present in L3, in a position corresponding to AA 141, AA142, AA143, AA144, AA145 or AA146, preferably AA143 or AA144 in SEQ ID NO 1.

In one embodiment the growth hormone compound comprises at least one pair of mutations corresponding to F54C/Y143C, F54C/S144C, F54C/F146C, S55C/Y143C, S57C/Y143C, 158C/Q141C, 158C/Y143C, 158C/S144C, P59C/Q137C, S71C/S132C, L73C/S132C, L73C/F139C, R77C/1138C, R77C/F139C, L81C/Q141C, L81C/Y143C, Q84C/Y1430, Q84C/S144C, S85C/Y1430, S85C/S144C, P89C/F146C, F92C/F146C and/or F92C/T1480 in SEQ ID NO 1.

In one embodiment the growth hormone compound comprises at least one pair of mutations corresponding to F54C/Y143C, F54C/S144C, F54C/F146C, S55C/Y143C, S57C/Y143C, 158C/Q141C, 158C/Y143C, 158C/S144C, P59C/Q137C, S71C/S132C, L81C/Y143C, Q84C/Y143C, S85C/Y143C, S85C/S144C and/or F92C/T148C in SEQ ID NO 1.

One embodiment according to the invention relates to a conjugate where the growth hormone compound comprises an additional disulfide bond connecting L3 with L1.

In one embodiment the growth hormone compound comprises an additional disulfide bond connecting an amino acid residue corresponding to AA54, AA55, AA56, AA57, AA58 or AA59 in L3 with an amino acid corresponding to AA143 or AA144 in L1 of SEQ ID NO 1.

In one embodiment the growth hormone compound according to the invention comprises at least one pair of mutations corresponding to F54C/Y143C, F54C/S144C, F54C/F146C, S55C/Y143C, S57C/Y143C, 158C/Q141C, 158C/Y143C, 158C/S144C, P59C/Q137C and/or S71C/S132C in SEQ ID NO 1.

One embodiment according to the invention relates to a growth hormone compound comprising an additional disulfide bond connecting L3 with a helical segment, such as helix 2 (H2).

In one embodiment a growth hormone compound comprises an additional disulfide bond connecting an amino acid residue corresponding to AA84 or AA85 in H2 with an amino acid corresponding to AA143 or AA144 in L3 of SEQ ID NO 1.

In one embodiment a growth hormone compound comprises at least one pair of mutations corresponding to L73C/S132C, L73C/F139C, R77C/1138C, R77C/F139C, L81C/Q141C, L81C/Y143C, Q84C/Y143C, Q84C/S144C, S85C/Y143C, S85C/S144C, P89C/F146C, F92C/F146C and F92C/T148C in SEQ ID No.

In one embodiment a growth hormone compound comprises at least one pair of mutations corresponding to L81C/Y143C, Q84C/Y143C, S85C/Y143C, S85C/S144C and/or F92C/T148C in SEQ ID NO 1.

One embodiment according to the invention relates to a growth hormone compound comprising an additional disulfide bond connecting L2 with helix 1.

In one embodiment a growth hormone compound comprises at least one pair of mutations corresponding to D26C/V102C or D26C/Y103C.

In one embodiment the growth hormone compound comprising one or more additional disulfide bond(s) is stabilized towards degradation by protease(s), such as digestive proteases, such as pepsin, trypsin, chymotrypsin, carboxypeptidase and/or elastases.

In one embodiment of the present invention the growth hormone compound has increased stability towards proteolytic degradation by trypsin, chymotrypsin and/or elastase.

In one embodiment the growth hormone compound is stabilized towards degradation by chymotrypsin and/or elastase.

In one embodiment the growth hormone compound comprises at least one pair of mutations corresponding to H21/M170, D26/V102C, D26/Y103C, F54C/Y143C, F54C/S144C, S55C/Y143C, S57C/Y143C, 158C/Q141C, 158C/Y143C, 158C/S144C, P59C/Q137C, S71C/S132C, L81C/Y143C, Q84C/Y143C, S85C/Y143C and/or S85C/S144C in SEQ ID NO 1.

In one embodiment the growth hormone compound comprises at least one pair of mutations corresponding to D26N102C, D26/Y103C, S57C/Y143C, 158C/S144C, P59C/Q137C, S71C/S132C, Q84C/Y143C, S85C/Y143C, S85C/S144C, F92C/T148C and/or R94C/D107C in SEQ ID NO 1.

In one embodiment the growth hormone compound comprises at least one pair of mutations corresponding to S57C/Y143C, Q84C/Y143C, S85C/Y143C and/or S85C/S144C in SEQ ID NO 1.

In further embodiments the invention relates to growth hormone conjugates of formula I, wherein the growth hormone compound comprise one or more additional disulfide bond(s) and at least one additional single point mutation compared to human growth hormone. The at least one additional single point mutation may be included in the growth hormone compound for various reasons associated with the functionality of the growth hormone compound. Said at least one additional single point mutation may be aimed at further increasing protease stability and/or providing a “site” suitable for chemical modification, e.g. by introduction of amino acid residues comprising chemical entities suitable for chemical modification.

The invention thus relates to any growth hormone conjugate wherein the growth hormone compound comprises one or more additional disulfide bond(s), as described herein above, and at least one additional single point mutation compared to human growth hormone as defined by SEQ ID NO 1.

In one embodiment the at least one additional single point mutation of the growth hormone compound is at a know protease site.

In one embodiment the at least one additional single point mutation is at a position corresponding to position 1-55, 57, 58, 60-63, 67-87, 89-91, 93, 95-100, 101 102-128, 131-132, 135-139, 141, 142, 144, 148-182, 184, 185 and/or 187-191 of SEQ ID NO 1.

In one embodiment the at least one additional single point mutation is at a position corresponding to position T3, P5, S7, D11, H18, Q29, E30, E33, A34, Y35, E88, Q91, S95, A98, N99, S100, L101, V102, Y103, D107, S108, D112, Q122 and G126 of hGH.

In one embodiment the at least one additional single point mutation is at a position corresponding to position 10, 40, 41, 42, 55, 57, 62, 101, 134, 136, 139, 142 and/or 144 of SEQ ID NO 1.

In one embodiment the at least one additional single point mutation is in a position corresponding to position 55, 57, 62, 101, 134, 136, 142 and/or 144 of SEQ ID NO 1.

In one embodiment the at least one additional single point mutation is at a position corresponding to position 62, 101, 134, 136, 142 and/or 144 of SEQ ID NO 1.

In one embodiment at least one single point mutation is present in L1 (corresponding to AA 36-71 of SEQ ID NO 1) and/or L3 (corresponding to AA 128-154 of SEQ ID NO 1) and in a further embodiment at least one single point mutation is present in L1. In one embodiment at least one single point mutation is present in the middle region of L1 (corresponding to AA 40-65 of SEQ ID NO 1). In one embodiment at least one single point mutation is present in a position corresponding to position 40, 41, 42, 55, 57 and/or 62 of SEQ ID NO 1 In one embodiment at least one single point mutation is present in L3 and in a further embodiment at least one single point mutation is present in the middle region of L3 (corresponding to AA 134-148 of SEQ ID NO 1).

In one embodiment the at least one single point mutation is in a position corresponding to position 134, 136, 139, 142 and/or 144 of SEQ ID NO 1.

In one embodiment the at least one single point mutation is a position corresponding to position 62 of hGH as defined in SEQ ID NO 1.

In one embodiment at least one additional single point mutation is a position corresponding to position 62 of hGH as defined in SEQ ID NO 1. In one such embodiment the Serine (S62) is substituted with an amino acid residue selected from the group of threonine (T), asparagine (N), cysteine (C), histidine (H), glutamine (Q) and glutamic acid (E).

In one embodiment at least one additional single point mutation is a position corresponding to position 55 of hGH as defined in SEQ ID NO 1. In one such embodiment the Serine (S55) is substituted with an amino acid residue selected from the group of threonine (T), asparagine (N), cysteine (C), histidine (H), glutamine (Q) and glutamic acid (E).

In one embodiment at least one additional single point mutation is a position corresponding to position 57 of hGH as defined in SEQ ID NO 1. In one such embodiment the Serine (S57) is substituted with an amino acid residue selected from the group of threonine (T), asparagine (N), cysteine (C), histidine (H), glutamine (Q) and glutamic acid (E).

According to the invention the one or more additional disulfide bond(s) is/are obtained by amino acid substitution of at least two amino acids compared SEQ ID NO 1.

In a further embodiment the compound comprises exactly one additional disulfide bond compared to SEQ ID NO 1.

In one embodiment the polypeptide of a growth hormone compound according to the invention comprises at least two additional cysteines compared to human growth hormone as defined in SEQ ID NO 1.

In a further embodiment the polypeptide comprises exactly two additional cysteines compared to human growth hormone as defined in SEQ ID NO 1.

In a further embodiment the growth hormone compound comprises at least two additional cysteines compared to human growth hormone as defined in SEQ ID NO 1.

In one embodiment the growth hormone compound comprises exactly 3 amino acid substitutions compared to SEQ ID NO 1.

In one embodiment the growth hormone compound comprises exactly two additional cysteines and exactly one additional single point mutation compared to human growth hormone as defined in SEQ ID NO 1.

In one embodiment the polypeptide of a growth hormone compound according to the invention comprises a total of at most 10 amino acid substitutions compared to SEQ ID N0 1. In one embodiment the growth hormone compound comprises a total of at most 8, such as at most 7, such as at most 6, such as at most 5, such as at most 4 amino acid substitutions. In one embodiment the growth hormone compound comprises exactly three additional cysteines compared to human growth hormone as defined in SEQ ID NO 1.

In one embodiment the growth hormone compound comprises one additional disulphide bond and a single cys mutation compared to human growth hormone as defined in SEQ ID NO 1.

In one embodiment the single Cys mutation is selected from any one of a single Cys mutation in the N-terminal, H1, H2, L2 or H3 regions of GH.

In one embodiment the growth hormone compound has a single Cys mutation selected from the group of mutations corresponding to: T3C, P5C, S7C, D11C, H18C, Q29C, E300, E33C, A340, Y35C, Q400, S55C, S57C, S62C, E88C, Q91C, S95C, A98C, N99C, S100C, L101C, V102C, Y103C, D107C, S108C, D112C, Q122C and G126C in hGH (SEQ ID NO:1).

In one embodiment the growth hormone compound has a single Cys mutation located in a position corresponding to AA 93-106, such as AA 99-106 or AA 99-103 in hGH.

In one embodiment the additional single cys mutation is N99C, S1000 or L101C. In one embodiment the additional single cys mutation is located in a position corresponding to AA 55-62 in hGH. In one embodiment the additional single cys mutation is S55C, S57C or S62C.

In one embodiment the polypeptide of a growth hormone compound according to the invention comprises a total of at most 10 amino acid substitutions compared to SEQ ID NO 1. In one embodiment the growth hormone compound comprises a total of at most 8, such as at most 7, such as at most 6, such as at most 5, such as at most 4 amino acid substitutions.

In one embodiment the polypeptide of the growth hormone compound according to the invention comprises exactly three amino acid substitutions compared to SEQ ID N0 1.

Other examples of GH compounds into which additional disulphide bridges may be introduced include those disclosed in WO 92/09690 (Genentech), U.S. Pat. No. 6,004,931 (Genentech), U.S. Pat. No. 6,143,523 (Genentech), U.S. Pat. No. 6,136,536 (Genentech), U.S. Pat. No. 6,057,292 (Genentech), U.S. Pat. No. 5,849,535 (Genentech), WO 97/11178 (Genentech), WO 90/04788 (Genentech), WO 02/055532 (Maxygen APS and Maxygen Holdings), U.S. Pat. No. 5,951,972 (American Cynanamid Corporation), US 2003/0162949 (Bolder Biotechnologies, Inc.) which are incorporated herein by reference. Further included are natural variants of hGH, such as the 20 kDa described by Masuda, N et al, Biochim. Biophys. Acta 949 (1988) (1), 125-131.

In all embodiments described herein it is a further option that the growth hormone compound has a Gly residue in a position corresponding to position 120 of SEQ ID NO 1.

The growth hormone polypeptides described herein can be prepared according to Method 1 and 2 and as described herein below by the person skilled in the art.

The production of polypeptides is well known in the art. For example, polypeptides may be produced by classical peptide synthesis, e.g. solid phase peptide synthesis using tert-Boc or Fmoc chemistry or other well established techniques, see e.g. Greene and Wuts, “Protective Groups in Organic Synthesis”, John Wiley & Sons, 2006.

The polypeptides may also be produced by a method which comprises culturing a host cell containing a DNA sequence encoding the polypeptide and capable of expressing the polypeptide in a suitable nutrient medium under conditions permitting the expression of the peptide. For polypeptides comprising non-natural amino acid residues, the recombinant cell should be modified such that the non-natural amino acids are incorporated into the polypeptide, for instance by use of tRNA mutants.

The polypeptides may also be produced using cell-free in vitro transcription/-translation systems. A polypeptide containing novel unnatural amino acids may also be produced using frame shift or nonsense suppression systems e.g. as described in J. Am. Chem. Soc. (2003) 125, 11782-11783, Science (2003) 301, 964-967, Science (2001) 292, 498-500, Science (2004) 303, 371-373 and references herein.

The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The peptide produced by the cells may then be recovered from the culture medium by conventional procedures including separating the host cells from the medium by centrifugation or filtration. For extra cellular products the proteinaceous components of the supernatant are isolated by filtration, column chromatography or precipitation, e.g. microfiltration, ultrafiltration, isoelectric precipitation, purification by a variety of chromatographic procedures, e.g. ion exchange chromatography, hydrophobic interaction chromatography, gel filtration chromatography, affinity chromatography, or the like, dependent on the type of polypeptide in question. For intracellular or periplasmic products the cells isolated from the culture medium are disintegrated or permeabilized and extracted to recover the product polypeptide or precursor thereof.

The DNA sequence encoding the polypeptide may suitably be of genomic or cDNA origin, for instance obtained by preparing a genomic or cDNA library and screening for DNA sequences coding for all or part of the peptide by hybridisation using specific DNA or RNA probes in accordance with standard techniques (see, for example, Sambrook, J, Fritsch, E F and Maniatis, T, Molecular Cloning: A Laboratory Manual, Cold Spring Harbor Laboratory Press, New York, 1989). The DNA sequence encoding the polypeptide may also be prepared synthetically by established standard methods, e.g. the phosphoamidite method described by Beaucage and Caruthers, Tetrahedron Letters (1981) 22, 1859-1869, or the method described by Matthes et al., EMBO Journal (1984) 3, 801-805. The DNA sequence may also be prepared by polymerase chain reaction using specific primers, for instance as described in U.S. Pat. No. 4,683,202 or Saiki et al., Science (1988) 239, 487-491.

The DNA sequence encoding the peptide to be expressed may be inserted into any vector which may conveniently be subjected to recombinant DNA procedures, and the choice of vector will often depend on the host cell into which it is to be introduced. Thus, the vector may be an autonomously replicating vector, i.e. a vector which exists as an extra-chromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector may be an expression vector in which the DNA sequence encoding the polypeptide is operable linked to additional segments required for transcription of the DNA, such as a promoter. The promoter may be any DNA sequence which shows transcriptional activity in the host cell of choice and may be derived from genes encoding proteins either homologous or heterologous to the host cell. Examples of suitable promoters for directing the transcription of the DNA encoding the peptide to be expressed in a variety of host cells are well known in the art, cf. for instance Sambrook et al., supra.

The DNA sequence encoding the peptide to be expressed may also, if necessary, be operably connected to a suitable terminator, polyadenylation signals, transcriptional enhancer sequences, and translational enhancer sequences. The recombinant vector of the invention may further comprise a DNA sequence enabling the vector to replicate in the host cell in question.

The vector may also comprise a selectable marker, for instance a gene the product of which complements a defect in the host cell or one which confers resistance to a drug, for instance ampicillin, kanamycin, tetracyclin, chloramphenicol, neomycin, hygromycin or methotrexate. For large scale manufacture the selectable marker may for instance not be antibiotic resistance, e.g. antibiotic resistance genes in the vector may be excised when the vector is used for large scale manufacture. Methods for eliminating antibiotic resistance genes from vectors are known in the art, see e.g. U.S. Pat. No. 6,358,705 which is incorporated herein by reference.

To direct the peptide to be expressed into the secretory pathway of the host cells, a secretory signal sequence (also known as a leader sequence, prepro-sequence or presequence) may be provided in the recombinant vector. The secretory signal sequence is joined to the DNA sequence encoding the peptide in the correct reading frame. Secretory signal sequences are commonly positioned 5′ to the DNA sequence encoding the peptide. The secretory signal sequence may be that normally associated with the peptide or may be from a gene encoding another secreted protein.

The procedures used to ligate the DNA sequences coding for the peptide to be expressed, the promoter and optionally the terminator and/or secretory signal sequence, respectively, and to insert them into suitable vectors containing the information necessary for replication, are well known to persons skilled in the art (cf., for instance, Sambrook et al., supra).

The host cell into which a DNA sequence or recombinant vector is introduced may be any cell which is capable of producing the present peptide and includes bacteria, yeast, fungi and higher eukaryotic cells. Examples of suitable host cells well known and used in the art are, without limitation, E. coli, Saccharomyces cerevisiae, or mammalian BHK or CHO cell lines. The peptide to be expressed can also be produced by using in vitro transcription/translation systems commonly known in the art.

### Bile Acid Linker

The present invention in an aspect relates to a bile acid linker. A growth hormone conjugate according to the invention may be prepared as described in the Chemistry section below. The method of conjugation involves covalent linkages of a bile acid linker, which dependent on the method employed may be described by the formulas:

A-W—B1-NH₂,A-W—B1-CHO,A-W—B1-LG,A-W—B1-C(O)NHCH₂—CH═CH₂ and

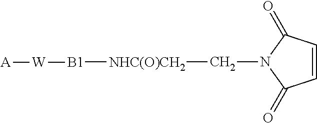

wherein A represents a bile acid residue, B1 represents a hydrophilic spacer, and W is a chemical group linking A and B1. Further details with regards to A, W and B1 is provide below in relation to the bile acid residue, the spacer and the linking group W.

B1 may be seen as a part of B as described in the following:

A-W—B1-NH₂->A-W—B1-NH-GH->A-W—B-GH(B═B1-NH)

A-W—B1-CHO->A-W—B1-CH₂-GH->A-W—B-GH(B═B1-CH₂—)

A-W—B1-LG->A-W—B1-GH->A-W—B-GH(B1=B)

A-W—B1-C(O)NHCH₂—CH═CH₂->A-W—B1-C(O)NHCH₂—CH₂—CH₂-GH->A-W—B-GH

(B═B1-C(O)NHCH₂—CH₂—CH₂—) and

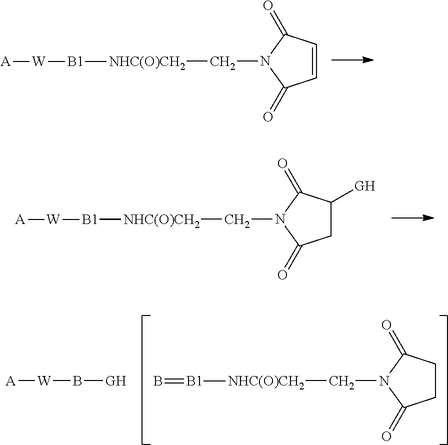

### Bile Acid Residue

Bile acids occur in nature in various forms which may additionally be subject to modification prior to conjugation to the growth hormone compound.

According to the invention the growth hormone conjugate comprise a bile acid residue (A) selected from the group consisting of: cholanic acid, cholic acid, chenodeoxycholic acid, deoxycholic acid, ursodeoxycholic acid, lithocholic acid, glycocholate, glycodeoxycholate, glycochenodeoxycholate, taurocholate, taurodeoxycholate and taurochenodeoxycholate.

In one embodiment the bile acid linker and/or the growth hormone conjugate comprises a bile acid residue (A) selected from the group consisting of:

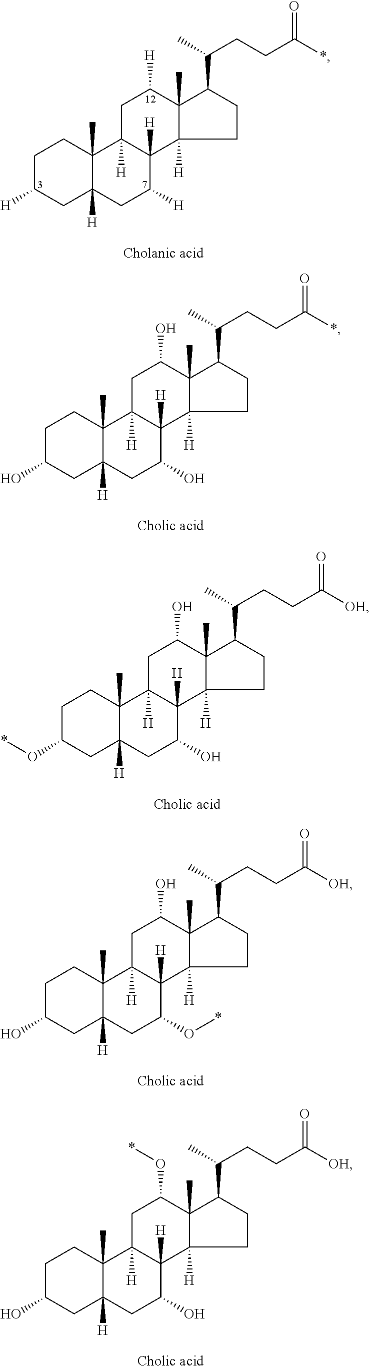

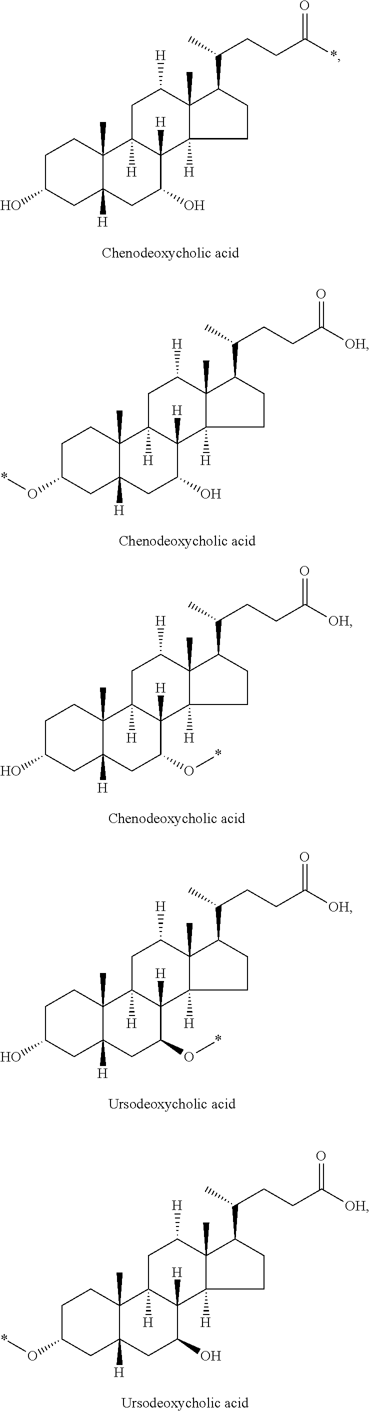

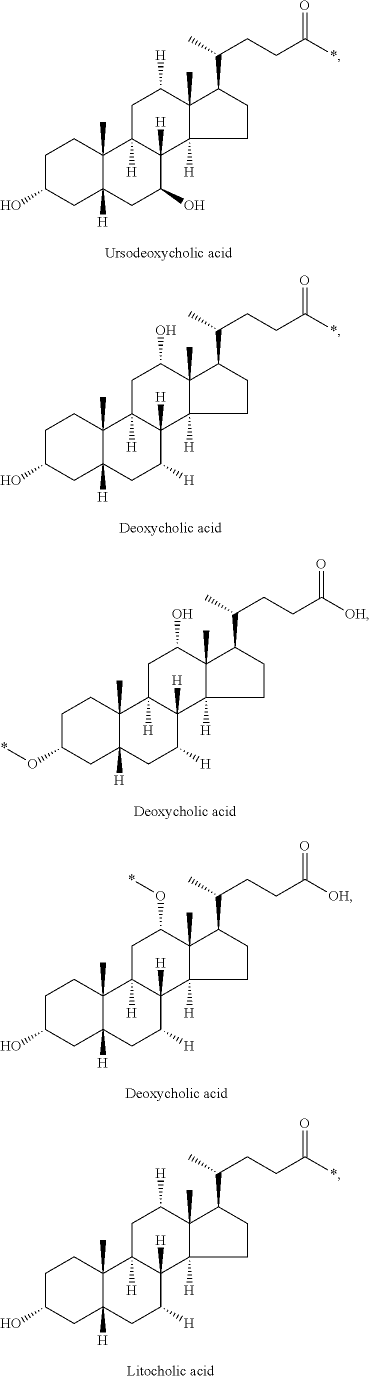

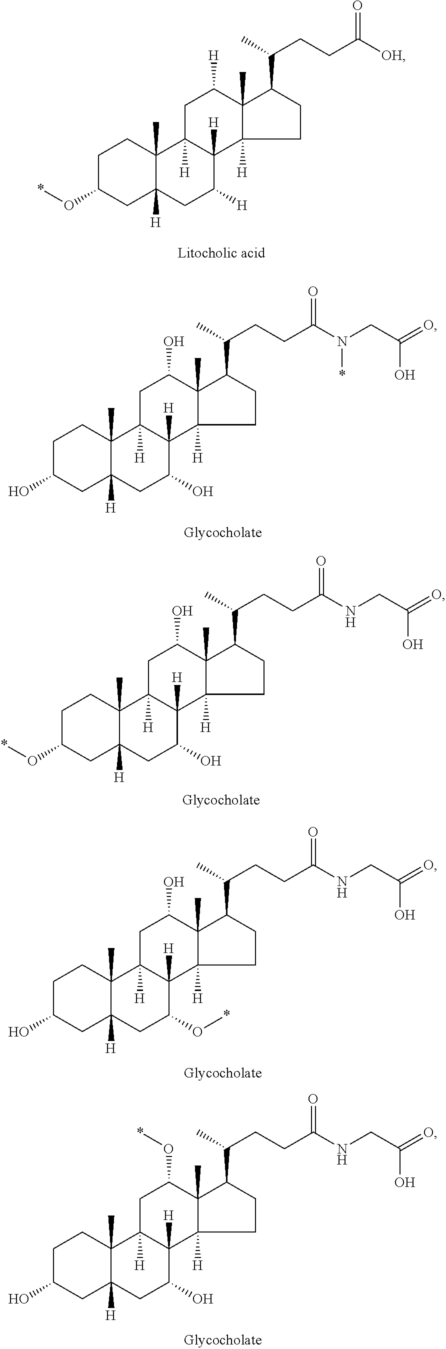

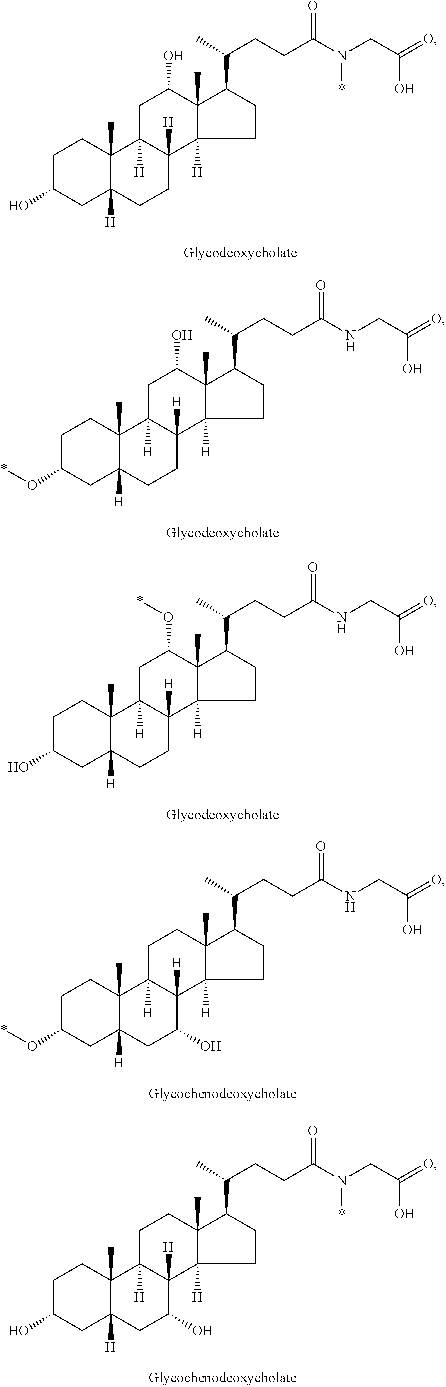

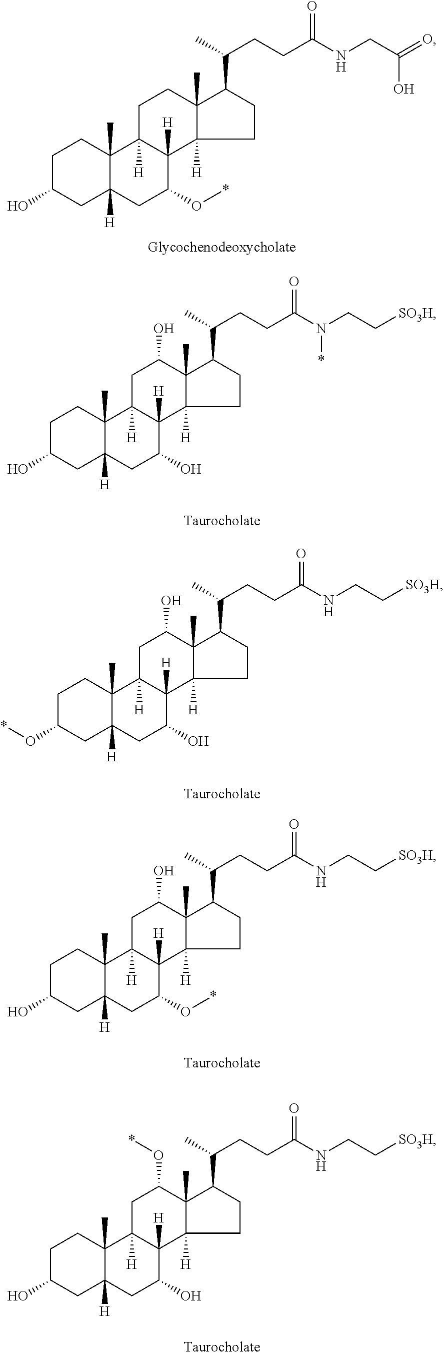

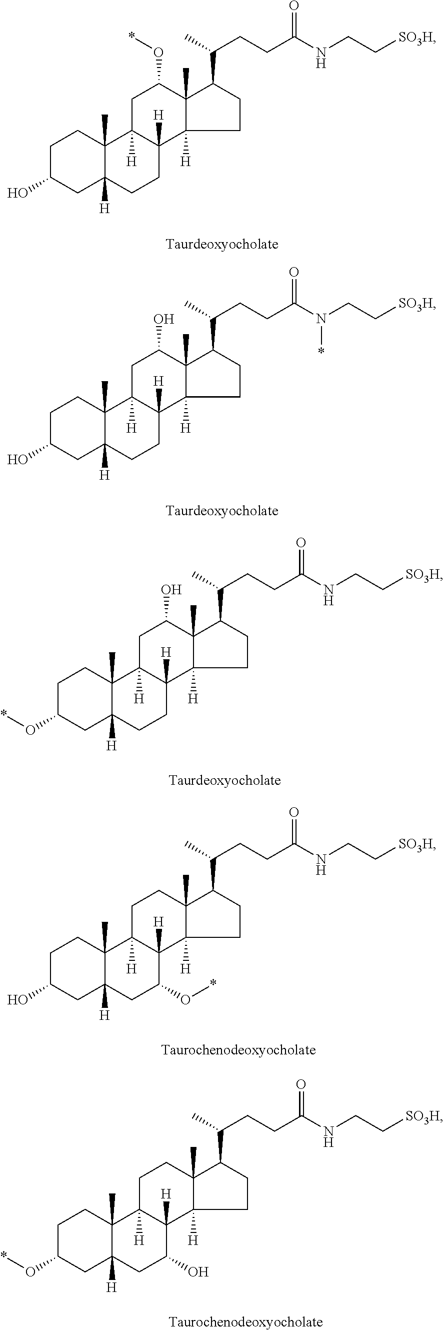

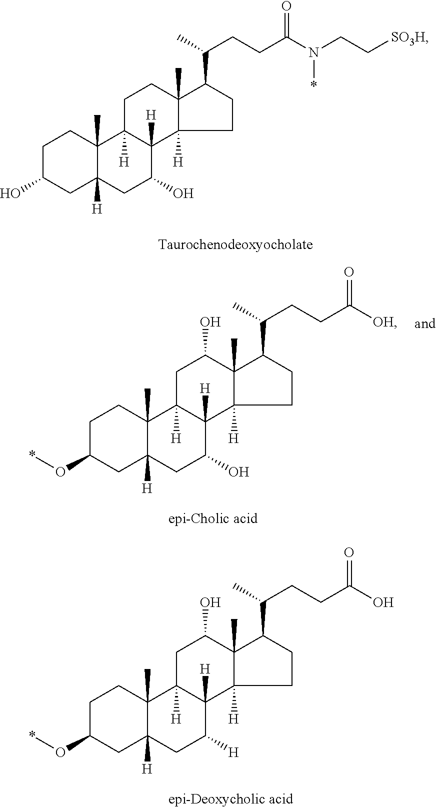

wherein (*) represents the attachment point to the hydrophilic spacer (B) through W.

In one embodiment the bile acid residue (A) is a cholic acid residue, such a cholic acid residue is selected from:

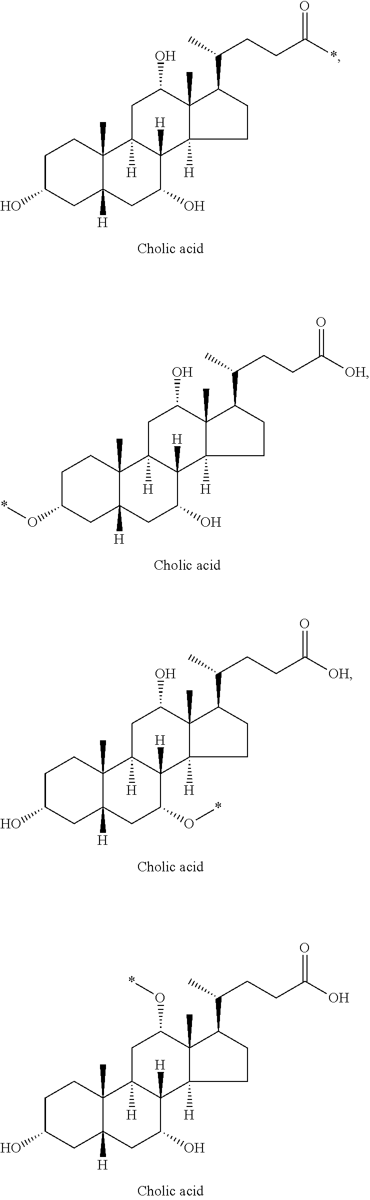

and wherein (*) represents the attachment point to the hydrophilic spacer (B) through W.

As seen from the above the cholic acid residue may in one embodiment be linked to the hydrophilic spacer (B) through W via position “3”, “7”, “12” or “24” of the cholic acid residue.

In one embodiment the cholic acid residue is linked to the hydrophilic spacer (B) through W via position “3”, “7” or “12”.

In one embodiment the cholic acid residue is linked to the hydrophilic spacer (B) through W via position “3”.

In one embodiment the cholic acid residue is linked to the hydrophilic spacer (B) through W via position “7”.

In one embodiment the cholic acid residue is linked to the hydrophilic spacer (B) through W via position “12”.

### Hydrophilic Spacer

The bile acid linker and/or growth hormone conjugate is further characterized by the presence of a hydrophilic spacer. The hydrophilic spacer is aimed and providing a suitable distance from the growth hormone compound and the bile acid residue in order to optimize the functionality of the bile acid linker. Additional functionalities may be associated with groups of spacers as well as individual linkers, such as superior synthesis of conjugates or superior secondary effects.

Solubility of a hydrophilic spacer can be described by its logP value. LogP, also known as the partition coefficient, is the logarithm of the ratio of concentrations of a compound in the two phases of a mixture of two immiscible solvents at equilibrium. Typically one of the solvents is water while the second is selected from octan-1-ol, chloroform, cyclohexane and propylene glycol dipelargonate (PGDP). LogP values measured in these different solvents show differences principally due to hydrogen bonding effects. Octanol can donate and accept hydrogen bonds whereas cyclohexane is inert. Chloroform can donate hydrogen bonds whereas PGDP can only accept them.

In one embodiment of the invention, the hydrophilic spacer has a LogP below −0.5 in either octan-1-ol, chloroform, cyclohexane and propylene glycol dipelargonate (PGDP).

In a further embodiment, the hydrophilic spacer has a logP below −1 in either octan-1-ol, chloroform, cyclohexane and propylene glycol dipelargonate (PGDP).

Alternatively, the LogP value can be calculated as mLogP and/or cLogP for the hydrophilic spacer part using published algorithms (J. Am. Chem. Soc (1964), 86 5175-5180 “A New Substituent Constant, Derived from Partition Coefficients”, C. A. Lipinski et al. Advanced Drug Delivery Reviews, (1997) 23 3-25, “Experimental and Computational Approaches to Estimate Solubility and Permeability in Drug Discovery and Development Settings” and I. Moriguchi, S. Hirono, I. Nakagome, H. Hirano, Chem. and Pharm. Bull., (1994) 42 976-978 “Comparison of Reliability of logP Values for Drugs Calculated by Several Methods”.

In one embodiment of the present invention the hydrophilic spacer has a mLogP<0. In a further embodiment, the mLogP of the hydrophilic space is below −0.50, such as below −1.00. such as below −1.50, −2.00, −2.50, −3.00, −3.50, −4.00 or 4.50.

In one embodiment of the present invention the hydrophilic spacer has a cLogP<0. In a further embodiment, the cLogP of the hydrophilic space is below −0.50, such as below −1.00. such as below −1.50, −2.00, −2.50, −3.00, −3.50, −4.00 or 4.50 or such as below −5.00, −5.50 or −6.00.

When conjugating property modifying groups, such as a cholic acid residue, to polypeptides and proteins the distance between the protein and the cholic acid residue may influence the functionality of the final compound, and thus the use of a hydrophilic spacer may be advantageous. The use of a hydrophilic spacer may increase aqueous solubility of the final compound as well as increase yield and ease of synthesis of such compounds as the conjugation reaction between a linker including a hydrophilic spacer and the protein, usually is performed in a buffered aqueous environment. As seen here below, a hydrophilic spacer as used in the present invention provides a number of negative charges in the form of COOH groups and/or additional polarity in the from of (—CH2-CH2-O—)n repeats, which may both individally influence the solubility positively.

In one embodiment the hydrophilic spacer is a spacer that separates a growth hormone compound and the bile acid residue with a chemical moiety which comprises at least 5 nonhydrogen atoms where 30-50% of these are either N or O.

In one embodiment the hydrophilic spacer is a spacer that separates a growth hormone compound and the bile acid residue with a chemical moiety which comprises from 0 to 5 COOH groups.

In one embodiment the hydrophilic spacer is a spacer that separates a growth hormone compound and the bile acid residue with a chemical moiety which comprises from 0 to 100-CH₂—CH₂—O— groups.

In one embodiment the hydrophilic spacer (B) comprises at least one OEG motif, the radical 8-amino-3,6-dioxaoctanic acid, i.e. —NH—(CH₂)₂—O—(CH₂)₂—O—CH₂—CO—. In further embodiments the hydrophilic spacer comprises at least two OEG motifs. The orientation of such OEG motif(s) is in one embodiment so that the —CO— is closest to the growth hormone compound and the —NH— is closest to the albumin binding residue.

In additional embodiments comprising two OEG motifs the two motifs have identical orientation or different orientation. In an embodiment two such OEG motifs are located adjactant to each other whereas in alternative embodiments such OEG motifs are separated by one or more atoms covalently linked.

In an embodiment the hydrophilic spacer comprise at lease one glutamic acid residue. The glutamic acid comprises two carboxylic acid groups. Its gamma-carboxy group may be used for forming an amide bond with the epsilon-amino group of lysine, or with an amino group of an OEG molecule, if present, or with the amino group of another Glu residue, if present. The alfa-carboxy group may alternatively be used for forming similar amide bond with the epsilon-amino group of lysine, or with an amino group of an OEG molecule, if present, or with the amino group of another Glu residue, if present. The amino group of Glu may in turn form an amide bond with the carboxy group of the albumin binding residue, or with the carboxy group of an OEG motif, if present, or with the gamma-carboxy group or alfa carboxy group of another Glu, if present.

In one embodiment the hydrophilic spacer (B) may comprises two or more glutamic acid residue. The linkage of the amino group of one Glu to a gamma-carboxy group of a second Glu may be referred to as a “gamma-Glu” motif.

In one embodiment the hydrophilic spacer comprise at lease one combined OEG-Glu motif (—NH—(CH₂)₂—O—(CH₂)₂—O—CH₂—CO—NH—CH(—COOH)—(CH₂)₂—CO—) or at least one combined Glu-OEG motif (—NH—CH(—COOH)—(CH₂)₂—CO—NH—(CH₂)₂—O—(CH₂)₂—O—CH₂—CO—) or combination here of, where in such Glu-OEG and OEG-Glu motifs may be separated by one or more covalently liked atoms or directly bond to each other by an amide bond of the Glu's forming a gammal-Glu.

In one embodiment the hydrophilic spacer (B) has the formula

—X₁—X₂—X₃—X₄—

wherein
X₁ is —W₁—[(CHR³)ₗ₁—W₂]ₘ₁-{[(CH₂)ₙ₁E1]ₘ₂—[(CHR⁴)ₗ₂—W₃]ₘ₃}ₙ₂-,
X₂ is —[(CHR⁵)ₗ₃—W₄]ₘ₄-{[(CH₂)ₙ₃E2]ₘ₅—[(CHR⁶)ₗ₄—W₅]ₘ₆}ₙ₄-,
X₃ is —[(CHR⁷)ₗ₅—W₆]ₘ₇-,
X₄ is F-D1-(CH₂)₁₆-D2-,
l1, l2, l3, l4, l5 and l6 independently are selected from 0-10,
m1, m3, m4, m6 and m7 independently are selected from 0-6,
m2 and m5 independently are selected from 0-6,
n1, n2, n3 and n4 independently are selected from 0-6,
F is aryl, hetaryl, pyrrolidine-2,5-dione or a valence bond,
wherein the aryl and hetaryl groups are optionally substituted with halogen, —CN,
—OH, —C(O)OH, —C(O)NH₂, —S(O)₂OH or C₁₋₆-alkyl,
R³, R⁴, R⁵, R⁶ and R⁷ independently are selected from hydrogen, —C(O)OH, —C(O)NH₂, —S(O)OH, —S(O)₂OH, —NH—C(═NH)—NH₂, C₁₋₆-alkyl, aryl or hetaryl;
wherein the alkyl, aryl and hetaryl groups optionally are substituted with halogen, —C(O)OH, —C(O)OH, —C(O)NH₂, —S(O)OH, —S(O)₂OH, —CN or —OH,

D1, D2, E1 and E2 independently are selected from —O—, —N(R⁸)—, —N(C(O)R⁹)— or a valence bond; wherein R⁸ and R⁹ independently represent hydrogen or C₁₋₆-alkyl, W₁ to W₆ independently are selected from —NH—, —C(O)NH—, —NHC(O)—, —C(O)NHCH₂—, —CH₂NHC(O)—, —C(O)NHS(O)₂—, —S(O)₂NHC(O)—, —OC(O)NH—, —NHC(O)O—, —C(O)CH₂—, —NHC(O)C₁₋₆-alkyl, —C(O)NHC₁₋₆-alkyl, —CH₂C(O)—, —C(O)CH═CH—, —CH═CHC(O)—, —(CH₂)ₛ₂—, —C(O)—, or a valence bond; wherein s2 is 0 or 1.

In further specified embodiments the space is as defined above, wherein

l1, l2, l3, l4, l5 and l6 independently are 0-6,
m1, m3, m4, m6 and m7 independently are 0-6,
m2 and m5 independently are 0-6, and
n1, n2, n3 and n4 independently are 0-6.

In further specified embodiments the space is as defined above, wherein D1 and D2 are independently selected from —O— or a valence bond.

In further specified embodiments the space is as defined above, wherein E1 and E2 are independently selected from —O— or a valence bond.

In further specified embodiments the space is as defined above, wherein W₁ through W₆ independently are selected from the group consisting of: —NH—, —C(O)NH—, —NHC(O)—, —CH₂NHC(O)—, —C(O)NHS(O)₂—, —S(O)₂NHC(O)—, —NHC(O)C₁₋₆-alkyl or —C(O)NHC₁₋₆-alkyl and a valence bond.

In further specified embodiments the space is as defined above, wherein R³, R⁴, R⁵, R⁶ and R⁷ independently are selected from hydrogen, —C(O)OH, —C(O)NH₂, —S(O)₂OH or C₁₋₆-alkyl; wherein the alkyl group optionally is substituted with —C(O)OH, —C(O)NH₂ or —S(O)₂OH.

In an additional embodiment of the invention the growth hormone conjugate comprises a hydrophilic spacer (B) selected from:

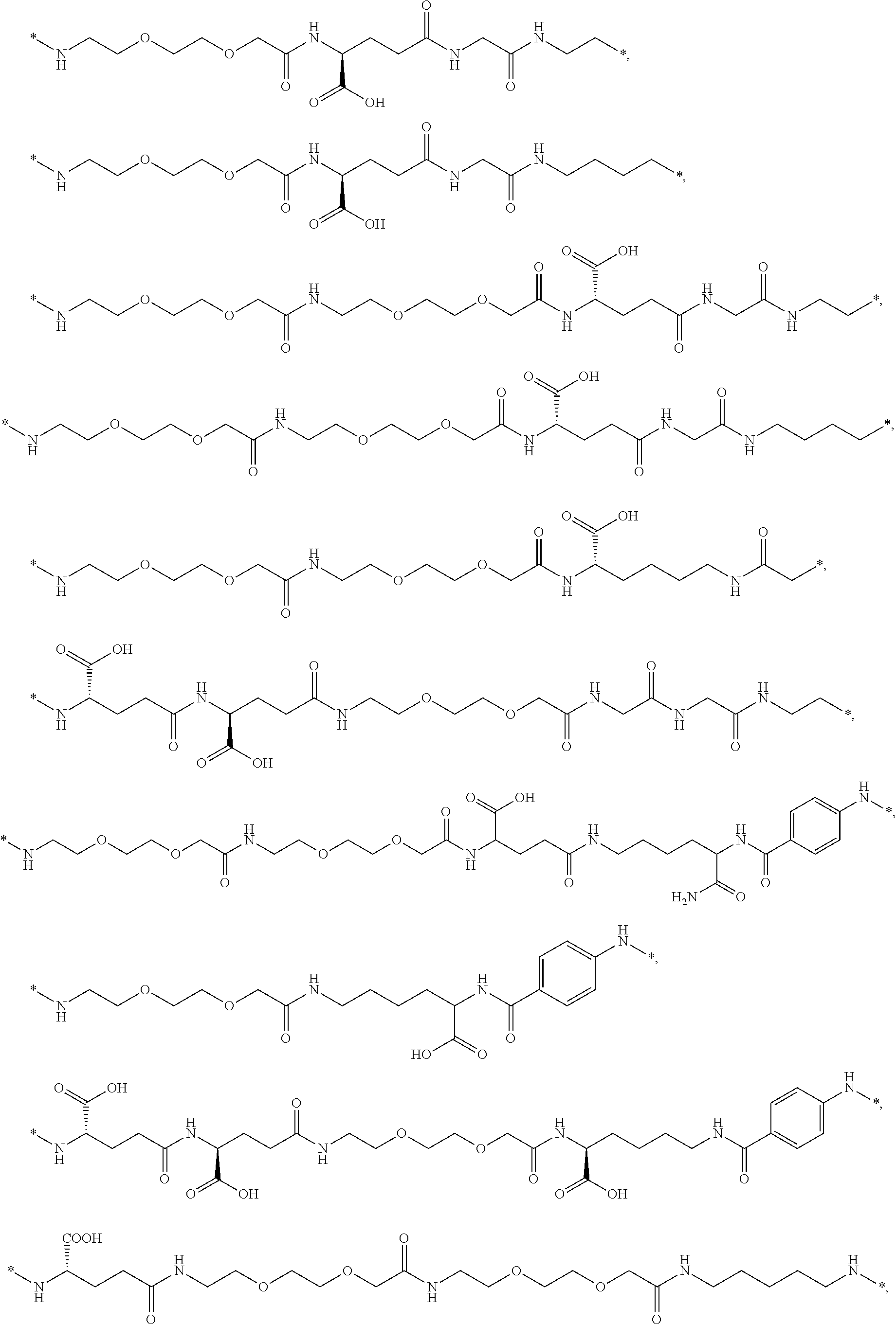

### Chemical Linking Group

The bile acid linker and/or growth hormone conjugate is further characterized by the chemical linking group (w) which is determined by the method of synthesis of the cholic acid linker. The chemical linking group is a result of linkage of the bile acid residue (A) and the hydrophilic spacer (B) or rather the intermediates used to synthesis the linker as chemical active groups may be altered during synthesis of the bile acid linker (A-W—B1-X), wherein X is a leaving group (LG) e.g. a halogen or a chemically reactive group which can participate in forming a covalent chemical bond between B1 and the compound being conjugated, e.g. hGH or an alternative compound.

In one embodiment the chemical group (W) is selected from: —NR¹—, —NHC(O)[CH₂]ₙ₁C(O)—, —NHC(O)CH═CHC(O)—, —C(O)NH—, —NHC(O)—, —C(O)NHCH₂—, —CH₂NHC(O)—, —C(O)NHS(O)₂—, —S(O)₂NHC(O)—, —NHC(O)O—, —C(O)CH₂—, —CH₂C(O)—, —C(O)CH═CH—, —CH═CHC(O)—, or —C(O)—;

wherein R¹ is selected from hydrogen or —[CH₂]ₙ₂R², n2 is 1 to 4 and R² is selected from —C(O)OH, —S(O)₂OH or tetrazol-1-yl and wherein n1 is 2 to 8.
In one embodiment the chemical group (X) is selected from: —NH₂, —CHO, -LG, —C(O)NHCH₂—CH═CH₂, or

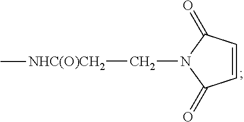

wherein LG is a halogen.

### Growth Hormone Conjugates

The growth hormone conjugates are as described here formed by covalent linking of a growth hormone compound and bile acid linker as described herein.

The bile acid linker may be covalently bound to any residue of the growth hormone compound as long as the activity of the GH is maintained as described in a prior section. In one embodiment GH is conjugated at a wild type amino acid residue.

In one embodiment GH is conjugated at a wild type amino acid residue selected from the group of: the N-terminal, the C-terminal, Gln40 and Gln141.

In one embodiment GH is conjugated at a mutant residue.

In one embodiment GH is conjugated at a mutant residue, which is a cys residue introduced in the growth hormone compound as a single point mutation.

In one embodiment the growth hormone conjugate is selected from the group consisting of:

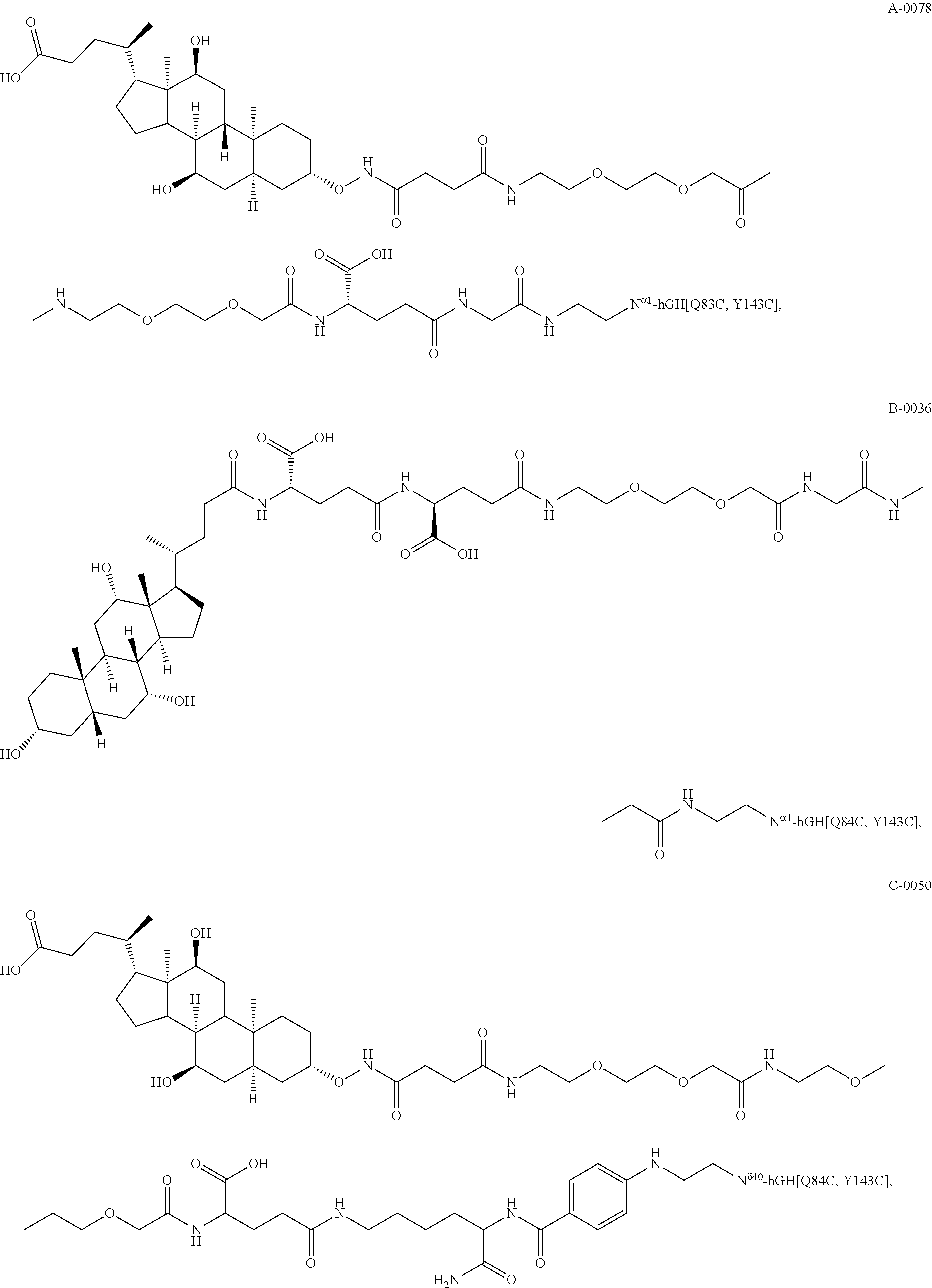

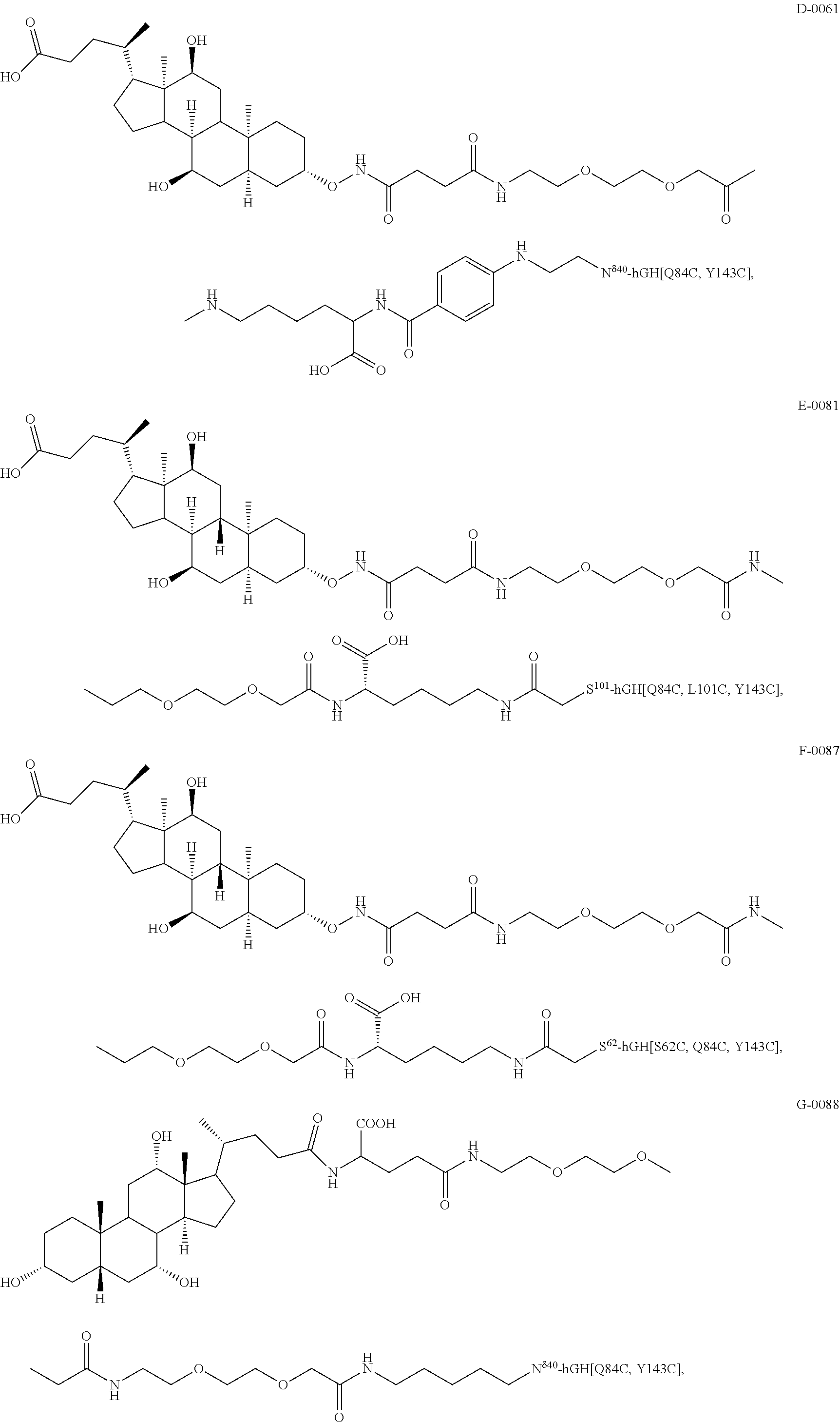

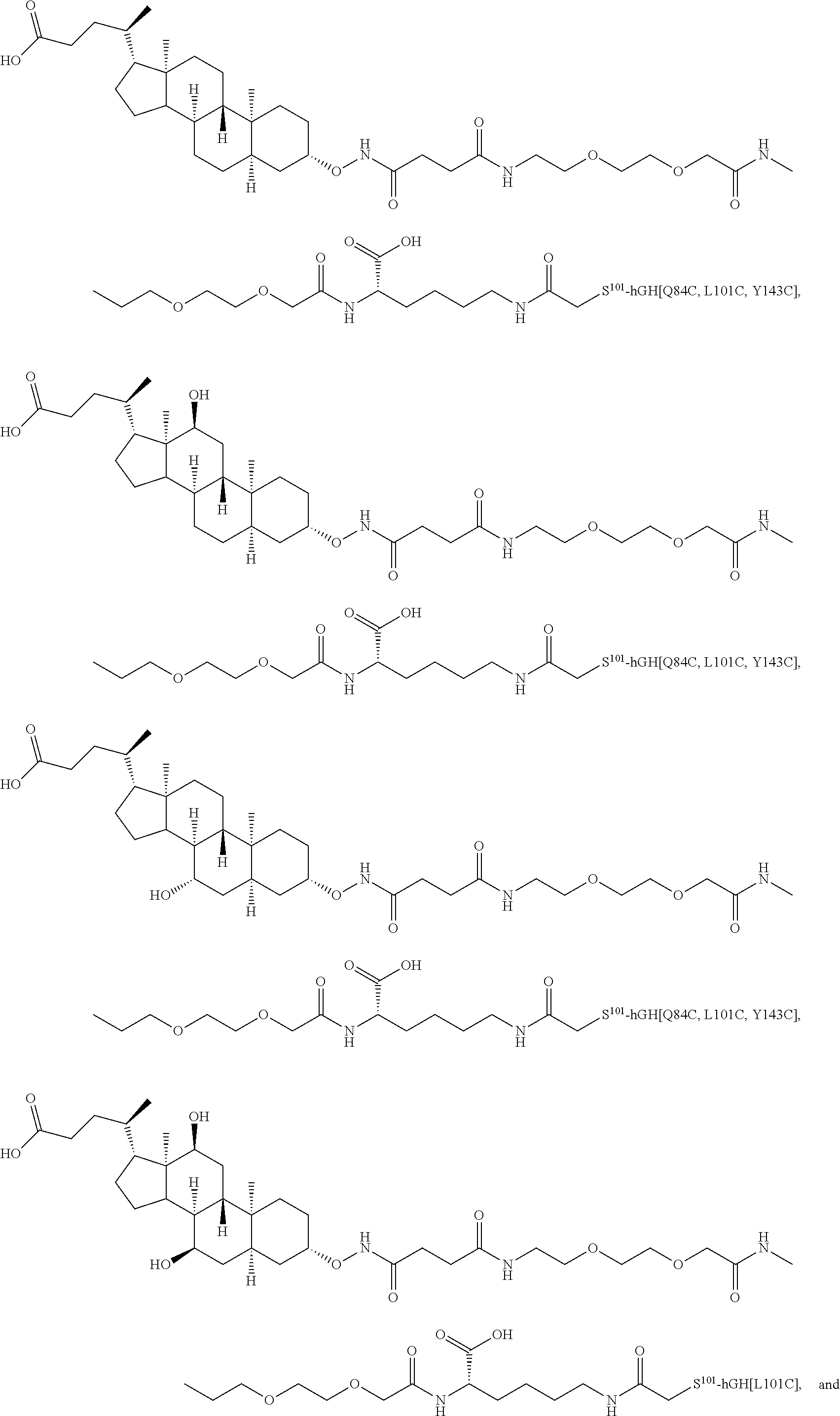

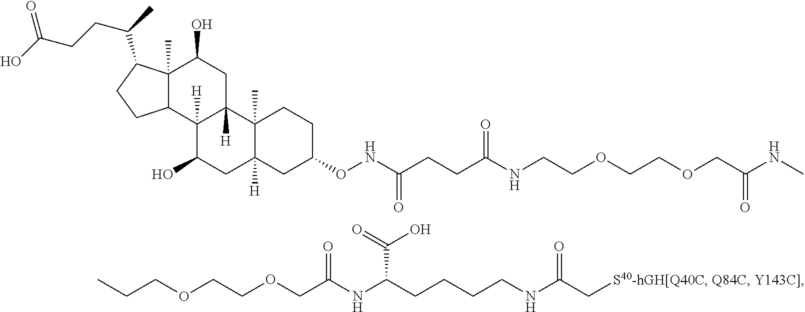

### Method of Preparing Growth Hormone Conjugates

The chemistry section provided herein below describes methods suitable for preparing growth hormone conjugates according to the invention. A person skilled in the art may see alternative method of preparation of such conjugates.

In one aspect the invention relates to a method for preparing a growth hormone conjugate, which method comprises the step of:

a) conjugating a bile acid linker to a growth hormone compound (GH) and
b) obtaining a growth hormone conjugate.

In one embodiment the growth hormone conjugate obtained is defined by formula I as described herein, wherein A represents a bile acid residue, B represents a hydrophilic spacer, and W is a chemical group linking A and B.

In one embodiment the bile acid linker employed in the method is selected from the group of:

A-W—B1-NH₂,A-W—B1-CHO,A-W—B1-LG,A-W—B1-C(O)NHCH₂—CH═CH₂ and

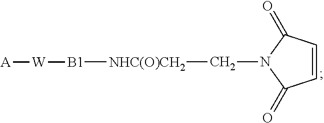

wherein A represents a bile acid residue, B1 represents a hydrophilic spacer, and W is a chemical group linking A and B1.

Base on the description herein it is clear that B1 is related to B as defined in relation to the hydrophilic space of the growth hormone conjugate obtained.

In one embodiment the hydrophilic spacer B1 comprises any of the features of the hydrophilic spacer B. In one embodiment the hydrophilic spacer B1 is comprised by the hydrophilic spacer B of the growth hormone conjugate obtained.

In one embodiment the hydrophilic spacer hydrophilic spacer B1 has the formula: —X₁—X₂—X₃—X₄—

wherein
X₁ is —W₁—[(CHR³)ₗ₁—W₂]ₘ₁-{[(CH₂)ₙ₁E1]ₘ₂—[(CHR⁴)ₗ₂—W₃]ₘ₃}ₙ₂-,
X₂ is —[(CHR⁵)ₗ₃—W₄]ₘ₄-{[(CH₂)ₙ₃E2]ₘ₅—[(CHR⁶)ₗ₄—W₅]ₘ₆}ₙ₄-,
X₃ is —[(CHR⁷)ₗ₅]ₘ₇-,
X₄ is a valence bond,
l1, l2, l3, l4, and l5 independently are selected from 0-10,
m1, m3, m4, m6 and m7 independently are selected from 0-6,
m2 and m5 independently are selected from 0-6,
n1, n2, n3 and n4 independently are selected from 0-6,
R³, R⁴, R⁵, R⁶ and R⁷ independently are selected from hydrogen, —C(O)OH,
—C(O)NH₂, —S(O)OH, —S(O)₂OH, —NH—C(═NH)—NH₂, C₁₋₆-alkyl, aryl or hetaryl;
   wherein the alkyl, aryl and hetaryl groups optionally are substituted with
   halogen, —C(O)OH, —C(O)NH₂, —S(O)OH, —S(O)₂OH, —CN or —OH, E1 and E2 independently are selected from —O—, —NR⁸—, —N(COR⁹)— or a valence bond;
   wherein R⁸ and R⁹ independently represent hydrogen or C₁₋₆-alkyl, W₁ to W₆ independently are selected from —NH—, —C(O)NH—, —NHC(O)—, —C(O)NHCH₂—, —CH₂NHC(O)—, —C(O)NHS(O)₂—, —S(O)₂NHC(O)—, —OC(O)NH—, —NHC(O)O—, —C(O)CH₂—, —CH₂C(O)—, —C(O)CH═CH—, —CH═CHC(O)—, —(CH₂)ₛ₂—, —C(O)—, —C(O)O—, —C(O)—, or a valence bond; wherein s2 is 0 or 1.

### Pharmaceutical Composition

The present invention is also directed towards pharmaceutical compositions comprising growth hormone conjugates as defined and described herein.

In one embodiment, pharmaceutical compositions of the present invention may be administered in several dosage forms, for example, as solutions, suspensions, emulsions, microemulsions, multiple emulsion, foams, salves, pastes, plasters, ointments, tablets, coated tablets, tablets with co-formulation of absorption enhancing compounds, rinses, capsules, for example hard gelatine capsules and soft gelatine capsules, coated capsules, suppositories, drops, gels, sprays, powder, microparticles, nanoparticles, aerosols, inhalants, injection solution, in situ transforming solutions, for example in situ gelling, in situ setting, in situ precipitating, in situ crystallization, infusion solution, and implants.

In one embodiment of the present invention, the pharmaceutical compositions may be administered through oral, subcutaneous, intramuscular, nasal and i.v administration.

In one embodiment of the present invention, the oral pharmaceutical compositions may be administered through several routes of administration, for example, lingual, sublingual, buccal, in the mouth, rectal, in the stomach and intestine.

In one embodiment, pharmaceutical compositions of present invention are useful in the composition of solids, semisolids, powder and solutions for pulmonary administration of a peptide conjugate, such as e.g. a GH conjugate, using, for example a metered dose inhaler, dry powder inhaler and a nebulizer, all being devices well known to those skilled in the art.

In one embodiment, the pharmaceutical composition of the invention may further be compounded in, or attached to, for example through covalent, hydrophobic and electrostatic interactions, a drug carrier, drug delivery system and advanced drug delivery system in order to further enhance stability of the GH conjugate, increase bioavailability, increase solubility, decrease adverse effects, achieve chronotherapy well known to those skilled in the art, and increase patient compliance or any combination thereof. Examples of carriers, drug delivery systems and advanced drug delivery systems include, but are not limited to, polymers, for example cellulose and derivatives, polysaccharides, for example dextran and derivatives, starch and derivatives, chitosans and derivatives, poly(vinyl alcohol), acrylate and methacrylate polymers, polylactic and polyglycolic acid and block co-polymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, L2 phase and dispersions thereof, well known to those skilled in the art of phase behavior in lipid-water systems, polymeric micelles, multiple emulsions, self-emulsifying, self-microemulsifying, cyclodextrins and derivatives thereof, and dendrimers.

The various examples of delivery systems for oral formulation incorporated herein by reference include surfactants, which are known to increase the penetration of hydrophilic compounds. Examples of surfactants are; sodium caprate, tartaric acid, Brij56, Brij58, Brij35, Brij30, fatty acid sugars, sodium taurodeoxycholate, sodium dodecyl sulfate, p-t-octyl phenol poloxyethylene-9.5 (Triton X-100) as described by Takatsuka et al., Eur. J. Pharm. Biopharm. (2006) 62, 52-58 The oral delivery system may also include protease inhibitors and mucolytic substances. Examples of protease inhibitors are soybean trypsin inhibitor, aprotinin and chymostatin. Examples of mucolytic substances are dithiotreitol and N-acetyl cysteine. Enhancement of intestinal absorption of poorly absorbed hydrophilic compounds by simultaneous use of mycolytic agent and surfactant. Also the 5-CNAC and similar compounds developed by Emisphere (WO2008/101240, WO2008/11283687, WO2008/027854, WO2008/014430, US2008/0095837).

The oral formulation delivery systems may also include tight junction modulators provide, which function as specific tight junction openers of epithelium cells. These tight junction modulators function either transient or non-transient and interfere with the protein complexes that hold the epithelium cells tightly together (Kondoh et al., Mol Pharmacology (2005) 67, 749-756). Other examples of the delivery system for oral formulation include mucoadhesive agents, for example thiol containing additives (co-formulation) or covalently attached sidechains can increase the adhesion to the mucos layer, chitosan and carbomer molecules, polyacrylates, PEG and its derivatives, (Palmberger et al., Eur. J. Pharm. Biopharm. (2007) 66, 405-412; Leitner, V. M. et al., Eur. J. Pharm. Biopharm. (2003) 56, 207-214; H. L. Leuβen et al., Parm. Res. (1996) 13, 1668-1672; H. L. Leufβen et al., Int. J. Pharmaceuticals (1996) 141, 39-52; Takatsuka et al., Eur. J. Pharm. Biopharm. (2006) 62, 52-58. Additional examples of delivery systems for oral formulation include cavelolar/lipid rafts, SMVT (sodium dependent multi vitamin transporter). Another example of formulations for oral delivery includes receptor-mediated trancytosis such as IRF (intrinsic factor receptor) using Vitamin B12 (Cobalamin) as substrate, FcRn (neonatal Fc receptor) and Transferrin (M. Gumbleton, Adv. Drug. Del. Rev. (2001) 49, 281-300; K. C. Partlow et al., Biomaterials (2008) 29, 3367-3375; Lee et al., Biotechnol. Appl. Biochem. (2007) 46, 211-217; S. Y. Chae et al., Bioconjugate Chem. (2008) 19, 334-341; Russell-Jones G.: Chapter 17 in Membrane Transporters as Drug Targets (1999); Said and Mohammed Curr. Opin. Gastroent. (2006) 22, 140-146; Chalasani et al., J. Con. Release (2007) 117, 421-429; H. Li & Z. M. Qian Med. Res. Rev. (2002) 22, 225-250; Liang & Yang Biochem. Biophys. Res. Comm. (2005) 225, 734-738).

In one embodiment the GH compounds of the present invention exert growth hormone activity and may be used for treating diseases or states which will benefit from an increase in the amount of circulating growth hormone. Such diseases or states include growth hormone deficiency (GHD); Turner Syndrome; Prader-Willi syndrome (PWS); Noonan syndrome; Down syndrome; chronic renal disease, juvenile rheumatoid arthritis; cystic fibrosis, HIV-infection in children receiving HAART treatment (HIV/HALS children); short children born short for gestational age (SGA); short stature in children born with very low birth weight (VLBW) but SGA; skeletal dysplasia; hypochondroplasia; achondroplasia; idiopathic short stature (ISS); GHD in adults; fractures in or of long bones, such as tibia, fibula, femur, humerus, radius, ulna, clavicula, matacarpea, matatarsea, and digit; fractures in or of spongious bones, such as the scull, base of hand, and base of food; patients after tendon or ligament surgery in e.g. hand, knee, or shoulder; patients having or going through distraction oteogenesis; patients after hip or discus replacement, meniscus repair, spinal fusions or prosthesis fixation, such as in the knee, hip, shoulder, elbow, wrist or jaw; patients into which osteosynthesis material, such as nails, screws and plates, have been fixed; patients with non-union or mal-union of fractures; patients after osteatomia, e.g. from tibia or 1st toe; patients after graft implantation; articular cartilage degeneration in knee caused by trauma or arthritis; osteoporosis in patients with Turner syndrome; osteoporosis in men; adult patients in chronic dialysis (APCD); malnutritional associated cardiovascular disease in APCD; reversal of cachexia in APCD; cancer in APCD; chronic abstractive pulmonal disease in APCD; HIV in APCD; elderly with APCD; chronic liver disease in APCD, fatigue syndrome in APCD; Chron's disease; impaired liver function; males with HIV infections; short bowel syndrome; central obesity; HIV-associated lipodystrophy syndrome (HALS); male infertility; patients after major elective surgery, alcohol/drug detoxification or neurological trauma; aging; frail elderly; osteo-arthritis; traumatically damaged cartilage; erectile dysfunction; fibromyalgia; memory disorders; depression; traumatic brain injury; subarachnoid haemorrhage; very low birth weight; metabolic syndrome; glucocorticoid myopathy; or short stature due to glucocorticoid treatment in children. Growth hormones have also been used for acceleration of the healing of muscle tissue, nervous tissue or wounds; the acceleration or improvement of blood flow to damaged tissue; or the decrease of infection rate in damaged tissue.

In one embodiment, the present invention relates to a method of treating diseases, wherein growth hormone compound activity maybe used for treating diseases or states which will benefit from an increase in the amount of circulating growth hormone compound said method comprising administering to a patient an effective amount of a pharmaceutical composition of growth hormone compound or its conjugate of SEQ ID No.1.

In one embodiment, the present invention relates to a method comprising administration to a patient in need thereof an effective amount of a therapeutically effective amount of growth hormone compound according to the invention. The present invention thus provides a method for treating these diseases or states, the method comprising administering to a patient in need thereof a therapeutically effective amount of a growth hormone compound according to the present invention.

A “therapeutically effective amount” of a compound of the invention as used herein means an amount sufficient to cure, alleviate or partially arrest the clinical manifestations of a given disease and its complications. An amount adequate to accomplish this is defined as “therapeutically effective amount”. Effective amounts for each purpose will depend on e.g. the severity of the disease or injury as well as the weight, sex, age and general state of the subject. It will be understood that determining an appropriate dosage may be achieved using routine experimentation, which is all within the ordinary skills of a trained physician or veterinary.

In one embodiment, the invention provides the use of a growth hormone compound or its conjugate in the manufacture of a medicament used in the treatment of the above mentioned diseases or states.

The growth hormone compounds as defined and described herein in the present invention are intended to be used as a therapeutic protein.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law

The invention is further described by the non limiting list of embodiments presented here below.

### Embodiment 1

A growth hormone conjugate of formula (I)

A-W—B-GH (I)

wherein
GH represents a growth hormone compound,
A represents a bile acid residue,
B represents a hydrophilic spacer covalently linked to GH,
W is a chemical group linking A and B;
and pharmaceutically acceptable salt.

### Embodiment 2

The growth hormone conjugate according to embodiment 1, wherein the growth hormone compound GH comprises one or more additional disulfide bond(s) compared to human growth hormone (hGH) as defined by SEQ ID NO 1.

### Embodiment 3

The growth hormone conjugate according to embodiment 2, wherein the growth hormone compound GH comprises one or more additional disulfide bond(s) connecting a loop segment and a helical segment.

### Embodiment 4

The growth hormone conjugate according to any of the preceding embodiments, wherein the growth hormone compound comprise at least one pair of mutations corresponding to R16C/L1170, A17C/E174C, H21C/M170C, D26/V102C, D26/Y103C, N47C/T50C, Q49C/G161C, F54C/Y143C, F54C/S144C, F54C/F146C, S55C/Y143C, S57C/Y143C, 158C/Q141C, 158C/Y143C, 158C/S144C, P59C/Q137C, P61 C/E66C, P61C/T67C, S71C/S132C, L73C/S132C, L73C/F139C, R77C/I138C, R77C/F139C, L81C/Q141C, L81C/Y143C, Q84C/Y143C, Q84C/S144C, S85C/Y143C, S85C/S144C, P89C/F146C, F92C/F146C, F92C/T148C, R94C/D107C, V102C/A105C, L156C/F146C, L156C/T148C and/or V185C/S188C in SEQ ID NO 1.

### Embodiment 5

The growth hormone conjugate according to embodiment 4, wherein the growth hormone compound comprises at least one pair of mutations corresponding to A17C/E174C, H21C/M170C, D26/V102C, D26/Y103C, F54C/Y143C, F54C/S144C, F54C/F146C, S55C/Y143C, S57C/Y143C, 158C/Q141C, 158C/Y143C, 158C/S144C, P59C/Q137C, S71C/S132C, L81C/Y143C, Q84C/Y143C, S85C/Y143C, S85C/S144C, F92C/T148C and/or R⁹⁴C/D107C in SEQ ID NO 1.

### Embodiment 6

The growth hormone conjugate according to any of the previous embodiments, wherein the growth hormone compound comprises an additional disulfide bond wherein at least one of the cysteines is present in L3 corresponding to AA 128-154 in SEQ ID NO 1 or such as in a region corresponding to AA 135-148 in SEQ ID NO 1.

### Embodiment 7

The growth hormone conjugate according to embodiment 6, wherein at least one of the cysteines of the additional disulfide bond is present in L3 in a position corresponding to AA 141, AA142, AA143, AA144, AA145 or AA146, preferably AA143 or AA144 in SEQ ID NO 1.

### Embodiment 8

The growth hormone conjugate according to embodiment 6, wherein the growth hormone compound comprises at least one pair of mutations corresponding to F54C/Y143C, F54C/S144C, F54C/F146C, S55C/Y143C, S57C/Y143C, 158C/Q141C, 158C/Y143C, 158C/S144C, P59C/Q137C, S71C/S132C, L73C/S132C, L73C/F139C, R77C/I138C, R77C/F139C, L81C/Q141C, L81C/Y143C, Q84C/Y143C, Q84C/S144C, S85C/Y143C, S85C/S144C, P89C/F146C, F92C/F146C and/or F92C/T148C in SEQ ID NO 1.

### Embodiment 9

The growth hormone conjugate according to embodiment 8, wherein the growth hormone compound comprises at least one pair of mutations corresponding to F54C/Y143C, F54C/S144C, F54C/F146C, S55C/Y143C, S57C/Y143C, 158C/Q141C, 158C/Y143C, 158C/S144C, P59C/Q137C, S71C/S132C, L81C/Y143C, Q84C/Y143C, S85C/Y143C, S85C/S144C and/or F92C/T148C in SEQ ID NO 1.

### Embodiment 10

The growth hormone conjugates according to any of the previous claims, wherein the growth hormone compound comprises an additional disulfide bond connecting L3 with L1.

### Embodiment 11

The growth hormone conjugate according to embodiment 10, wherein the compound comprises an additional disulfide bond connecting an amino acid residue corresponding to AA54, AA55, AA56, AA57, AA58 or AA59 in L3 with an amino acid corresponding to AA143 or AA144 in L1 of SEQ ID NO 1.

### Embodiment 12

The growth hormone conjugate according to embodiment 11, wherein the compound comprises at least one pair of mutations corresponding to F54C/Y143C, F54C/S144C, F54C/F146C, S55C/Y143C, S57C/Y143C, 158C/Q141C, 158C/Y143C, 158C/S144C, P59C/Q137C and/or S71C/S132C in SEQ ID NO 1.

### Embodiment 13

The growth hormone conjugate according to any of embodiments 2-9, wherein the growth hormone compound comprises an additional disulfide bond connecting L3 with a helical segment.

### Embodiment 14

The growth hormone conjugate according to embodiment 13, wherein the growth hormone compound comprises an additional disulfide bond connecting L3 with helix 2.

### Embodiment 15

The growth hormone conjugate according to embodiment 14, wherein the compound comprises an additional disulfide bond connecting an amino acid residue corresponding to AA84 or AA85 in H2 with an amino acid corresponding to AA143 or AA144 in L3 of SEQ ID NO 1.

### Embodiment 16

The growth hormone conjugate according to embodiment 14, wherein the compound comprises at least one pair of mutations corresponding to L73C/S132C, L73C/F139C, R77C/1138C, R77C/F139C, L81C/Q141C, L81C/Y143C, Q84C/Y143C, Q84C/S144C, S85C/Y143C, S85C/S144C, P89C/F146C, F92C/F146C and F92C/T148C in SEQ ID No.1.

### Embodiment 17

The growth hormone conjugate according to embodiment 16, wherein the compound comprises at least one pair of mutations corresponding to L81C/Y143C, Q84C/Y143C, S85C/Y143C, S85C/S144C and/or F92C/T148C in SEQ ID NO 1.

### Embodiment 18

The growth hormone conjugate according to any of embodiments 1-9, wherein the growth hormone compound comprises an additional disulfide bond connecting L2 with helix 1.

### Embodiment 19

The growth hormone conjugate according to embodiment 18, wherein the compound comprises at least one pair of mutations corresponding to D26C/V102C or D26C/Y103C.

### Embodiment 20

The growth hormone conjugate according to any of the preceding embodiments, wherein the polypeptide sequence is at least 80%, such as 90%, such as 95%, such as 96%, such as 97%, such as 98% or such as 99% identical to hGH defined by SEQ ID NO 1.

### Embodiment 21

The growth hormone conjugate according to any of the preceding embodiments, wherein the growth hormone compound comprises at least one single point mutation is at a protease site.

### Embodiment 22

The growth hormone conjugate according to any of the preceding embodiments, wherein the growth hormone compound comprise at least one single point mutation at a position(s) corresponding to position 1-55, 57, 58, 60-63, 67-87, 89-91, 93, 95-100, 102-128, 131-132, 135-139, 141, 142, 144, 148-182, 184, 185 and/or 187-191 of SEQ ID NO 1.

### Embodiment 23

The growth hormone conjugate according to any of the preceding embodiments, wherein the growth hormone compound comprise at least one single point mutation at a position(s) corresponding to position position 55, 57, 62, 101, 134, 136, 142 and/or 144 of SEQ ID NO 1

### Embodiment 24

The growth hormone conjugate according to any of the preceding embodiments, wherein at least one single point mutation is present in L1 (corresponding to AA 36-71 of SEQ ID NO 1) and/or L3 (corresponding to AA 128-154 of SEQ ID NO 1).

### Embodiment 25

The growth hormone conjugate according to Embodiment 24, wherein at least one single point mutation is present in L1.

### Embodiment 26

The growth hormone conjugate according to Embodiment 25, wherein at least one single point mutation is present in the middle region of L1 (corresponding to AA 40-65 of SEQ ID NO 1).

### Embodiment 27

The growth hormone conjugate according to Embodiment 26, wherein at least one single point mutation is present in a position corresponding to position 40, 41, 42 and/or 62 of SEQ ID NO 1.

### Embodiment 28

The growth hormone conjugate according to Embodiment 24, wherein at least one single point mutation is present in L3.

### Embodiment 29

The growth hormone conjugate according to Embodiments 28, wherein at least one single point mutation is present in the middle region of L3 (corresponding to AA 135-148 of SEQ ID NO 1).

### Embodiment 30

The growth hormone conjugate according to Embodiment 29, wherein at least one single point mutation is in a position corresponding to position 134, 136, 139, 142 and/or 144 of SEQ ID NO 1.

### Embodiment 31

The growth hormone conjugate according to any of the preceding embodiments, wherein the in vitro activity for said compound is at least 5% if the activity of wild type hGH defined by SEQ ID NO 1.

### Embodiment 32

The growth hormone conjugate according to any of the previous embodiments, wherein the functional in vivo half-life of the polypeptide is 2 times or more compared to human growth hormone.

### Embodiment 33

The growth hormone conjugate according to any of the previous embodiments, wherein the functional in vivo half-life of the polypeptide is between 2 and 10 times more compared to human growth hormone.

### Embodiment 34

The growth hormone conjugate according to any of the preceding embodiments, wherein the growth hormone conjugate is stabilized towards degradation by protease(s), such as digestive proteases, such as pepsin, trypsin, chymotrypsin, carboxypeptidase and/or elastases.

### Embodiment 35

The growth hormone conjugate according to embodiment 34, wherein the compound is stabilized towards degradation by Chymotrypsin and/or Elastase.

### Embodiment 36

The growth hormone conjugate according to embodiment 35, wherein the growth hormone compound comprises at least one pair of mutations corresponding to H21C/M170C, D26C/V102C, D26C/Y103C, F54C/Y143C, F54C/S144C, S55C/Y143C, S57C/Y143C, 158C/Q141C, 158C/Y143C, 158C/S144C, P59C/Q137C, S71C/S132C, L81C/Y143C, Q84C/Y143C, S85C/Y143C and/or S85C/S144C in SEQ ID NO 1.

### Embodiment 37

The growth hormone conjugate according to embodiment 36, wherein the growth hormone compound comprises at least one pair of mutations corresponding to D26CN102C, D26C/Y103C, S57C/Y143C, 158C/S144C, P59C/Q137C, S71C/S132C, Q84C/Y143C, S85C/Y143C, S85C/S144C, F92C/T148C and/or R94C/D107C in SEQ ID NO 1.

### Embodiment 38

The growth hormone conjugate according to embodiment 35, wherein the growth hormone compound comprises at least one pair of mutations corresponding to S57C/Y143C, Q84C/Y143C, S85C/Y143C and/or S85C/S144C in SEQ ID NO 1.

### Embodiment 39

The growth hormone conjugate according to any of the previous embodiments, wherein the one or more additional disulfide bond(s) is/are obtained by amino acid substitution of at least two amino acid compared SEQ ID NO 1.

### Embodiment 42

The growth hormone conjugate according to any of the previous embodiments wherein the growth hormone compound comprises exactly one additional disulfide bond compared to SEQ ID NO 1.

### Embodiment 41

The growth hormone conjugate according to any of the previous embodiments wherein the growth hormone compound comprises at least two additional cysteines compared to human growth hormone as defined in SEQ ID NO 1.

### Embodiment 42

The growth hormone conjugate according to any of the previous embodiments wherein the compound comprises exactly 3 amino acid substitutions compared to SEQ ID NO 1.

### Embodiment 43

The growth hormone conjugate according to any of the previous embodiments comprising exactly two additional cysteines and exactly one single point mutation compared to human growth hormone as defined in SEQ ID NO 1.

### Embodiment 44

The growth hormone conjugate according to any of the previous embodiments wherein the growth hormone compound comprises exactly two additional cysteines compared to human growth hormone as defined in SEQ ID NO 1.

### Embodiment 45

The growth hormone conjugate according to any of the embodiments 1-43, wherein the growth hormone compound comprises exactly three additional cysteines compared to human growth hormone as defined in SEQ ID NO 1.

### Embodiment 46

The growth hormone conjugate according to embodiment 45, wherein the growth hormone compound comprises one additional disulphide bond and a single cys mutation compared to human growth hormone as defined in SEQ ID NO 1.

### Embodiment 47

The growth hormone conjugate according to embodiment 46, wherein the single cys mutation is selected from any one of a single cys mutation in the N-terminal, H1, H2, L2 or H3 regions of GH.

### Embodiment 48

The growth hormone conjugate according to embodiment 46, wherein the single cys mutation is selected from the group of mutation corresponding to: T3C, P5C, S7C, D110, H180, Q29C, E30C, E33C, A340, Y35C, Q40C, S55C, S57C, S62C, E88C, Q91C, S95C, A98C, N99C, S100C, L101C, V102C, Y103C, D107C, S108C, D112C, Q122C and G126C in hGH (SEQ ID NO:1).

### Embodiment 49

The growth hormone conjugate according to embodiment 47, wherein the single cys mutation is located in a position corresponding to AA 93-106, such as AA 99-106 or AA 99-103 in hGH.

### Embodiment 50

The growth hormone conjugate according to any of the previous embodiments, wherein GH is conjugated at a wild type amino acid residue.

### Embodiment 51

The growth hormone conjugate according to any of the previous embodiments, wherein GH is conjugated at a wild type amino acid residue selected from the group of: the N-terminal, the C-terminal, Gln40 and Gln141.

### Embodiment 52

The growth hormone conjugate according to any of the previous embodiments 1-50, wherein GH is conjugated at a mutant residue.

### Embodiment 53

The growth hormone conjugate according to embodiment 52, wherein the mutant residue is a cys mutation.

### Embodiment 54

The growth hormone conjugate according to embodiment 53, wherein the cys mutation is selected from the group of mutation corresponding to: T3C, P5C, S7C, D110, H180, Q29C, E30C, E33C, A34C, Y35C, Q40C, S55C, S57C, S62C, E88C, Q91C, S95C, A98C, N99C, S100C, L101C, V102C, Y103C, D107C, S108C, D112C, Q122C and G126C in hGH (SEQ ID NO:1).

### Embodiment 55

The growth hormone conjugate according to embodiment 54, wherein the cys mutant correspond to a L101C mutation of human growth hormone.

### Embodiment 56

The growth hormone conjugate according to any of embodiments 1-55, wherein the growth hormone conjugate is stabilized towards proteolytic degradation by protease(s), such as digestive proteases, such as pepsin, trypsin, chymotrypsin, carboxypeptidase and/or elastases compared to human growth hormone.

### Embodiment 57

The growth hormone conjugate according to any of embodiments 1-56, wherein the growth hormone compound is chemically modified in order to facilitate transport across the epithelia.

### Embodiment 58

The growth hormone conjugate according to any of embodiments 1-58, wherein the growth hormone compound is chemically modified in order to facilitate transport across the epithelia when compared to wt hGH.

### Embodiment 59

The growth hormone conjugate according to any of embodiments 1-58, wherein the growth hormone compound is chemically modified in order to obtain a prolonged functional in vivo half-life when compared to wt hGH.

### Embodiment 60

The growth hormone conjugate according to embodiment 59, wherein the functional in vivo half-life of said growth hormone compound is 2 times or more compared to hGH.

### Embodiment 61

The growth hormone conjugate according to embodiment 60, wherein the functional in vivo half-life is between 2 and 10 times compared to hGH.

### Embodiment 62

The growth hormone conjugate according to any embodiments 57-61, wherein the chemical modification takes place at amino acid residues not interfering with binding of the growth hormone compound to the hGHR.

### Embodiment 63

The growth hormone conjugate according to any of the previous embodiments, wherein the bile acid residue (A) is selected from the group consisting of: cholanic acid, cholic acid, chenodeoxycholic acid, deoxycholic acid, ursodeoxycholic acid, lithocholic acid, glycocholate, glycodeoxycholate, glycochenodeoxycholate, taurocholate, taurodeoxycholate and taurochenodeoxycholate.

### Embodiment 64

The growth hormone conjugate according to any of the previous embodiments, wherein the bile acid residue (A) is selected from the group consisting of:

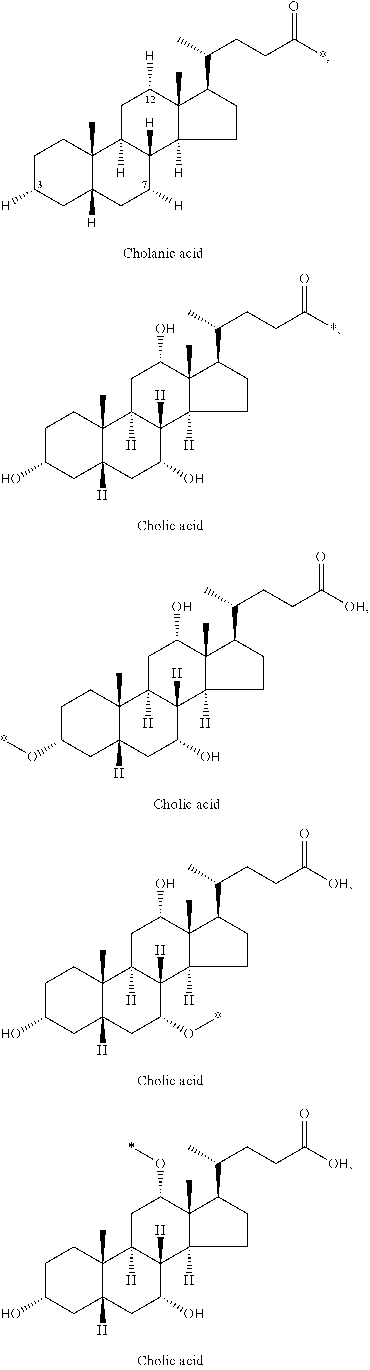

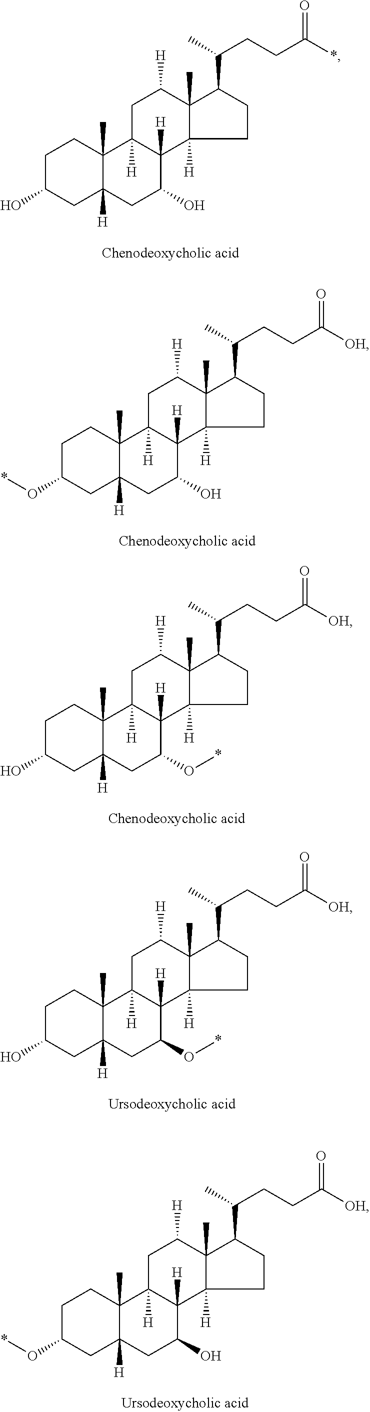

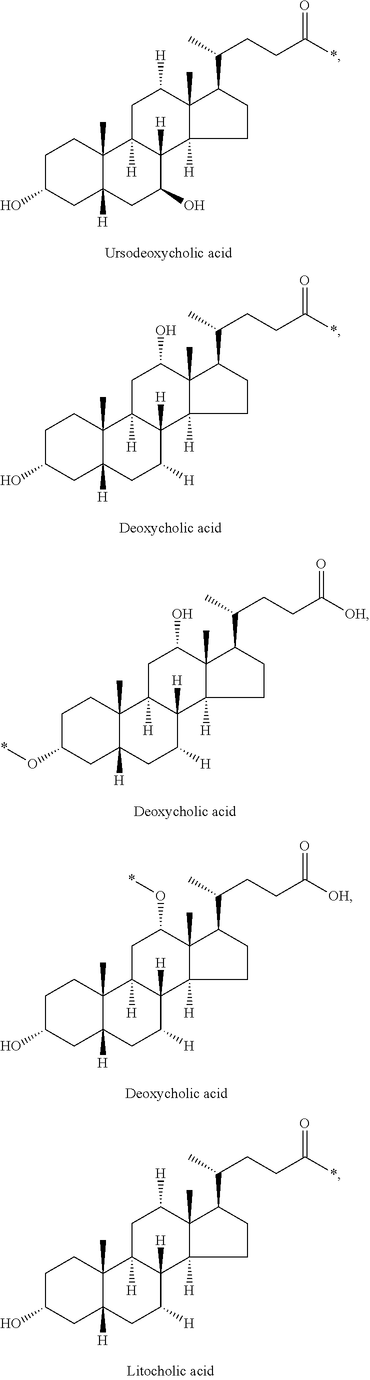

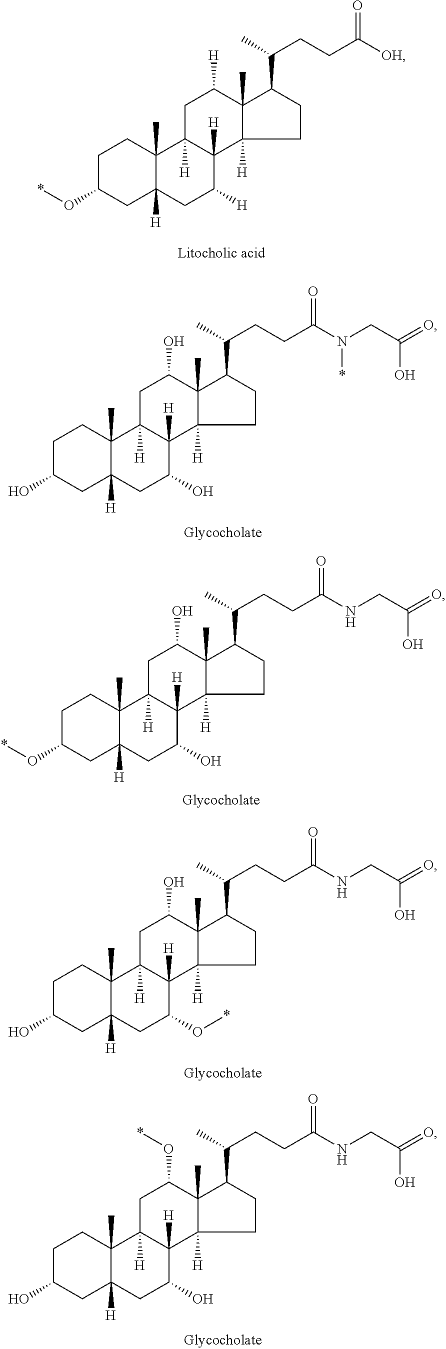

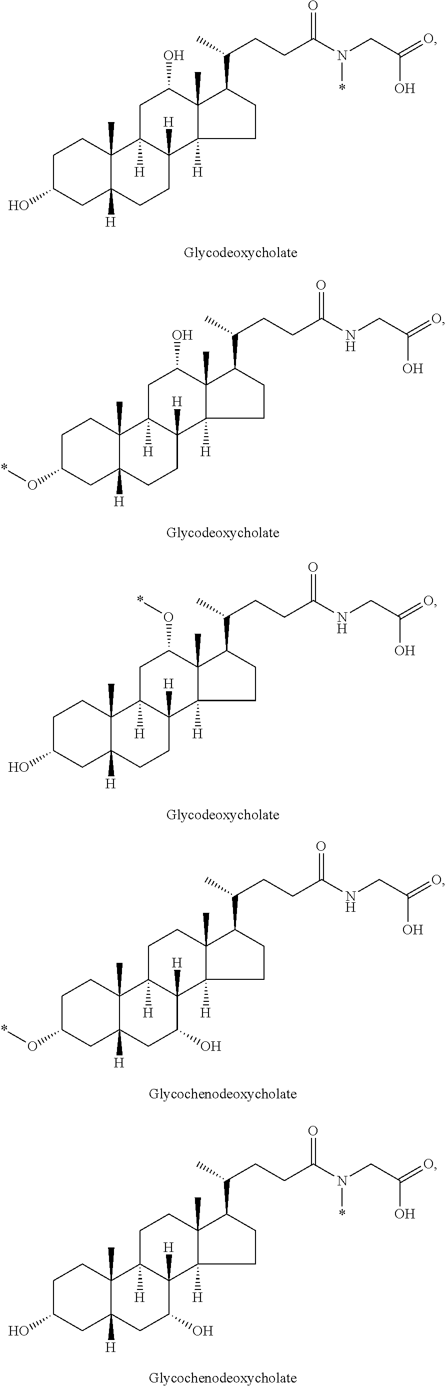

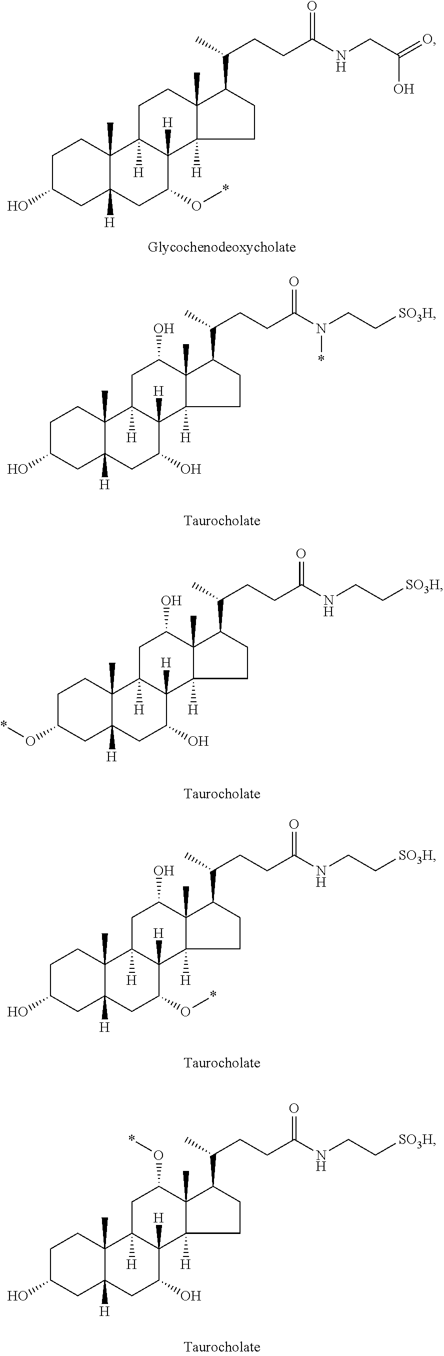

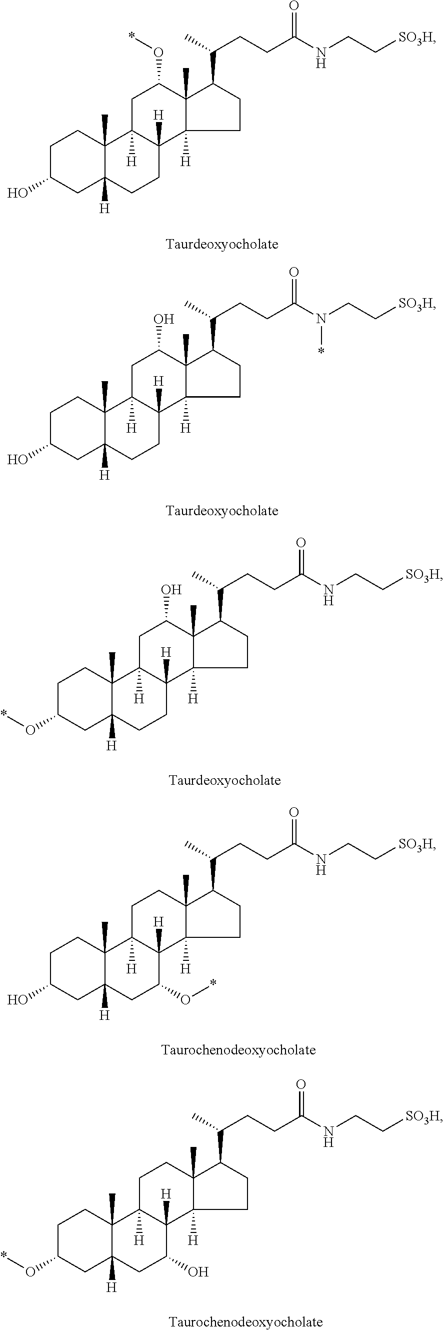

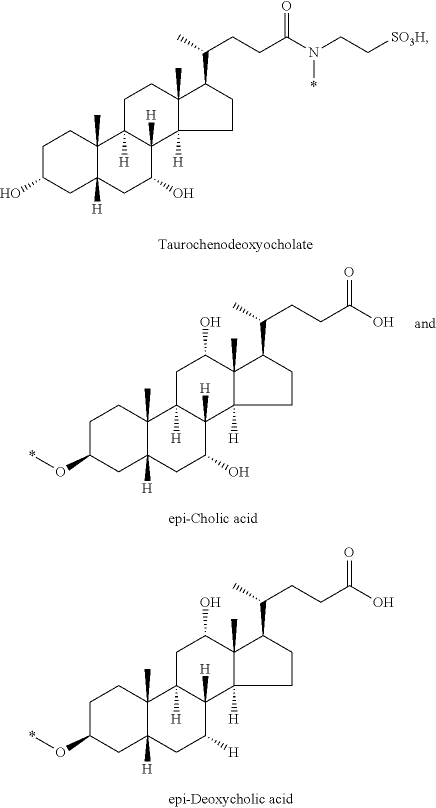

wherein (*) represents the attachment point to the hydrophilic spacer (B) through W.

### Embodiment 65

The growth hormone conjugate according to any of the previous embodiments, wherein the bile acid residue (A) is a cholic acid residue.

### Embodiment 66

The growth hormone conjugate of embodiment 65, wherein the cholic acid residue is selected from

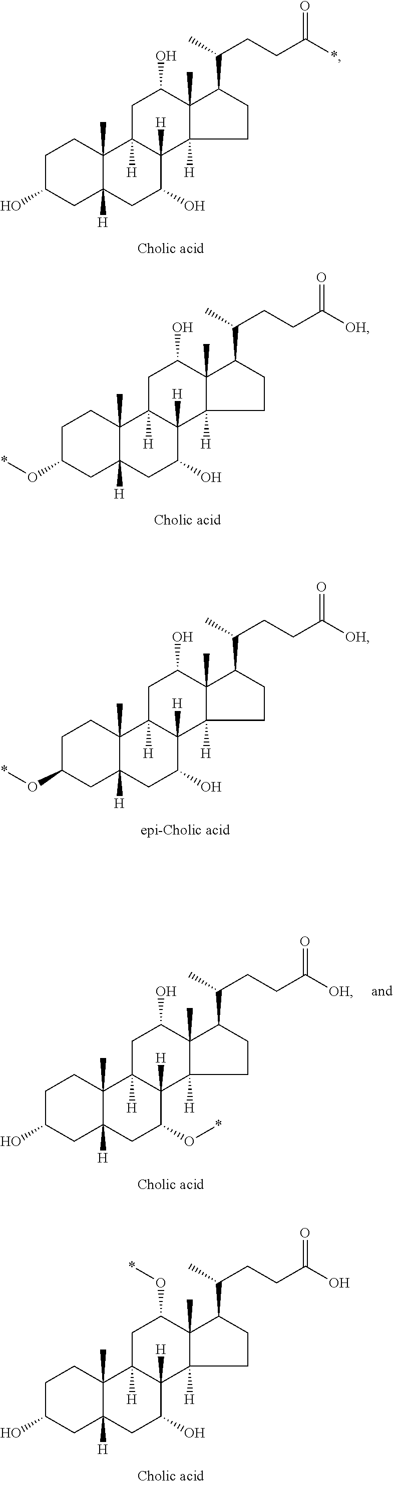

wherein (*) represents the attachment point to the hydrophilic spacer (B) through W.

### Embodiment 67

The growth hormone conjugate according to Embodiment 63, wherein the cholic acid residue is linked to the hydrophilic spacer (B) through W via position “3”, “7”, “12” or “24”.

### Embodiment 68

The growth hormone conjugate of any of the previous embodiment, wherein the chemical group (W) is selected from: —NR¹—, —NHC(O)[CH₂]ₙ₁C(O)—, —NHC(O)CH═CHC(O)—, —C(O)NH—, —NHC(O)—, —C(O)NHCH₂—, —CH₂NHC(O)—, —C(O)NHS(O)₂—, —S(O)₂NHC(O)—, —NHC(O)O—, —C(O)CH₂—, —CH₂C(O)—, —C(O)CH═CH—, —CH═CHC(O)—, or —C(O)—;

wherein R¹ is selected from hydrogen or -[CH₂]ₙ₂R², n2 is 1 to 4 and R² is selected from −C(O)OH, —S(O)₂OH or tetrazol-1-yl and wherein n1 is 2 to 8.

### Embodiment 69

The conjugate according to any of the previous embodiments wherein the hydrophilic spacer (B) has the formula

—X₁—X₂—X₃—X₄

wherein
X₁ is —W₁—[(CHR³)ₗ₁—W₂]ₘ₁-{[(CH₂)ₙ₁E1]ₘ₂—[(CHR⁴)ₗ₂—W₃]ₘ₃}ₙ₂-,
X₂ is —[(CHR⁵)ₗ₃—W₄]ₘ₄-{[(CH₂)ₙ₃E2]ₘ₅—[(CHR⁶)ₗ₄—W₅]ₘ₆}ₙ₄-,
X₃ is —[(CHR⁷)ₗ₅—W₆]ₘ₇-,
X₄ is F-D1-(CH₂)ₗ₆-D2-,
l1, l2, l3, l4, l5 and l6 independently are selected from 0-10,
m1, m3, m4, m6 and m7 independently are selected from 0-6,
m2 and m5 independently are selected from 0-6,
n1, n2, n3 and n4 independently are selected from 0-6,
F is aryl, hetaryl, pyrrolidine-2,5-dione or a valence bond, wherein the aryl and
hetaryl groups are optionally substituted with halogen, —CN, —OH, —C(O)OH, —C(O)NH₂, —S(O)₂OH or C₁₋₆-alkyl,
R³, R⁴, R⁵, R⁶ and R⁷ independently are selected from hydrogen, —C(O)OH, —C(O)NH₂, —S(O)OH, —S(O)₂OH, —NH—C(═NH)—NH₂, C₁₋₆-alkyl, aryl or hetaryl;
wherein
the alkyl, aryl and hetaryl groups optionally are substituted with halogen, —C(O)OH, —C(O)NH₂, —S(O)OH, —S(O)₂OH, —CN or —OH,
D1, D2, E1 and E2 independently are selected from —O—, —N(R⁸)—, —N(C(O)R⁹)— or a valence bond; wherein R⁸ and R⁹ independently represent hydrogen or C₁₋₆-alkyl, W₁ to W₆ independently are selected from —NH—, —C(O)NH—, —NHC(O)—, —C(O)NHCH₂—, —CH₂NHC(O)—, —C(O)NHS(O)₂—, —S(O)₂NHC(O)—, —OC(O)NH—, —NHC(O)O—, —C(O)CH₂—, —NHC(O)C₁₋₆-alkyl, —C(O)NHC₁₋₆-alkyl, —CH₂C(O)—, —C(O)CH═CH—, —CH═CHC(O)—, —(CH₂)ₛ₂—, —C(O)—, or a valence bond; wherein s2 is 0 or 1.

### Embodiment 70

The conjugate according to embodiment 69, wherein

l1, l2, l3, l4, l5 and l6 independently are 0-6,

m1, m3, m4, m6 and m7 independently are 0-6,

m2 and m5 independently are 0-6, and

n1, n2, n3 and n4 independently are 0-6.

### Embodiment 71

The conjugate according to embodiment 69 or 70, wherein D1 and D2 are independently selected from —O— or a valence bond.

### Embodiment 72

The conjugate according to any of embodiments 69-71, wherein E1 and E2 are independently selected from —O— or a valence bond.

### Embodiment 73

The conjugate according to any of the embodiments 69-72, wherein W₁ through W₆ independently are selected from the group consisting of: —NH—, —C(O)NH—, —NHC(O)—, —CH₂NHC(O)—, —C(O)NHS(O)₂—, —S(O)₂NHC(O)—, —NHC(O)C₁₋₆-alkyl or —C(O)NHC₁₋₆-alkyl and a valence bond.

### Embodiment 74

The conjugate according to any of the embodiments 69-73,
wherein R³, R⁴, R⁵, R⁶ and R⁷ independently are selected from hydrogen, —C(O)OH, —C(O)NH₂, —S(O)₂OH or C₁₋₆-alkyl; wherein the alkyl group optionally is substituted with —C(O)OH, —C(O)NH₂ or —S(O)₂OH.

### Embodiment 75

The growth hormone conjugate according to any of the previous embodiments, wherein the hydrophilic spacer (B) has a solubility (mLogP) below 1.

### Embodiment 76

The growth hormone conjugate according to any of the previous embodiments, wherein the hydrophilic spacer (B) comprises at least one OEG motif (—NH—(CH₂)₂—O—(CH₂)₂—O—CH₂—CO—).

### Embodiment 77

The growth hormone conjugate according to any of the previous embodiments, wherein the hydrophilic spacer (B) comprises at least two OEG motif (—NH—(CH₂)₂—O—(CH₂)₂—O—CH₂—CO—).

### Embodiment 78

The growth hormone conjugate according to any of the previous embodiments, wherein the hydrophilic spacer (B) comprises at lease one glutamic acid residue.

### Embodiment 79

The growth hormone conjugate according to any of the previous embodiments, wherein the hydrophilic spacer (B) comprises at least one glutamic acid residue.

### Embodiment 80

The growth hormone conjugate according to any of the previous embodiments, wherein the hydrophilic spacer (B) comprises at least one lysine residue.

### Embodiment 81

The growth hormone conjugate according to any of the previous embodiments, wherein the hydrophilic spacer (B) comprises at least one glutamic acid residue forming an amide bond via either the alfa or the carboxy group with an amine group from a lysine, an OEG motif or another Glu residue.

### Embodiment 81

The growth hormone conjugate according to any of the previous embodiments, wherein the hydrophilic spacer (B) comprises at least one glutamic acid residue forming an amide bond through the amino group with a carboxy group of the albumin binding residue (A), an carboxy group of an OEG motif, or an gamma- or alfa-carboxy group of another Glu.

### Embodiment 82

The growth hormone conjugate according to any of the previous embodiments, wherein the hydrophilic spacer (B) is selected from:

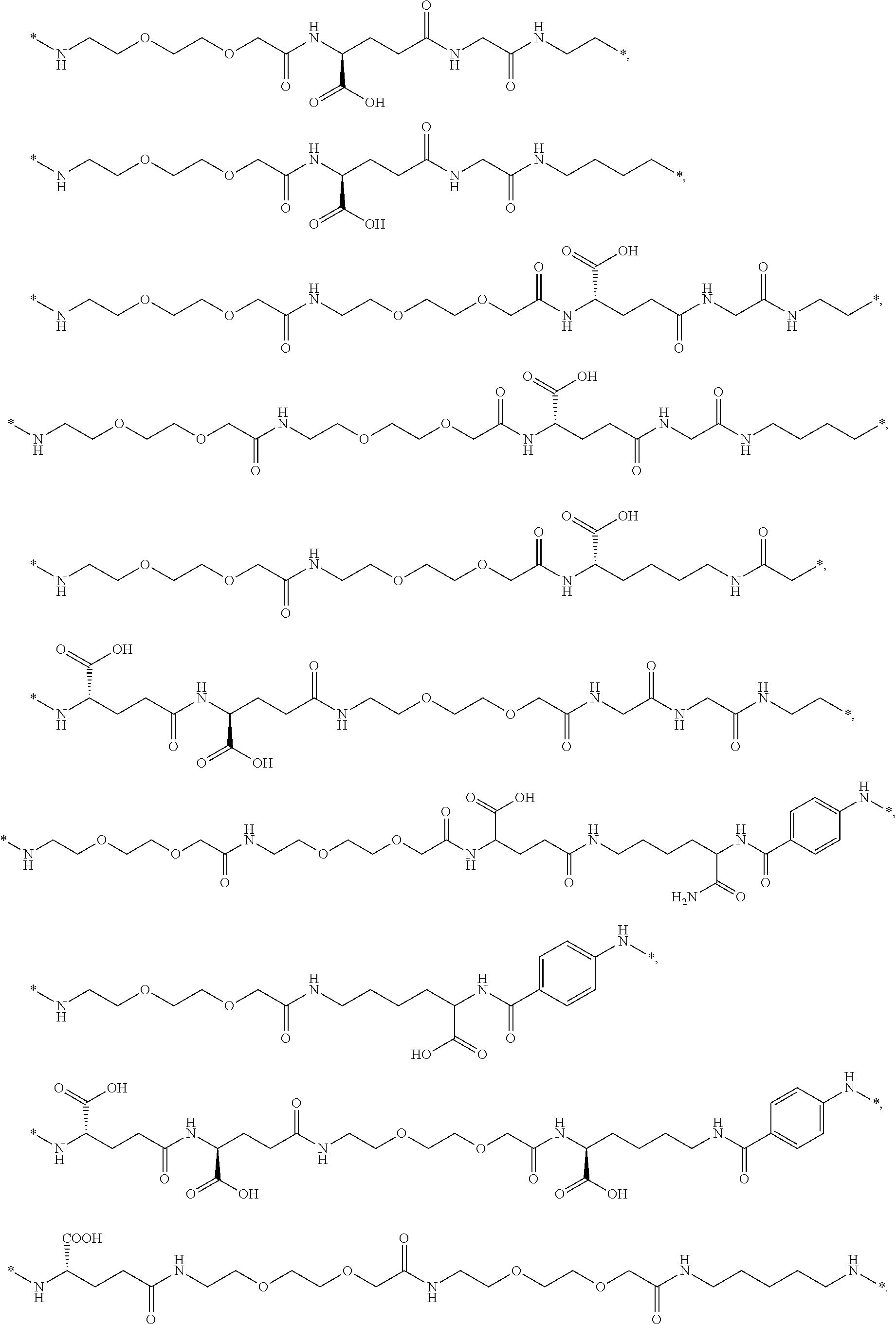

### Embodiment 83

The growth hormone conjugate according to any of the previous embodiments, wherein said conjugate is selected from:

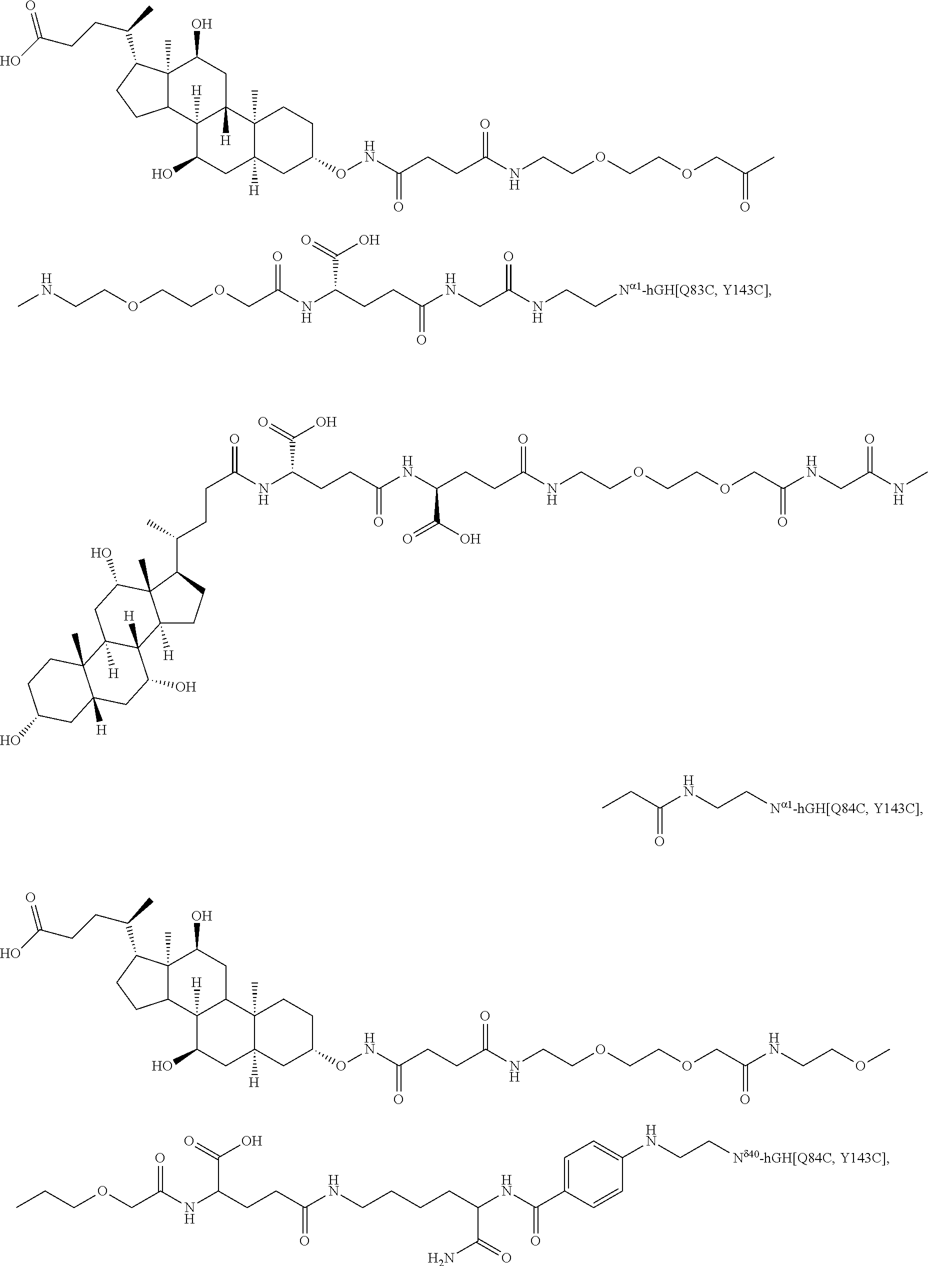

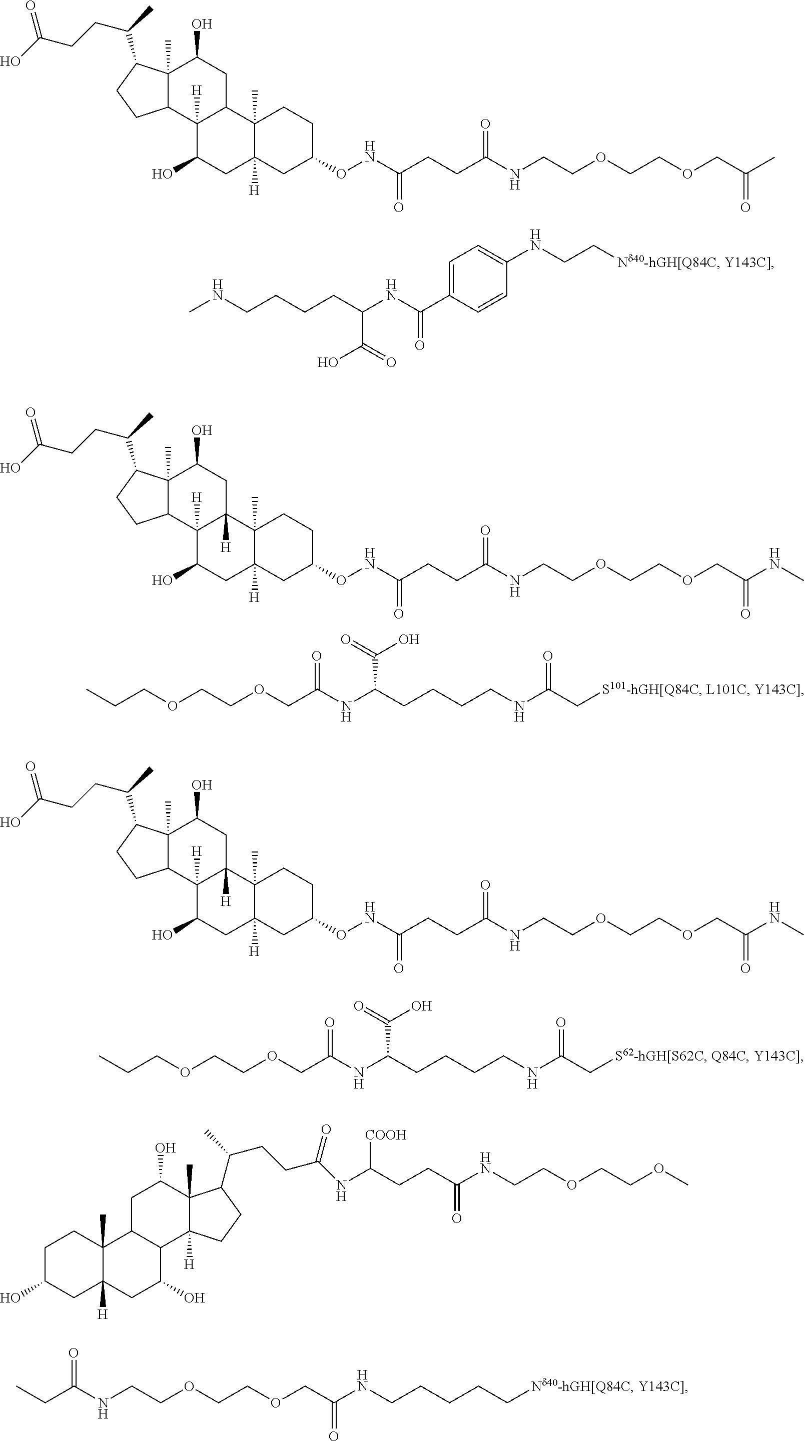

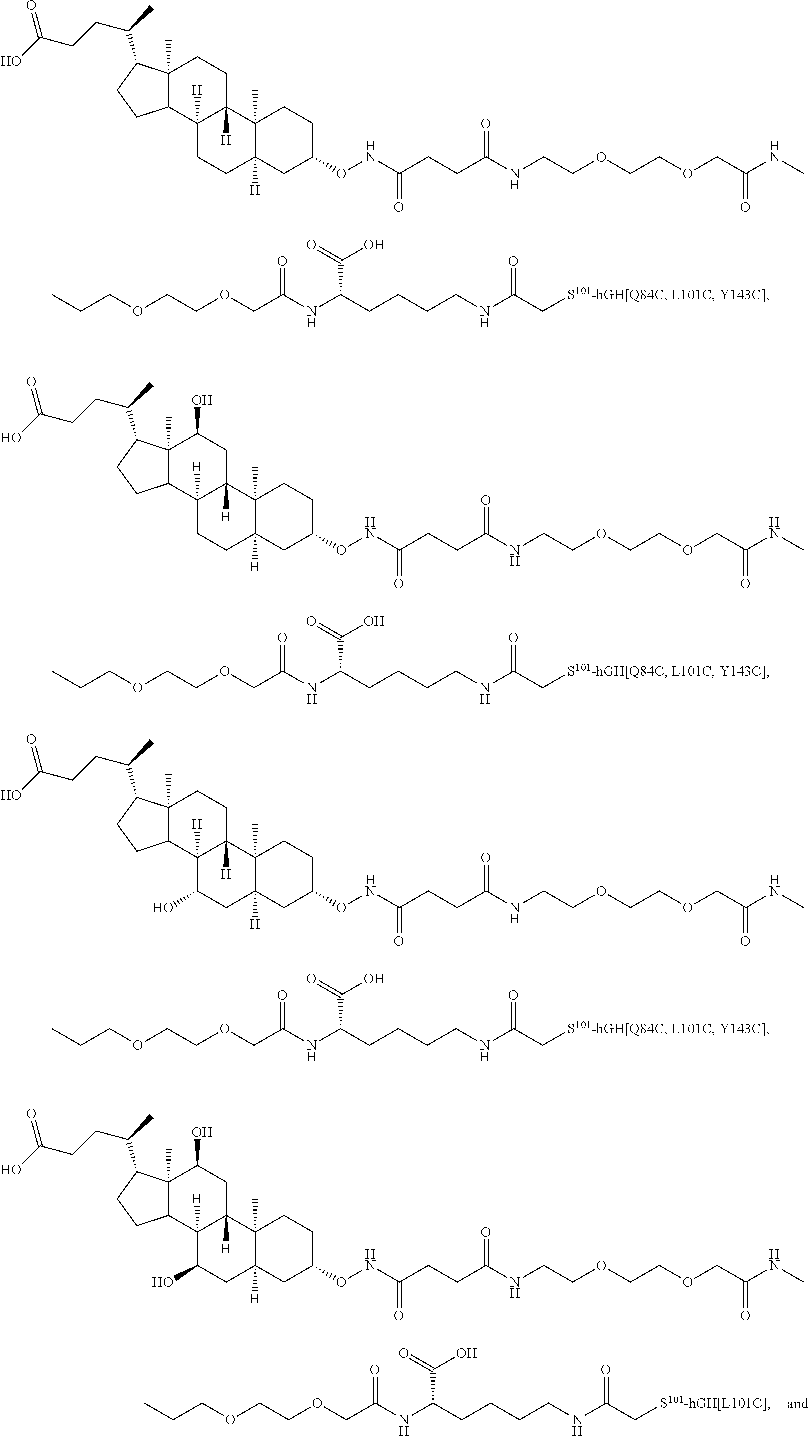

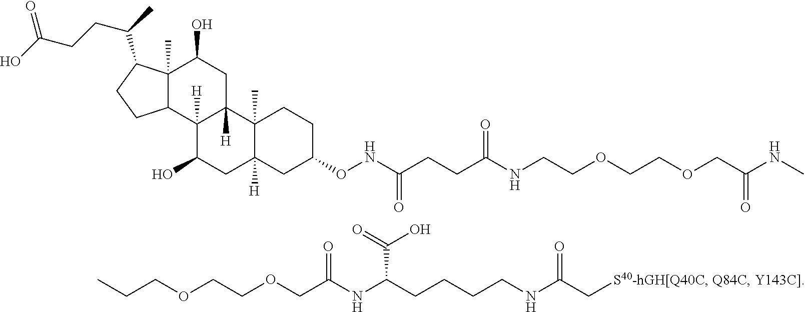

### Embodiment 84

A method for preparing a growth hormone conjugate according to embodiment 1, which method comprises the step of:

a) conjugating a bile acid linker to a growth hormone compound (GH) and
b) obtaining a growth hormone conjugate according to any of the previous embodiments.

### Embodiment 85

The method according to embodiment 84, wherein the bile acid linker is selected from the group of:

A-W—B1-NH₂,A-W—B1-CHO,A-W—B1-LG,A-W—B1-C(O)NHCH₂—CH═CH₂ and

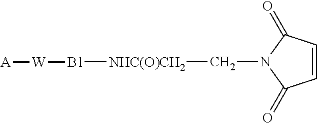

wherein A represents a bile acid residue, B1 represents a hydrophilic spacer, and W is a chemical group linking A and B.

### Embodiment 86

The method according to embodiment 85, wherein the hydrophilic spacer B1 comprises any of the features of the hydrophilic spacer B as defined in embodiments 70-82 or wherein the hydrophilic spacer B1 is comprised by the hydrophilic spacer B as defined in embodiments 70-82.

### Embodiment 87

The method according to embodiment 85, wherein the hydrophilic spacer hydrophilic spacer B1 has the formula: —X₁—X₂—X₃—X₄

wherein
X₁ is —W₁—[(CHR³)ₗ₁—W₂]ₘ₁-{[(CH₂)ₙ₁E1]ₘ₂—[(CHR⁴)ₗ₂—W₃]ₘ₃}ₙ₂-,
X₂ is —[(CHR⁵)ₗ₃—W₄]ₘ₄-{[(CH₂)ₙ₃E2]ₘ₅—[(CHR⁶)ₗ₄—W₅]ₘ₆}ₙ₄-,
X₃ is —[(CHR⁷)ₗ₅]ₘ₇-,
X₄ is a valence bond,
l1, l2, l3, l4, and l5 independently are selected from 0-10,
m1, m3, m4, m6 and m7 independently are selected from 0-6,
m2 and m5 independently are selected from 0-6,
n1, n2, n3 and n4 independently are selected from 0-6,
R³, R⁴, R⁵, R⁶ and R⁷ independently are selected from hydrogen, —C(O)OH,
—C(O)NH₂, —S(O)OH, —S(O)₂OH, —NH—C(═NH)—NH₂, C₁₋₆-alkyl, aryl or hetaryl;
   wherein the alkyl, aryl and hetaryl groups optionally are substituted with halogen, —C(O)OH, —C(O)NH₂, —S(O)OH, —S(O)₂OH, —CN or —OH,
E1 and E2 independently are selected from —O—, —NR⁸—, —N(COR⁹)— or a valence bond; wherein R⁸ and R⁹ independently represent hydrogen or C₁₋₆-alkyl, W₁ to W₆ independently are selected from —NH—, —C(O)NH—, —NHC(O)—, —C(O)NHCH₂—, —CH₂NHC(O)—, —C(O)NHS(O)₂—, —S(O)₂NHC(O)—, —OC(O)NH—, —NHC(O)O—, —C(O)CH₂—, —CH₂C(O)—, —C(O)CH═CH—, —CH═CHC(O)—, —(CH₂)ₛ₂—, —C(O)—, —C(O)O—, —C(O)—, or a valence bond; wherein s2 is 0 or 1.

### Embodiment 87

A pharmaceutical composition comprising growth hormone conjugate according to any of embodiments 1 to 83 and a pharmaceutically acceptable carrier/s.

### Embodiment 88

A pharmaceutical composition according to embodiment 87, wherein said composition can be administered through lingual, sublingual, buccal, in the mouth, oral, in the stomach and intestine, nasal, pulmonary, epidermal, dermal, transdermal, and parenteral to patients.

### Embodiment 89

A pharmaceutical composition according to embodiment 87 or 88, wherein said composition is for oral administration.

### Embodiment 90

A method of preparing a pharmaceutical composition wherein said composition comprising of growth hormone conjugate according to any of embodiments 1 to 83 and pharmaceutically acceptable carrier(s).

### Embodiment 91

A method of treating a disease wherein growth hormone activity may be used for treating diseases or states where the patient will benefit from an increase in the amount of circulating growth hormone said method comprising administering to patient an effective amount of the growth hormone conjugate according to any of embodiments 1 to 83 or a pharmaceutical composition according to any of the embodiments 87-89.

### Embodiment 92

A method of treating diseases or states wherein the patient will benefit from an increase in growth hormone activity said method comprising administering to patient an effective amount of an growth hormone conjugate according to any of embodiments 1 to 83 or a pharmaceutical composition according to any of embodiments 87-89.

### Embodiment 93

A method of treating diseases according to embodiments 90 or 91, wherein the disease is selected from growth hormone deficiency (GHD); Turner Syndrome; Prader-Willi syndrome (PWS); Noonan syndrome; Down syndrome; chronic renal disease, juvenile rheumatoid arthritis; cystic fibrosis, HIV-infection in children receiving HAART treatment (HIV/HALS children); short children born short for gestational age (SGA); short stature in children born with very low birth weight (VLBW) but SGA; skeletal dysplasia; hypochon-droplasia; achondroplasia; idiopathic short stature (ISS); GHD in adults (GHDA); fractures in or of long bones, such as tibia, fibula, femur, humerus, radius, ulna, clavicula, matacarpea, matatarsea, and digit; fractures in or of spongious bones, such as the scull, base of hand, and base of food; patients after tendon or ligament surgery in e.g. hand, knee, or shoulder; patients having or going through distraction oteogenesis; patients after hip or discus replacement, meniscus repair, spinal fusions or prosthesis fixation, such as in the knee, hip, shoulder, elbow, wrist or jaw; patients into which osteosynthesis material, such as nails, screws and plates, have been fixed; patients with non-union or mal-union of fractures; patients after osteatomia, e.g. from tibia or 1st toe; patients after graft implantation; articular cartilage degeneration in knee caused by trauma or arthritis; osteoporosis in patients with Turner syndrome; osteoporosis in men; adult patients in chronic dialysis (APCD); malnutritional associated cardiovascular disease in APCD; reversal of cachexia in APCD; cancer in APCD; chronic abstractive pulmonal disease in APCD; HIV in APCD; elderly with APCD; chronic liver disease in APCD, fatigue syndrome in APCD; Chron's disease; impaired liver function; males with HIV infections; short bowel syndrome; central obesity; HIV-associated lipodystrophy syndrome (HALS); male infertility; patients after major elective surgery, alcohol/drug detoxification or neurological trauma; aging; frail elderly; osteo-arthritis; traumatically damaged cartilage; erectile dysfunction; fibromyalgia; memory disorders; depression; traumatic brain injury; subarachnoid haemorrhage; very low birth weight; metabolic syndrome; glucocorticoid myopathy; or short stature due to glucocorticoid treatment in children.

### Embodiment 94

A growth hormone conjugate according to any of embodiments 1 to 83 for use as an medicament.

### Embodiment 95

A growth hormone conjugate according to any of embodiments 1 to 83 for use as an medicament for treatment of a disease is selected from: growth hormone deficiency (GHD); Turner Syndrome; Prader-Willi syndrome (PWS); Noonan syndrome; Down syndrome; chronic renal disease, juvenile rheumatoid arthritis; cystic fibrosis, HIV-infection in children receiving HAART treatment (HIV/HALS children); short children born short for gestational age (SGA); short stature in children born with very low birth weight (VLBW) but SGA; skeletal dysplasia; hypochondroplasia; achondroplasia; idiopathic short stature (ISS); GHD in adults (GHDA); fractures in or of long bones, such as tibia, fibula, femur, humerus, radius, ulna, clavicula, matacarpea, matatarsea, and digit; fractures in or of spongious bones, such as the scull, base of hand, and base of food; patients after tendon or ligament surgery in e.g. hand, knee, or shoulder; patients having or going through distraction oteogenesis; patients after hip or discus replacement, meniscus repair, spinal fusions or prosthesis fixation, such as in the knee, hip, shoulder, elbow, wrist or jaw; patients into which osteosynthesis material, such as nails, screws and plates, have been fixed; patients with non-union or mal-union of fractures; patients after osteatomia, e.g. from tibia or 1st toe; patients after graft implantation; articular cartilage degeneration in knee caused by trauma or arthritis; osteoporosis in patients with Turner syndrome; osteoporosis in men; adult patients in chronic dialysis (APCD); malnutritional associated cardiovascular disease in APCD; reversal of cachexia in APCD; cancer in APCD; chronic abstractive pulmonal disease in APCD; HIV in APCD; elderly with APCD; chronic liver disease in APCD, fatigue syndrome in APCD; Chron's disease; impaired liver function; males with HIV infections; short bowel syndrome; central obesity; HIV-associated lipodystrophy syndrome (HALS); male infertility; patients after major elective surgery, alcohol/drug detoxification or neurological trauma; aging; frail elderly; osteo-arthritis; traumatically damaged cartilage; erectile dysfunction; fibromyalgia; memory disorders; depression; traumatic brain injury; subarachnoid haemorrhage; very low birth weight; metabolic syndrome; glucocorticoid myopathy; and short stature due to glucocorticoid treatment in children.

### Embodiment 96

Use of a growth hormone according to any of embodiments 1 to 83 as an medicament.

### Embodiment 97

Use of growth hormone compound according to any of embodiments 1 to 83 in a method of treatment of a disease.

### Embodiment 98

Use according to embodiment 69 or embodiment 70, wherein the disease is selected from growth hormone deficiency (GHD); Turner Syndrome; Prader-Willi syndrome (PWS); Noonan syndrome; Down syndrome; chronic renal disease, juvenile rheumatoid arthritis; cystic fibrosis, HIV-infection in children receiving HAART treatment (HIV/HALS children); short children born short for gestational age (SGA); short stature in children born with very low birth weight (VLBW) but SGA; skeletal dysplasia; hypochondroplasia; achondroplasia; idiopathic short stature (ISS); GHD in adults; fractures in or of long bones, such as tibia, fibula, femur, humerus, radius, ulna, clavicula, matacarpea, matatarsea, and digit; fractures in or of spongious bones, such as the scull, base of hand, and base of food; patients after tendon or ligament surgery in e.g. hand, knee, or shoulder; patients having or going through distraction oteogenesis; patients after hip or discus replacement, meniscus repair, spinal fusions or prosthesis fixation, such as in the knee, hip, shoulder, elbow, wrist or jaw; patients into which osteosynthesis material, such as nails, screws and plates, have been fixed; patients with non-union or mal-union of fractures; patients after osteatomia, e.g. from tibia or 1st toe; patients after graft implantation; articular cartilage degeneration in knee caused by trauma or arthritis; osteoporosis in patients with Turner syndrome; osteoporosis in men; adult patients in chronic dialysis (APCD); malnutritional associated cardiovascular disease in APCD; reversal of cachexia in APCD; cancer in APCD; chronic abstractive pulmonal disease in APCD; HIV in APCD; elderly with APCD; chronic liver disease in APCD, fatigue syndrome in APCD; Chron's disease; impaired liver function; males with HIV infections; short bowel syndrome; central obesity; HIV-associated lipodystrophy syndrome (HALS); male infertility; patients after major elective surgery, alcohol/drug detoxification or neurological trauma; aging; frail elderly; osteo-arthritis; traumatically damaged cartilage; erectile dysfunction; fibromyalgia; memory disorders; depression; traumatic brain injury; subarachnoid haemorrhage; very low birth weight; metabolic syndrome; glucocorticoid myopathy; or short stature due to glucocorticoid treatment in children.

### Embodiment 99

A bile acid linker selected from the group of: A-W—B1-NH₂, A-W—B1-CHO, A-W—B1-LG, A-W—B1-C(O)NHCH₂—CH═CH₂ and

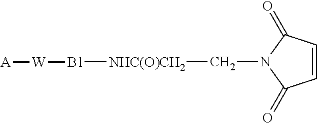

wherein A represents a bile acid residue, B1 represents a hydrophilic spacer, and W is a chemical group linking A and B1.

### Embodiment 100

The bile acid linker according to embodiment 99,

wherein A is defined as in one or more of embodiments 64-67,
wherein B1 is comprised by B as defined in one or more of embodiments 69-82 and/or
wherein W is defined as in embodiment 68.

### Embodiment 101

The bile acid linker according to embodiment 99, wherein the bile acid linker is selected from group consisting of: Linker 1, 2, 3, 4, 5, 6, 7, 8, 9, 10, 11, 12 and 13 as described herein.

### Chemistry

Chemistry 7361 chemistry I

In an aspect the present invention relates to preparation of a growth hormone conjugate of formula (I) wherein a GH compound is treated with a property-modifying group using TGase catalyzed chemistry. Initially, an aldehyde or a ketone functionality is installed by a two step reaction using amino alcohols that subsequently are treated with periodate to generate an aldehyde or keto functionality by oxidative cleavage. Non limited examples of amino alcohols for illustration only includes 1,3-diamino-2-propanol and 1-amino-2,3-dihydroxypropane.

In a further aspect the present invention relates to preparation of a growth hormone conjugate of formula (I) comprising treatment of an aldehyde or ketone derived from the GH compound with a property-modifying group-derived aniline or heteroaryl amine to yield an amine (III→IV).

In an embodiment, aldehyde derived from the GH compound is treated with property-modifying group-derived aniline or heteroarylamino.

The term “GH compound derived aldehyde (or ketone)” or “an aldehyde or ketone derived from the GH compound” is intended to indicate a GH compound to which an aldehyde or ketone functional group has been covalently attached, or a GH compound on which an aldehyde or ketone functional group has been generated. The preparation of GH compound-derived aldehydes, such as compound (III) illustrated below is well known to those skilled in the art, and any of these known procedures may be used to prepare the GH compound-derived aldehyde (III) required for the realization of the invention disclosed herein.

In one embodiment, the conjugate A-W—B-GH (IV) is prepared as illustrated below:

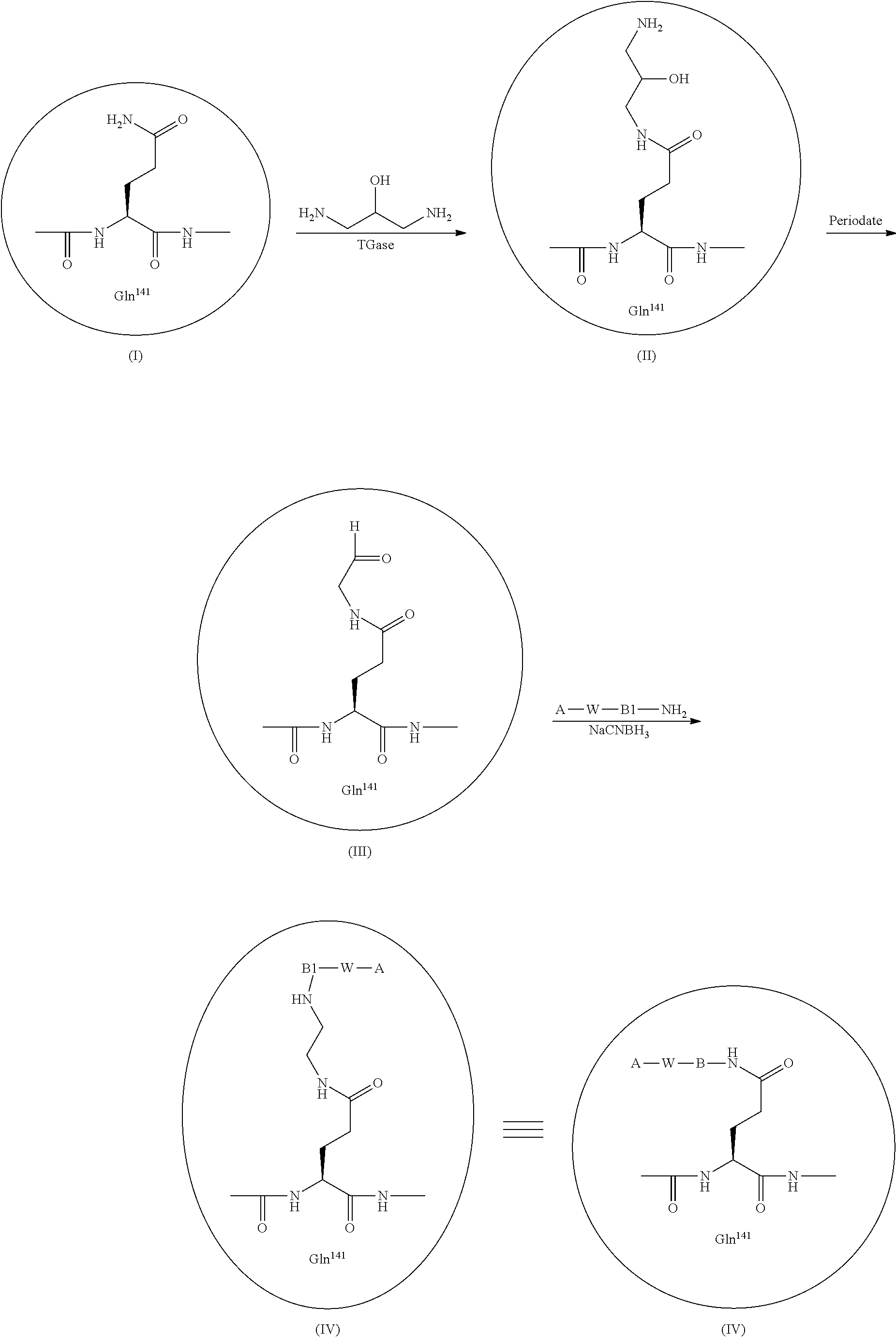

The TGase-mediated enzymatic reaction results in the modification of Gln at position 141 and/or 40 (II). The modified GH (II) is treated with periodate to cleave the amino-alcohol to provide a GH derived aldehyde (III). Conjugation of GH with A-W—B1-NH₂ occurs via reductive alkylation (III→IV). Reductive alkylation as exemplified herein is well-recognized to those skilled in the art.

Chemistry I-7361 chemistry I

In an aspect the present invention relates to preparation of a growth hormone conjugate of formula (I) wherein a GH compound is treated directly with a property-modifying group-derived amine, aniline or heteroaryl amine to yield a growth hormone conjugate (I→III) using TGase catalyzed chemistry.

In an embodiment, the GH compound is treated with a property-modifying group-derived amine, aniline or heteroaryl amine.

In one embodiment, the conjugate A-W—B-GH (III) is prepared as illustrated below:

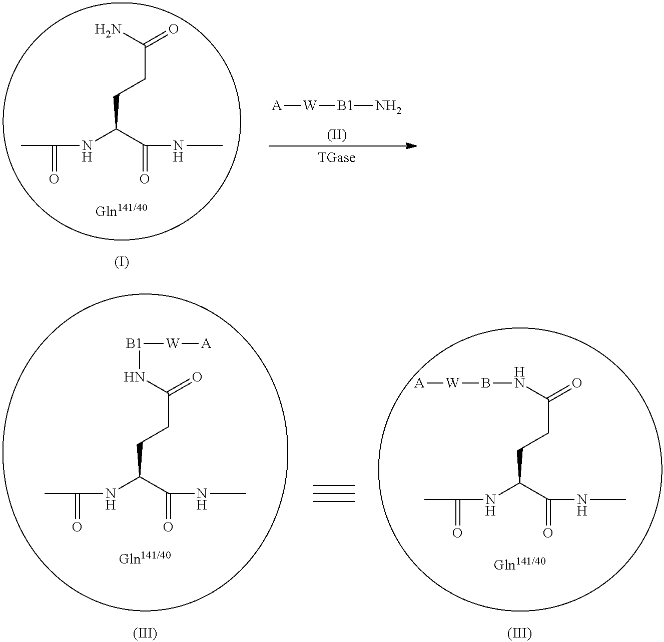

The TGase-mediated enzymatic reaction of GH (I) with A-W—B1-NH₂ (II) results in the modification of Gln at position 141 and/or 40 affording the conjugate A-W—B-GH (III).

Chemistry II—7361 chemistry I

In one embodiment, the GH conjugate A-W—B-GH (V) is prepared via conjugation to the N-terminal of GH as illustrated below:

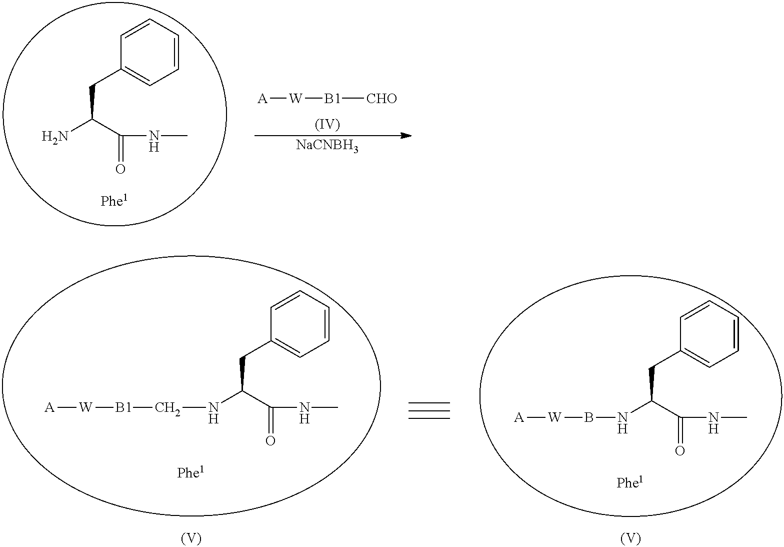

Conjugation of GH to A-W—B1-CHO (IV) occurs via reductive alkylation (GH→V). Reductive alkylation as exemplified above is well-recognized in the art for modifying the N-terminal of proteins e.g. GH

wherein the hydrophilic spacer B1 has the formula

—X₁—X₂—X₃—X₄—

wherein
X₁ is —W₁—[(CHR³)ₗ₁—W₂]ₘ₁-{[(CH₂)ₙ₁E1]ₘ₂—[(CHR⁴)ₗ₂—W₃]ₘ₃}ₙ₂-,
X₂ is —[(CHR⁵)ₗ₃—W₄]ₘ₄-{[(CH₂)ₙ₃E2]ₘ₅—[(CHR⁶)ₗ₄—W₅]ₘ₆}ₙ₄-,
X₃ is —[(CHR⁷)ₗ₅]ₘ₇-,
X₄ is a valence bond,
l1, l2, l3, l4, and l5 independently are selected from 0-10,
m1, m3, m4, m6 and m7 independently are selected from 0-6,
m2 and m5 independently are selected from 0-6,
n1, n2, n3 and n4 independently are selected from 0-6,
R³, R⁴, R⁵, R⁶ and R⁷ independently are selected from hydrogen, —C(O)OH, —C(O)NH₂, —S(O)OH, —S(O)₂OH, —NH—C(═NH)—NH₂, C₁₋₆-alkyl, aryl or hetaryl; wherein the alkyl, aryl and hetaryl groups optionally are substituted with halogen, —C(O)OH, —C(O)NH₂, —S(O)OH, —S(O)₂OH, —CN or —OH,
E1 and E2 independently are selected from —O—, —NR⁸—, —N(COR⁹)— or a valence bond; wherein R⁸ and R⁹ independently represent hydrogen or C₁₋₆-alkyl, W₁ to W₆ independently are selected from —NH—, —C(O)NH—, —NHC(O)—, —C(O)NHCH₂—, —CH₂NHC(O)—, —C(O)NHS(O)₂—, —S(O)₂NHC(O)—, —OC(O)NH—, —NHC(O)O—, —C(O)CH₂—, —CH₂C(O)—, —C(O)CH═CH—, —CH═CHC(O)—, —(CH₂)ₛ₂—, —C(O)—, —C(O)O—, —OC(O)—, or a valence bond; wherein s2 is 0 or 1.

### Chemistry IV

In one embodiment, the conjugate A-W—B-GH (IX) is prepared as illustrated below:

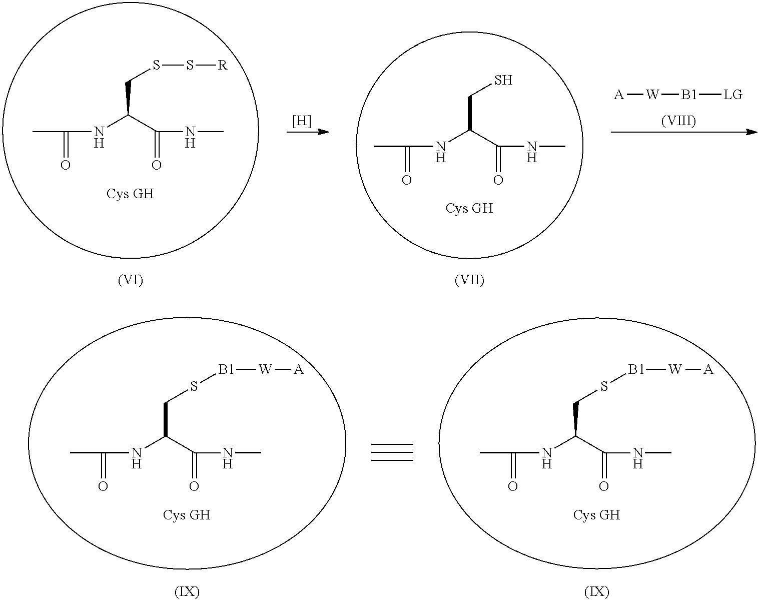

Wherein the GH compound contains a single cysteine residue being optionally protected as a mixed disulfide (GH-S—S—R¹⁰ (VI)) with R¹⁰ being a small organic moiety. Non limited examples of mixed disulfides may include disulfides between

cystamine (R¹⁰=—CH₂CH₂NH₂); cysteine (R¹⁰=—CH₂CH(C(O)OH)NH₂);

homocysteine (R¹⁰=—CH₂CH₂CH(C(O)OH)NH₂); and

gluthatione (R¹⁰=—CH₂CH(C(O)NH—CH₂C(O)OH)NH—C(O)CH₂CH₂CH(C(O)OH)NH₂).

The derivatization process utilise a linker A-W—B1-LG (VIII) wherein LG represent an inorganic leaving group such as —Cl, —Br, —I or an organic leaving group such as mesylate or tosylate. Conjugation of GH (VI) with A-W—B1-LG (VIII) occurs via nucleophilic substitution (VII→IX). Nucleophilic substitution as exemplified above is well-recognized in the art for modifying a free cysteine in a protein such as GH.

### Chemistry V

In one embodiment, the conjugate A-W—B-GH(XI) is prepared as illustrated below:

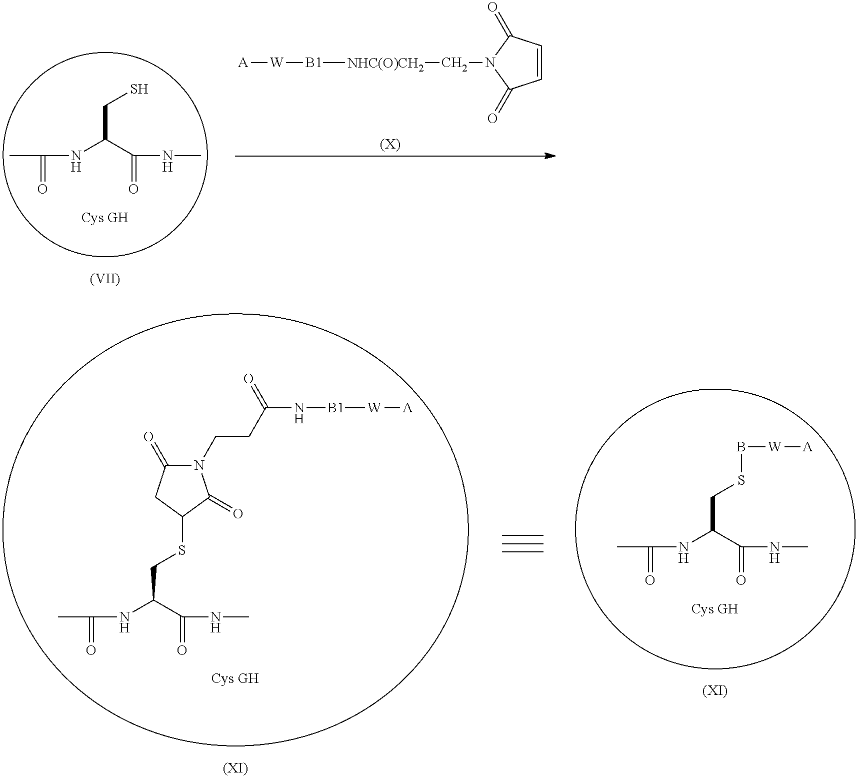

Deprotected Cys GH compound (VII) as obtained from (VI) above can be reacted with a malimide substituted linker (X) affording GH conjugate A-W—B1-NHC(O)CH₂CH₂-pyrrolidin-2,5-dione-3-GH(XI)

wherein the hydrophilic spacer B1 has the formula

—X₁—X₂—X₃—X₄—

wherein
X₁ is —W₁—[(CHR³)ₗ₁—W₂]ₘ₁-{[(CH₂)ₙ₁E1]ₘ₂—[(CHR⁴)ₗ₂—W₃]ₘ₃}ₙ₂-,
X₂ is —[*(CHR⁵)ₗ₃—W₄]ₘ₄-{[(CH₂)ₙ₃E2]ₘ₅—[(CHR⁶)ₗ₄—W₅]ₘ₆}ₙ₄-,
X₃ is —[(CHR⁷)ₗ₅]ₘ₇-,
X₄ is a valence bond,
l1, l2, l3, l4, and l5 independently are selected from 0-10,
m1, m3, m4, m6 and m7 independently are selected from 0-6,
m2 and m5 independently are selected from 0-6,
n1, n2, n3 and n4 independently are selected from 0-6,
R³, R⁴, R⁵, R⁶ and R⁷ independently are selected from hydrogen, —C(O)OH, —C(O)NH₂, —S(O)OH, —S(O)₂OH, —NH—C(═NH)—NH₂, C₁₋₆-alkyl, aryl or hetaryl; wherein the alkyl, aryl and hetaryl groups optionally are substituted with halogen, —C(O)OH, —C(O)NH₂, —S(O)OH, —S(O)₂OH, —CN or —OH,
E1 and E2 independently are selected from —O—, —NR⁸—, —N(COR⁹)— or a valence bond; wherein R⁸ and R⁹ independently represent hydrogen or C₁₋₆-alkyl,
W₁ to W₆ independently are selected from —NH—, —C(O)NH—, —NHC(O)—, —C(O)NHCH₂—, —CH₂NHC(O)—, —C(O)NHS(O)₂—, —S(O)₂NHC(O)—, —OC(O)NH—, —NHC(O)O—, —C(O)CH₂—, —CH₂C(O)—, —C(O)CH═CH—, —CH═CHC(O)—, —(CH₂)ₛ₂—, —C(O)—, —C(O)O—, —OC(O)—, or a valence bond; wherein s2 is 0 or 1.

### Chemistry VI

In one embodiment, the conjugate A-W—B-GH is prepared as illustrated below:

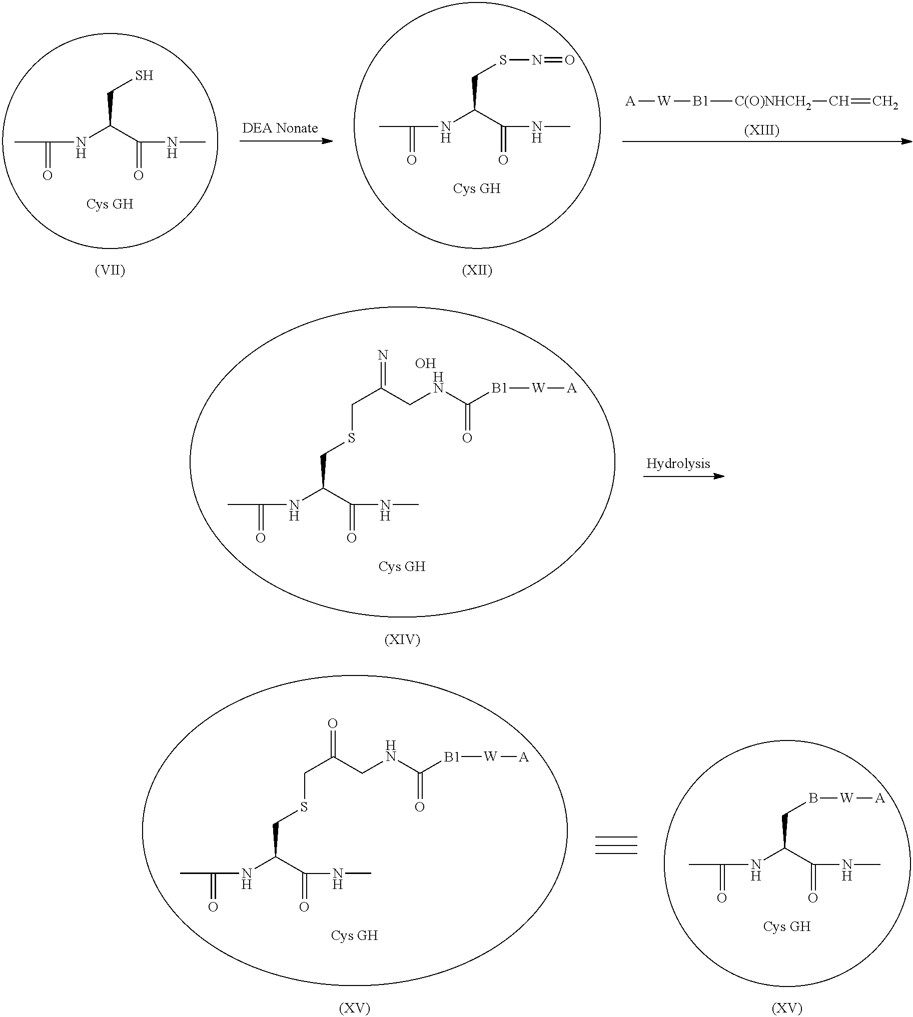

Bile acid linkers may be attached to single cys GH derivatives using S-nitrosyl chemistry as described in WO2009/024791.

Deprotected Cys GH compound (VII) is subjected to S-nitrosylation by addition of a NO donor such as DEA NOnate (Sigma Aldrich). Nitrosylated single cys GH (XII) is then reacted with an allyl amine substituted bile acid linker (XIII) affording oxime (XIV) which after hydrolysis affords GH conjugate A-W—B1-C(O)NHCH₂C(O)CH₂-Cys GH (XV)

wherein the hydrophilic spacer B1 has the formula

—X₁—X₂—X₃—X₄—

wherein
X₁ is —W₁—[(CHR³)ₗ₁—W₂]ₘ₁-{[(CH₂)ₙ₁E1]ₘ₂—[(CHR⁴)ₗ₂—W₃]ₘ₃}ₙ₂-,
X₂ is —[(CHR⁵)ₗ₃—W₄]ₘ₄-{[(CH₂)ₙ₃E2]ₘ₅—[(CHR⁶)ₗ₄—W₅]ₘ₆}ₙ₄-,
X₃ is —[(CHR⁷)ₗ₅]ₘ₇-,
X₄ is a valence bond,
l1, l2, l3, l4, and l5 independently are selected from 0-10,
m1, m3, m4, m6 and m7 independently are selected from 0-6,
m2 and m5 independently are selected from 0-6,
n1, n2, n3 and n4 independently are selected from 0-6,
R³, R⁴, R⁵, R⁶ and R⁷ independently are selected from hydrogen, —C(O)OH, —C(O)NH₂, —S(O)OH, —S(O)₂OH, —NH—C(═NH)—NH₂, C₁₋₆-alkyl, aryl or hetaryl;
   wherein
the alkyl, aryl and hetaryl groups optionally are substituted with halogen,
—C(O)OH, —C(O)NH₂, —S(O)OH, —S(O)₂OH, —CN or —OH,
E1 and E2 independently are selected from —O—, —N(R⁸)—, —N(C(O)R⁹)— or a valence bond; wherein R⁸ and R⁹ independently represent hydrogen or C₁₋₆-alkyl, W₁ to W₆ independently are selected from —NH—, —C(O)NH—, —NHC(O)—, —C(O)NHCH₂—, —CH₂NHC(O)—, —C(O)NHS(O)₂—, —S(O)₂NHC(O)—, —OC(O)NH—, —NHC(O)O—, —C(O)CH₂—, —CH₂C(O)—, —C(O)CH═CH—, —CH═CHC(O)—, —(CH₂)ₛ₂—, —C(O)—, —C(O)O—, —OC(O)—, or a valence bond; wherein s2 is 0 or 1.

### EXAMPLES

The invention will be further defined by reference to the following examples, which describe the preparation and characterization of the various compounds described herein and methods for assaying their biological activity. It will be apparent to those skilled in the art that many modifications, both to the materials and methods may be practiced without departing from the scope of the invention.

Abbreviations:

amu=atomic mass units

CV=column volumes

hr(s)=hour(s)

Hz=hertz

L=liter(s)

M=molar

mbar=millibar

mg=milligram(s)

min.=minute(s)

mL=milliliter(s)

mM=millimolar

mm=milimeter(s)

mmol=millimole(s)

nmol=nanomole(s)

mol=mole(s)

μL=microliters

N=normal

nm=nanometer(s)

sec=second(s)

ppm=parts per million

ESI=electrospray ionization

i.v.=intravenous

m/z=mass to charge ratio

MS=mass spectrometry

HPLC=high pressure liquid chromatography

RP=reverse phase

HPLC-MS=high pressure liquid chromatography—mass spectrometry

NMR=nuclear magnetic resonance spectroscopy

p.o.=per oral

rt or RT=room temperature

s.c.=subcutaneous

Rₜ=retention time

Boc=tert butyloxycarbonyl

O-t-Bu=tert butyl ester

t-Bu=tert butyl

Boc-4-ABZ—OH=4-tert-Butoxycarbonylamino-benzoic acid

DCM=dichloromethane, CH₂Cl₂, methylenechloride

DIC=diisopropylcarbdiimide

DIPEA=N,N-diisopropylethylamine

DMF=N,N-dimethylformamide

DMSO=dimethylsulfoxide

DTT=dithiothreitol

EDAC=1-ethyl-3-(3-dimethylaminopropyl)carbodiimide hydrochloride

Et₂O=diethyl ether

EtOAc=ethyl acetate

Fmoc=9H-fluoren-9-ylmethoxycarbonyl

Fmoc-Glu-O-t-Bu=N-Fmoc-glutamic acid-1-t-butyl ester

Fmoc-Lys(Mtt)-OH═(S)-6-[(Diphenyl-p-tolylmethyl)amino]-2-(9H-fluoren-9-yl-methoxycarbonylamino)hexanoic acid

Fmoc-OEG-OH=(2[2-(Fmoc-amino)ethoxy]ethoxy)acetic acid

OEG=(2[2-(amino)ethoxy]ethoxy)acetyl

Fmoc-Thx-OH═N-Fmoc-trans-4-aminomethylcyclohexancarboxylic acid

H₂O=water

HOBt=1-hydroxybenzotriazole

MeCN=acetonitrile

MeOH=methanol

MSNT=1-(Mesitylene-2-sulfonyl)-3-nitro-1,2,4-triazole

MTP=3-methyl-thio-1-propanol

NaCl=sodium chloride

NaOH=sodium hydroxide

NMP=N-methylpyrrolidin-2-one

OEG=(2[2-(amino)ethoxy]ethoxy)acetic acid

TFA=trifluoroacetic acid

THF=tetrahydrofuran

TIS=triisopropylsilane

TSPP: Tris(3-sulfophenyl)phosphine trisodium salt

CDCl₃=deuterio chloroform

CD₃OD=tetradeuterio methanol

DMSO-d₆=hexadeuterio dimethylsulfoxide

TNBS=trinitrobenzensulfonic acid

TSTU=O—(N-Succinimidyl)-1,1,3,3-tetramethyl uranium tetrafluoroborate

TFAA=Trifluoroacetic Acid Anhydride

TEA=Triethanolamine

### Method 1. General Method for Preparing a hGH Compounds.

The gene coding for the growth hormone compound was inserted recombinant into a plasmid vector. Mutations were introduced by using QuikChange site-directed mutagenesis kit (Stratagene). A suitable E. coli strain was subsequently transformed using the plasmid vector. Protein was expressed as soluble protein with an N-terminal Histidine rich peptide tag suitable for immobilised metal affinity chromatography purification. hGH or GH variants may be expressed with an N-terminal methionine or as a MEAE fusion from which the MEAE sequence is subsequently cleaved off.

Cell stock was prepared in 50% glycerol and stored at −80° C. Glycerol stock strain was inoculated into LBA plates and subsequently incubated at 37° C. overnight. The content of each plate was washed with LB medium and diluted into 500 mL LB+AMP medium for expression. The cultures were incubated at 37° C. with shaking at 220 rpm until OD₆₀₀ 0.6 was reached. Succeeding induction was performed using 0.2 mM IPTG at 30° C. for 6 hrs, giving a final OD₆₀₀ of 2.0. Cells were finally harvested by centrifugation.

Cells were subsequently suspended in 20 mM Tris-HCl, pH 8.5 and disrupted using a cell disrupter at 30 kPSI. The supernatant was collected by centrifugation and subsequently subjected to chromatographic purification.

The purification was performed using immobilized metal affinity chromatography as capturing step, followed by removal of the peptide tag using di-amino-peptidase from Unizyme. Final purification was achieved by ion-exchange chromatography. The purification could also be achieved by using but not limited to ion-exchange chromatography, hydrophobic interaction chromatography, affinity chromatography, size exclusion chromatography and membrane based separation techniques known to a person skilled in the art.

### Method 2. Isolation and Purification of Cys Mutated GH Protein:

The method is exemplified using MEAE-hGH[Q84C, Y143C, L101C]. Alternative mutants may be isolated and purified in a similar way possible with minor modification to the isolation and purification scheme based on common general knowledge in the art.

Cells are harvested by centrifugation (6056×G for 30 min) and may be stored at −80° C. Homogenization: Cell pellets (2580 g) were dissolved in ca. 20 kg buffer (20 mM Tris, 0.1% Tween-20, 5 mM EDTA, pH=8.5) and pH adjusted to 8.5 followed by homogenization with two pressures drops 1000 and 200 bar. The cell homogenate (˜20 kg) was diluted 1:1 in 8M urea, resulting in a 4M urea concentration. To this solution was added cystamine 2HCl (18 g) (0.9 g/L) and the resulting mixture incubated over night at 5° C. with gentle stirring. Filtration: The homogenate was filtered by dead end filtration 1.0 and 0.5 μm resulting in approx. 42 kg permeate. Purification of MEAE-hGH[Q84C, Y143C, L101C] consists of three chromatographic steps and an enzyme digestion to remove the N-terminal MEAE-sequence. MEAE-hGH[Q84C, Y143C, L101C] was captured using Q Sepharose XL (GE Healthcare) and eluted by linear gradient elution in a Tris-NaCl buffer, pH 8.5. To the collected pool was added 1.0 M NaCl before loading onto Phenyl Sepharose 6FF (high sub) (GE Healthcare). Step elution in WFI was used to elute the protein and lower the conductivity for enzyme digestion.

The MEAE-sequence was removed using the enzyme DAPI (dipeptidylaminopeptidase 1). DAPI cleaves the two N-terminal amino acids off at a time and stops after AE as the second amino acid in hGH is a proline residue, providing a natural stop for DAPI digestion. Digestion was carried out at 40° C. at a protein concentration of 2 mg/mL, pH 4.3. The reaction was followed by LC-MS and stopped after approx. 60 min when digestion was complete. After cooling to 5° C., 39% v/v cold ethanol (99%) was added to iso-precipitate the protein and pH was adjusted to 4.9. The reaction mixture was stored at 5° C. for at least 2 hrs. to precipitate the protein.

Precipitated hGH[Q84C, Y143C, L101C] was re-dissolved in a 7 M urea-triethanolamine buffer, pH 7.5, and purified on Source 30Q (GE Healthcare) to separate target protein from minor amounts of un-cleaved material and other impurities. Final product is eluted by linear gradient elution in a triethanolamine buffer, pH 7.5. Product purity is >97% by RP-HPLC analysis (UV 214 nm).

### Method 3. Methods for Preparing Cholic Acid Conjugated Compounds.

### 1. Coupling of Bile Acid Linker of General Formula A-W—B1-CHO to the N-Terminal Phenylalanine of GH Compound (I):

The bile acid linker of interest is typically synthesized as dialkyl acetal (i.e. A-W—B1-CH(OR)₂), and need to be converted into free aldehyde before conjugation. This can be done as follows: The bile acid linker dialkyl acetal is treated with acid (e.g. TFA) and evaporated to dryness. The free aldehyde obtained is used without further purification.

A solution of GH in buffer (f.ex. HEPES, pH 7.0) is then added portion wise while maintaining pH at 7.0 by addition of 1N NaOH. NaCNBH₃ dissolved in water is then added portions wise with intervals of 5 min. Typically, a clear solution is obtained after 1 hr. of stirring. The reaction flask is wrapped in tin-foil and the reaction mixture gently stirred at room temperature over night. A reaction sample is analyzed by LC/MS to verify product formation.

The hGH conjugate is then purified using a combination of IEX and HIC chromatography, size exclusion column and ultra-filtration. Usually conjugates are obtained having 95% purity or above.

### 2. Coupling of Bile Acid Linker of General Formula A-W—B1-NH2 to Transaminated and Oxidised GH Compound (I)

The use of a transglutaminase to attach an aldehyde handle to GH on glutamine residues has previously been described in WO2005/070468. The method may be used in accordance with the present invention for attachment of a bile acid based linker of formula A-W—B1—NH2. The TGase used is microbial transglutaminase from Streptoverticillium mobaraense according to U.S. Pat. No. 5,156,956.

The reaction may be performed as follows: GH (I) is initially transaminated with 1,3-diamino-2-propanol (II) as described in WO2005/070468:

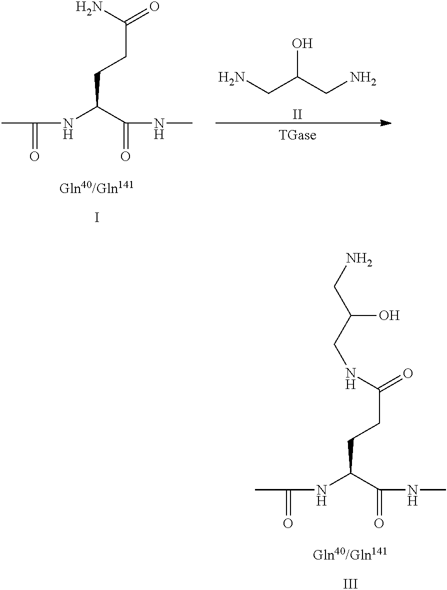

In next step, transaminated GH (III) is added periodate. The oxidation is typically done at low temperature, such as 4-10° C. over 30 min. optionally in the dark. Periodate may oxidize metheonine residues in GH to their corresponding metheonine sulfoxide residues. To minimize this oxidation risk, small molecule organic thioethers may be added during periodate oxidation. A suitable organic thioether is 3-methylthiopropane-1-ol but the skilled person will be able to suggest others.

Oxidation of transaminated GH compound (III):

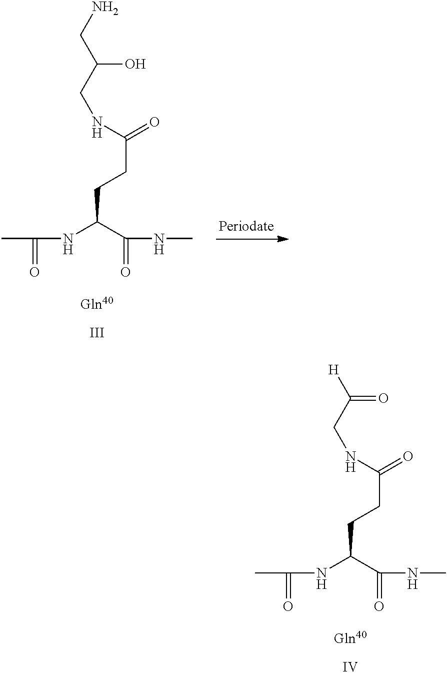 [SMILES: CNC(=O)[C@H](CCC(=O)NCC(O)CN)NC(C)=O ; "III" ; CNC(=O)[C@H](CCC(=O)NCC=O)NC(C)=O ; "IV"]

Buffer change may be performed in order to obtain an acid solution required for efficient sodium cyano borohydride reduction. Typically, an excess of A-W—B1-NH2 amine is used, and sodium cyanoborohydride may be added in smaller portions over time.

Reductive amination of (IV) with bile acid linker (V):

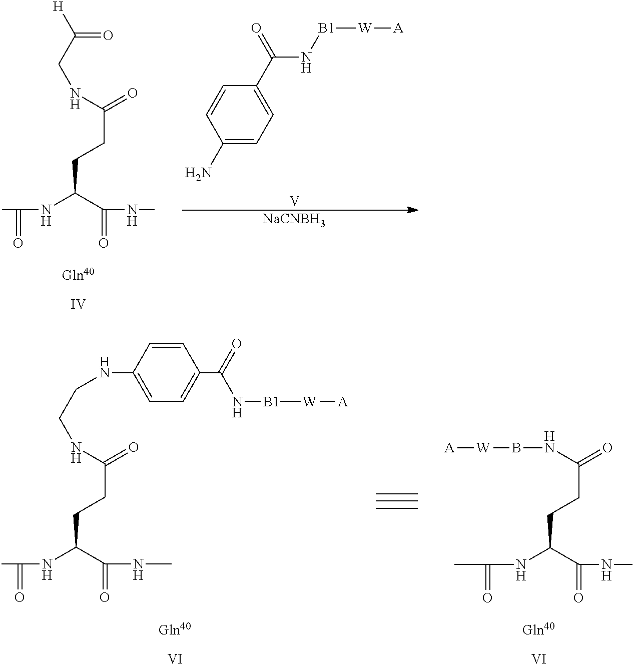

The later reaction may be performed as follows:

A solution of oxidized transaminated GH (IV) is added a solution of bile acid linker (V) in a mixture of AcOH (1.5 mL) and 50 mM MES (0.5 mL) at pH 6.00. The resulting reaction mixture is gently shaken at RT for 30 min. at which time a NaCNBH₃ solution (15 μL, (22 mg NaCNBH₃ dissolved in 500 μL Milli-Q water+AcOH (15 μL))) is added. The sample is covered with tin foil and stirrer over night at RT.

The conjugate can be isolated by anion exchange chromatography as follows: Acetic acid is removed by buffer changed with pure water (3×) using Amicon Ultra15 devices (Ultracel 10K tubes) by centrifugation at 4000 rpm/min. for 3×8 min. The mixture is then buffer changed to 20 mM TEA, pH: 8.50 using Amicon Filter devises and diluted to a final volume of 50 mL with 20 mM TEA, before loading it on a HiLoad Q Sepharose, 26/10 column. The column is initially washed with 20 mM TEA, pH 8.50 (buffer A) and then eluted with 20 mM TEA, 500 mM NaCl, pH 8.50 (buffer B) using a 0-100% (B) gradient over 20 CV, with a flow rate of 2 mL/min. The pooled fractions were buffer changed 5 times to 10 mM ammoniumbicarbonate buffer in pure water using Amicon Ultra15 devices (Ultracel 10K tubes) by centrifugation at 4000 rpm/min. for 3×8 min.

### 3. Coupling of Bile Acid Linker of General Formula A-W—B1-LG (VIII) to GH Compound (VII) Having an Internal Free Single Cys.

1) Liberation of free Cys GH (VII) via reduction of disulfide (VI) with a suitable selective reducing agent [H]:
2) Alkylation of free Cys GH (VII) with a leaving group (LG) activated bile acid linker (VIII) affording Cys conjugated GH compound (IX)

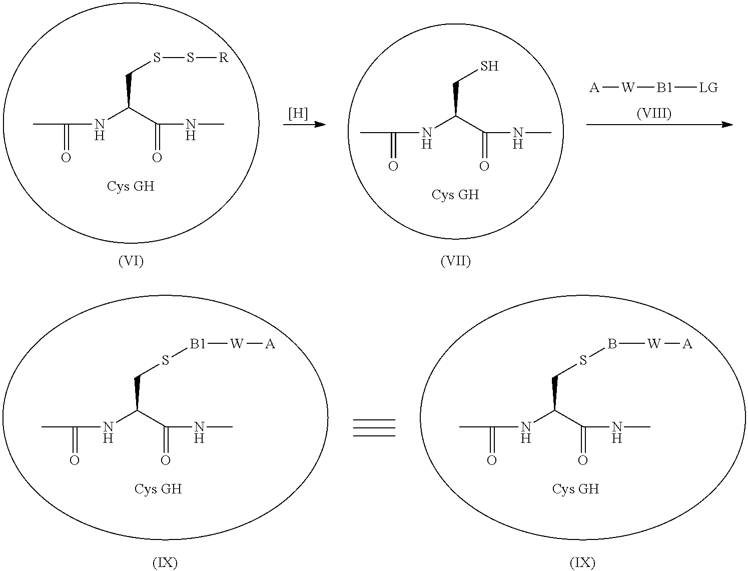

The cysteine residue is optionally protected as a mixed disulfide (GH-S—S—R (VI)) with R being a small organic moiety. Non limited examples of mixed disulfides may include disulfides between cystamine (R=—CH₂CH₂NH₂); cysteine (R=—CH₂CH(C(O)OH)NH₂); homocysteine (R=—CH₂CH₂CH(C(O)OH)NH₂); and gluthatione (R=—CH₂CH(C(O)NH—CH₂C(O)OH)NH—C(O)CH₂CH₂CH(C(O)OH)NH₂).

The derivatization process utilise a bile acid linker A-W—B1-LG wherein LG represent an inorganic leaving group such as —Cl, —Br, —I or an organic leaving group such as mesylate or tosylate. Conjugation of GH with A-W—B1-LG occurs via nucleophilic substitution (VII—IX). If the derivatization process employees maleimides of acryl functionalities as reactive group, conjugation occur by sulfide addition to the double bond of the maleimide or the acrylate functionality.

GH conjugates prepared as described in the invention may be purified by standard methods sych as HPLC, size exclusion chromatography and by ion-exchange chromatography.

It should be noted, that the general coupling methods described above merely serves for illustration purposes and a person skilled in the art may chose other buffer solutions, purification methods, and reaction condition for preparing the conjugates.

### Method 4. Protein Chemical Characterization of Purified Growth Hormone Compounds.

### Maldi-T of Mass Spectrometry

Molecular weights were determined using the Autoflex Maldi-T of instrument (Bruker). Samples were prepared using alfa-cyano-4-hydroxy-cinnamic acid as matrix.

The intact purified protein was analysed using MALDI-MS. The observed mass corresponded to the theoretical mass deduced from the amino acid sequence.

The linkage of the disulfide bonds in each compound may be demonstrated by peptide mapping using trypsin and AspN digestion followed by MALDI-MS analysis of the digest before and after reduction of the disulfide bonds with DTT.

### Capillary Electrophoresis

Capillary electrophoresis was carried out using an Agilent Technologies 3DCE system (Agilent Technologies). Data acquisition and signal processing were performed using Agilent Technologies 3DCE ChemStation. The capillary was a 64.5 cm (56.0 cm efficient length) 50 μm i.d. “Extended Light Path Capillary” from Agilent. UV detection was performed at 200 nm (16 nm Bw, Reference 380 nm and 50 nm Bw). The running electrolyte was phosphate buffer 50 mM at pH 7 (method A). The capillary was conditioned with 0.1M NaOH for 3 min, then with Milli-Q water for 2 min and with the electrolyte for 3 min. After each run, the capillary was flushed with milli-Q water for 2 min, then with phosphoric acid for 2 min, and with Milli-Q water for 2 min. The hydrodynamic injection was done at 50 mbar for 4.0 sec. The voltage was +25 kV. The capillary temperature was 30° C. and the runtime was 10.5 min.

### RP-HPLC

RP-HPLC analysis was performed on an Agilent 1100 system using a Vydac 218TP54 4.6 mm×250 mm 5 μm C-18 silica column (The Separations Group, Hesperia). Detection was by UV at 214 nm, 254 nm, 280 nm and 301 nm. The column was equilibrated with 0.1% TFA/H₂O and the sample was eluted by a suitable gradient of 0 to 90% MeCN against 0.1% TFA/H₂O.

### LC-MS

LC-MS analysis was performed on a PE-Sciex API 100 or 150 mass spectrometer equipped with two Perkin Elmer Series 200 Micro-pumps, a Perkin Elmer Series 200 auto-sampler, a Applied Biosystems 785A UV detector and a Sedex 75 Evaporative Light scattering detector. A Waters Xterra 3.0 mm×50 mm 5μ C-18 silica column was eluted at 1.5 mL/min at RT. It was equilibrated with 5% MeCN/0.1% TFA/H₂O and eluted for 1.0 min with 5% MeCN/0.1% TFA/H₂O and then with a linear gradient to 90% MeCN/0.1% TFA/H₂O over 7 min. Detection was by UV detection at 214 nm and Evaporative light Scattering. A fraction of the column eluate was introduced into the ionspray interface of a PE-Sciex API 100 mass spectrometer. The mass range 300-2000 amu was scanned every 2 seconds during the run.

### Quantification of Protein

Protein concentrations were estimated by measuring absorbance at 280 nm using a NanoDrop ND-1000 UV-spectrophotometer.

### Enzymatic Peptide Mapping for Determination of Site(s) of Derivatization

Peptide mapping was performed using Asp-N digestion of the reduced and alkylated protein. First the protein was treated with DTT and iodoacetamide according to standard procedures. The alkylated product was purified using HPLC. Subsequently the alkylated purified product was digested overnight with endoprotease Asp-N (Boehringer) at an enzyme:substrate ratio of 1:100. The digest was HPLC separated using a C-18 column and standard TFA/MeCN buffer system. The resulting peptide map was compared to that of un-derivatized hGH and fractions with different retention times were collected and further analyzed using Maldi-tof mass spectrometry.

### SDS Page

SDS poly-acrylamide gel electrophoresis was performed using NuPAGE 4%-12% Bis-Tris gels (Invitrogen NP0321BOX). The gels were silver stained (Invitrogen LC6100) or Coomassie stained (Invitrogen LC6065).

### Protein Chromatography

Protein chromatography was performed on an Äkta Explorer chromatographic system and columns from GE Health Care. Anion exchange was done using a Q-Sepharose HP 26/10 column. Starting buffer was 20 mM triethanolamine buffer pH 8.5 and eluting buffer was starting buffer+0.2 M NaCl. The compounds were typically eluted with a gradient of 0-75% eluting buffer over 15 column volumes. De-salting and buffer exchange was performed using a HiPrep 26/10 column.

TNBS test

A solution of 10% DIPEA in DMF (solution 1) and a solution of 1 M aqueous TNBS (solution 2) was prepared. A few resin beads were placed in a small test tube and 1-3 drops of each solution (1 and 2) were added. After a short mixing time the mixture was left at RT for 10 min. followed by inspection of the beads. Intensely orange or red beads indicate positive results (i.e presence of free amines); yellow or slightly orange beads indicate slightly positive and colorless beads are negative.

### Method 5. Analysis of the Biological Activity of the Purified Growth Hormone Compounds.

The biological activity of hGH compounds are measured in a cell based receptor potency proliferation assay, namely a BAF assay. The method is general for the hGH compounds.

The BAF-3 cells (a murine pro-B lymphoid cell line derived from the bone marrow) is IL-3 dependent for growth and survival. IL-3 activates JAK-2 and STAT which are the same mediators GH is activating upon stimulation.

The BAF-3 cells are transfected with a plasmid containing the hGH receptor. Clones able to proliferate upon stimulation with hGH are turned into hGH-dependent cell lines hereafter referred to as BAF3-GHR. The cell lines respond to GH with a dose-related growth pattern and can therefore be used to evaluate the effect of different hGH compounds in a proliferation assay.

The BAF-3 GHR cells are grown in starvation medium (culture medium without GH) for 24 hrs. at 37° C., 5% CO₂. The cells are centrifuged, the medium is removed and the cells are re-suspended in starvation medium to 2.22×10⁵ cells/ml. Portions of 90 μL of the cell supernatant are seeded into microtiter plates (96 well NUNC-clone). Different concentrations of growth hormone compound are added to the cells, and the plates are incubated for 72 hrs. at 37° C., 5% CO₂.

AlamarBlue is a redox indicator, AlamarBlue® (BioSource cat no Dal 1025) which is reduced by reactions innate to cellular metabolism and, therefore, provides an indirect measure of viable cell number. The AlamarBlue® is diluted 6 times (5 μL AlamarBlue®+25 μL starvation medium) and 30 μL of the diluted AlamarBlue® is added to each well. The cells are then incubated for another 4 hrs. Finally the metabolic activity of the cells is measure in a fluorescence plate reader using an excitation filter of 544 nM and an emission filter of 590 nM.

The result for a given compound is expressed as the ratio between EC₅₀ of said compound and the EC₅₀ of wthGH run in parallel.

### Method 6. In Vivo Dose-Response Study in Hypophysectomised Sprague Dawley Rats Previous 3A

The in vivo dose-response relationship may be studied in hypophysectomised male Sprague Dawley rats. The hypophysectomised rat is a well known and recognised animal model of growth hormone deficiency, where no production of growth hormone occurs after the surgical removal of the pituitary gland. This also leads to low circulating levels of insulin-like growth factor-1 (IGF-1) another important clinical feature of growth hormone deficiency in humans.

The hypophysectomy are performed on 4 week old male rats weighing 90-100 g. The animals entered the study 3-4 weeks after the surgery weighing 100-110 g. Animals with a body weight gain of more than 10% during the 3-4 weeks after surgery were not allowed to enter the study.

Usually seventy hypophysectomised Sprague Dawley rats are randomly allocated to seven dosing groups with ten animals in each group. One group receives vehicle only and served as an untreated control group. Three groups receives test compound (such as a hGH variant) 33, 3.3 and 0.33 nmol respectively and three groups received hGH as a comparator 50, 5.0 and 0.5 nmol respectively. Both compounds and vehicle were administered as a single subcutaneous dose in the neck. The body weight are measured daily between 8-10 am for one week.

Evaluation of a dose-dependent increase in body weight may be obtained by compareing body weight on Day 0 with that of Day 7.

A sigmoidal dose-response equation is fitted to the experimental data (increase in body weight between Day 0-7) by non-linear regression analysis in order to calculate parameter estimates of Eₘₐₓ, E₀ and ED₅₀. The equation has a sigmoidal dose-response built-in equation in GraphPad Prism version 4.00 for Windows (GraphPad Software Inc., San Diego, USA). Data including parameter estimates and 95% confidence intervals are usually presented.

No difference in parameter estimates of E₀ and Eₘₐₓ was observed for hGH[Q84C, Y143C] and hGH. However ED₅₀ was significantly lower for hGH[Q84C, Y143C] compared to hGH indicating an increased in vivo potency of hGH[Q84C, Y143C].

### Method 7. Receptor Interaction Studies by Surface Plasmon Resonance Analysis.

Receptor interaction of hGH compounds may be analyzed using surface plasmon resonance analysis. The method is general for the hGH compounds. The interaction of hGH and analogues with hGH binding protein (hGHBP) are studied by surface plasmon resonance using a Biacore T100 instrument (GE Healtcare, Sweden). Anti-hGH mAb (Fitzgerald Industries International, USA, #10G05B) is immobilized onto a CM-5 chip according to manufacturers instruction at a level of typically 5000 RU. wthGH or analogues were captured at 10-25 μg/mL in running buffer (10 mM HEPES, 0.15 M NaCl, 30 mM EDTA, 0.05% Surfactant P20, pH 7.4), which resulted in 250-400 RU captured ligand. hGHBP at a concentration of 0-800 nM is subsequently injected over the surface at 30 μL/min. A surface with immobilized anti-hGH mAb but without captured hGH is used as reference.

Kinetic data is analyzed with Biacore™ Evaluation Software 2.0 with the 1:1 Langmuir binding model.

### Method 8. Assay for Measuring Rate of Protease Degradation of Wild Type hGH and GH Compounds

The compound of interest is digested by a relevant protease (Trypsin, Chymotrypsin, Pepsin, Elastase, Factor VIIa, Factor Xa, Proteinase K, Carboxy peptidase, DPPIV, Neutral Endopeptidase, Granzyme B, Proline-endopeptidase, Staphylococcal peptidase I, Thermolysin, Thrombin, Arg-C proteinase, Asp-N endopeptidase, Caspase 1-10, Clostripain, Enterokinase, Glutamyl endopeptidase, Granzyme B, LysC, LysN, Proline-endopeptidase and Staphylococcal peptidase I or tissue extracts.) in an appropriate buffer (e.g. PBS or ammonium bicarbonate) at 37° C. for up till 24 hrs. Proteolytic degradation is assessed by a HPLC assay.

### Proteolytic Digestion:

100 μL of test compound solution at 1 mg/mL in ammonium bicarbonate buffer is degraded by enzyme for up till 24 hrs. at 37° C. Sub-samples are taken to various time points and the proteolytic reaction is stopped by acidifying the sample by 10 times dilution into 1% TFA. These diluted samples are analysed by reversed phase HPLC to estimate the degree of proteolytic digestion.

### HPLC Method:

10 μL of the above solution is injected on a reversed phase Vydac C4 2×150 mm column eluted with a linear gradient from 0.1% TFA in water to 100% MeCN containing 0.1% TFA over a period of 30 min. at a flow rate of 0.2 mL/min. Detection of peaks is performed at 214 nm UV absorption. % intact compound at time point t=T is calculated from the peak area at time point t=T (A_{T}) and the peak area at t=0 (A₀) as (A_{T}/A₀)×100%.

The results provided in the examples below were obtained after 4 hrs. (T=4 in above equation). % intact compound is plotted against time using GraphPad Prims software ver. 5.01. T_{1/2} is calculated as one phase decay also by GraphPad Prism software. Enzymes used in the example are elastase (Sigma from porcine pancrease) and chymotrypsin (Roche sequencing grade). Buffer is 50 mM ammonium bicarbonate pH 8.5.

Method 9. Pharmacokinetic Properties of Growth Hormone Compounds by Intra-Intestinal and i.v. Administration to Gottingen Minipigs

Usually eight Göttingen male minipigs obtained from Ellegaard Göttingen Minipigs NS weighing approximately 30 kg are used at the study. After a two week acclimatisation period the animals are subjected to surgery, where two central venous catheters are inserted in each animal. After surgery the animals are housed in their normal individual pens. The animals are weighed prior to dosing. For animals that receive intra-intestinal dosing, straw and bedding material are removed from the pens 2 days prior to dosing in order to ensure absence of foreign material in the GI tract.

For i.v. dosing 20 nmol/kg is given through the central short catheter, which is flushed with 2 mL saline post administration.

For intra-intestinal dosing 800 nmol/kg is administered by an endoscope (Pentax gastroscope). The gastroscope is inserted orally and passed through the oesophagus and ventricle through the pylorus to the jejunum. The test formulation is released in the jejunum and the gastroscope is withdrawn. The minipigs are sedated with Sedator and Propofol during the dosing procedure, and Antisedan is given intramuscularly after the procedure.

Bloods samples are usually collected at the following time points: i.v. dosing: Predose, 5 min., 15 min., 30 min., 45 min., 1, 1.5, 2, 3, 4, 6, 8, 24, 48 and 72 hrs. after dosing.

Intra-intestinal dosing: Predose, 15 min., 30 min., 45 min., 1, 1.5, 2, 3, 4, 6, 8, 24, 48 and 72 hrs. after dosing.

At each sampling time 2 mL of blood is drawn from each animal. The blood samples are taken through the central catheter. After each blood sample the catheter is rinsed with 5 mL heparin (10 U/mL) in saline. During sampling the first ml blood is discarded. Aseptic techniques are required during the blood sampling in order to avoid bacterial contamination and the risk of clotting.

Blood samples are collected in test tubes containing EDTA 8 mM. Blood samples are kept on ice for max. 20 min. before centrifugation (4000 rpm, 4° C., 10 min.). Plasma is collected immediately and stored at −20° C. until assayed. Plasma samples are analysed for test compounds.

Test compound concentrations are determined by a sandwich ELISA using a guinea pig anti-hGH polyclonal antibody as catcher, and biotinylated hGH binding-protein (soluble part of human GH receptor) as detector. The limit of detection of the assay is 0.2 nM.

For i.v. administration the compounds are dissolved to 0.6 mM in Glycine 20 mg/ml, Mannitol 2 mg/mL, NaHCO₃ 2.4 mg/mL, pH adjusted to 8.2.

For intra-intestinal administration the compounds are dissolved to 6 mM in Glycine 20 mg/mL, Mannitol 2 mg/mL, NaHCO₃ 2.4 mg/mL, pH adjusted to 8.2.

### Method 10. Pharmacokinetic Properties of Growth Hormone Compounds by Intra-Intestinal Administration to Sprague Dawley Rats

Usually 24 Sprague Dawley rats, approx. 250-300 g are obtained from Taconic M&B (Rekv. No. 2010 0824). The animals are housed in type IV cages with 4-5 animals in each cage. The rats have free access to drinking water (tap water) during the study, but are fasted for 18 hrs. prior to dosing. During the fasting the animals are placed in cages on grid without bedding or nesting material and the food is removed at 2 pm on the day before dosing in order to ensure a fasting period of at least 18 hrs. During the fasting period the animals have free access to drinking water.

At the day of the operation the rats are weighted prior to the anaesthesia in order to make accurate dose calculations of the analgesics. Immediately after weighing, the animals receive a pre-analgesic dose of Rimadyl Vet. 50 mg/mL diluted in Saline (1+9), 0.1 mL/100 g s.c.

Although the study is not a sterile study the operative procedures are carried out as aseptic as possible. The operation area is disinfected with 70% ethanol and precautions are made to minimize the contamination of the surgical area.

The rats are anaesthetised with Isofluran 3% in the initial phase until the animals are at sleep, then the Isofluran is reduced to 2-2.5% during the rest of the surgery. The abdomen is shaved and disinfected with 70% ethanol before surgery.

Immediately before surgery each rat receives an s.c. dose of Temgesic 0.3 mg/mL diluted in Saline (1+9), 0.125 mL/100 g. The same dose is given again 1 hr. after dosing and 6-8 hrs. after dosing if the animals are kept overnight.

During the anaesthesia the rats are monitored continuously with special emphasis on skin colour and respiration. Since the use of opioids can cause loss of body heat (hypothermic), the rats are placed in the heating cupboard at 37° C. during the study.

The anaesthetised rats are placed on their backs on a thermic blanket or an insulation plate. A midline incision is made in the skin. A 2 cm incision into the abdominal cavity is made along the linea alba starting approximately 1 cm below the xiphoid cartilage (processus xiphoideus). A section of the jejunum approximately 50-60 cm from the pylorus that makes a significant bend is indentified below a layer of abdominal fat. The test compounds (0.2 mL per animal) are delivered to the jejunum by intra-intestinal injection using a G27 syringe. After dosing the needle is withdrawn carefully and the injection hole closed with a droplet of Histoacryl®. After 30 sec. the intestine is returned into the abdomen and the abdominal wall is closed by surgical sutures (Vicryl 4-0) ensuring that the wound ends are matching closely together. The skin is closed using wound clips. After the wound has been closed the rat is given 2 mL Saline s.c. in the flank to avoid dehydration and is placed in a special recovery cage under a heating lamp.

When the animals have completely recovered from the anaesthesia they are placed in the normal cage in the heating cupboard.

200 μL blood samples are collected from the tail vein from all animals at the following time points: Predose, 0.5, 1, 2, 3, 4 and 6 hrs. post injection. Blood samples are collected in test tubes containing EDTA 8 mM. Blood samples are kept on ice for max 20 min. before centrifugation (4000 rpm, 4° C., 10 min.). Plasma samples are collected immediately and stored at −20° C. until assayed. Plasma samples are analysed for test compounds. Test compound concentrations are determined by a sandwich ELISA using a guinea pig anti-hGH polyclonal antibody as catcher, and biotinylated hGH binding-protein (soluble part of human GH receptor) as detector. The limit of detection of the assay is 0.2 nM.

For intra-intestinal administration the compounds are dissolved to 1.5 mM in Glycine 20 mg/mL, Mannitol 2 mg/mL, NaHCO₃ 2.4 mg/mL, pH adjusted to 8.2.

### Method 11—Caco-2 Permeability Assay

Caco-2 cells has been used for measuring transport of test compounds across a monolayer of cells. The Caco-2 cell line is derived from a human colorectal carcinoma and is videly use for prediction of drug absorption across the human intestine. A cell layer of differentiated Caco-2 cells resemple the small intestinal columnar epithelium and is therefore usefull for testing transport properties of compound. The apparent permeability coefficients (Papp) obtained from Caco-2 cell transport studies have been shown to correlate to human intestinal absorption (J. D Irvine et al. J. Pharm. Sci. (1999) 88 and P. Artursson. J. Pharm. Sci. (1990) 79). The assay is performed culturing the cells on semi-permeable membrane. Test compounds are added to the apical side of the cell layer and their appearance on the basolateral side is measured over time and the calculated Pₐₚₚ represents intestinal absorption.

### Materials

Caco-2 cells were obtained from the American Type Culture Collection ATCC (Manassas, Va., USA). All cell culture reagents were purchased from LONZA BioWhittaker, unless otherwise noted. Trypsin-EDTA 0.25% from Invitrogen, Gibco (Denmark). MEM (non-essential amino acids) from Invitrogen, Gibco (Denmark), FBS HI from Invitrogen, Gibco (Denmark) and Hanks' Balanced Salt Solution (HBSS) Cat no. 14025 and HEPES Cat no. 15630, were purchased from Invitrogen, Gibco (Denmark). Mannitol, D-[1-3H(N)]-, was purchased from Perkin Elmer Danmark A-S. Microscint 40, scintillation fluid, was obtained from Packard BioScience (Groningen, The Netherlands).

### Cell Culture

Caco-2 cells were seeded in culture flasks and passaged (using DPBS and Trypsin 0.25%-EDTA) in Dulbecco's Modified Eagle's medium (DMEM) with Ultraglutamine, supplemented with 10% fetal bovine serum, penicillin-streptomycin (100 U/mL and 100 μg/mL, respectively), and 1% nonessential amino acids. The cells (between passages 23-40) were seeded onto tissue culture-treated Transwells™ (Costar, N.Y., USA) at a density of 10⁵ cells/cm². Transport experiments were subsequently performed on days 22-24 after seeding. Caco-2 cell monolayer cultures were grown in an atmosphere of 5% CO₂-95% O₂ and 90% humidity at 37° C. Growth media were replaced every other day. TEER was measured in Ω cm², at 37° C., using an epithelial voltohmmeter (Millicell®-ers; Millipore, Denmark), the mature Caco-2 cell monolayer exhibited a TEER.>600 Ωcm² prior to use in transport experiments.

### Permeability Assay

The integrity of the Caco-2 was evaluated via measurement of Mannitol, D-[1-³H(N)]—, (0.8 μCi/mL) Pₐₚₚ and monitoring the change in TEER over the course of a 2 h experiment. Prior to the experiment, growth media was removed from the mature Caco-2 cells and the monolayers were rinsed once with 37° C. HBSS. All compound transport experiments were carried out using a 200 μM solution of the test compound and the apical volume was 400 μL and the basolateral volume was 1000 μL. In the transport experiments, test compounds were added in the apical compartment at pH 7.4 and 200 μL was removed from the basolateral compartment at 15, 30, 45, 60 and 120 min, and the basolateral compartment was then replenished with 200 μL of fresh, preheated buffer. At 120 min, a 2×10 μL sample was taken from the donor compartment in order to establish the concentration of compound at the end of the experiment. All experiments were conducted at 37° C. for 2 h. Samples containing radioactive controls Mannitol, D-[1-³H(N)], were analyzed using TopCount NXT from Packard, while samples containing test compound were analyzed using Luminescence Oxygen Channeling Immunoassay (LOCI), which is a homogenous bead based assay. LOCI reagents include two latex bead reagents and a biotinylated antibody, which is one of the antibodies in the sandwich. One of the bead reagents was a generic reagent (donor beads) coated with streptavidin and containing a photosensitive dye. The second bead reagent (acceptor beads) was coated with the other antibody making up the sandwich. During the assay the three reactants combine with analyt to form a bead-aggregate-immune complex. Illumination of the complex releases singlet oxygen from the donor beads which channels into the acceptor beads and triggers chemiluminescence which was measured in the EnVision plate reader. The amount of light generated was proportional to the concentration of test compound. Sample/calibrator/control were diluted 10× in assay buffer. 1 μL diluted sample/calibrator/control was applied in 384-well LOCI plates. 15 μL of a mixture of biotinylated mAb HGH-7 (ES 7) and mAb HGH-3A7 (ES3)-conjugated acceptor-beads was added to each well (21-22° C.). The plates were incubated for 1 h at 21-22° C. 30 μL streptavidin coated donor-beads (67 μg/mL) was added to each well and all were incubated for 30 minutes at 21-22° C. The plates were read in an Envision plate reader at 21-22° C. with a filter having a bandwidth of 520-645 nm after excitation by a 680 nm laser. The total measurement time per well was 210 ms including a 70 ms excitation time.

### Calculation of Permeability

The accumulated amount of test compound appearing in the basolateral compartment over time, dQ/dt, was used to calculate the Pₐₚₚ using the following equation: Pₐₚₚ=dQ/dt×1/(AC₀), where A is the area of the filter (1 cm²) and C₀ is the initial concentration in the donor compartment.

### Preparation of Bile Acid Linkers

### Linker 1: -NN-AU-0087

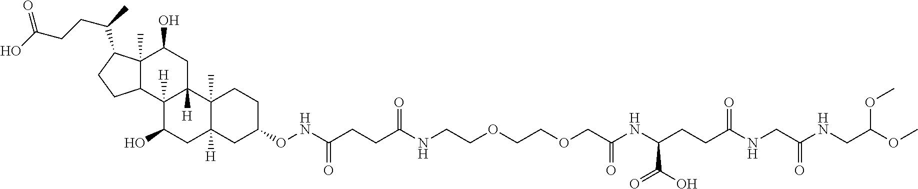

### Materials

Solid Support: PAM resin 0.7-1.3 mmol/g, 100-200 mesh, Sigma. Fmoc-8-amino-3,6-dioxaoctonic acid, MSNT (1-(Mesitylene-2-sulfonyl)-3-nitro-1,2,4-triazole) (from Chemimpex), Fmoc-Glu-Otu (Novabiochem), Fmoc-Gly-OH (GL Biochem), cholic acid (Sigma).

### Attachment of Fmoc-Gly-OH to PAM Resin

PAM resin (3 g, 3.3 mmol) was swelled in dry DCM (distilled over P₂O₅) for 1 hr. Fmoc-Gly-OH (4.9 g, 5 eq, 16.5 mmol), MSNT (4.9 g, 5 eq, 16.5 mmol), N-methyl imidazole (0.75 mL, 3.75 eq, 12.4 mmol) was dissolved in dry DCM (20 mL) containing 2-3 drops of THF. This solution was then added to the pre-swelled resin. The resin was allowed to rotate in a Robbins scientific rotator at RT. After 2 hrs. of reaction the resin was filtered and washed with DCM (6×10 mL) and DMF (6×10 mL). The process was repeated once again in identical conditions.

Removal of Fmoc group of Fmoc-Gly-PAM:

The Fmoc group of the Fmoc-Gly-PAM was deprotected by treating it twice with a solution of 20% (v/v) piperidine/DMF (15 mL) for 5 and 15 min. respectively. The resin was then filtered and washed with DMF (10×5 mL), DCM (6×10 mL) and DMF (6×10 mL). The deprotection of Fmoc group was confirmed by positive ninhydrin test.

### General Procedure for Successive Coupling of Fmoc-Amino Acids/Linker/Cholic Acid.

The Fmoc-deprotected amine resin was swelled in DMF. Pre-activated solution of Fmoc protected amino acid (5 equiv. respective to resin loading) HOBt (5 equiv.) and DIC (5 equiv.) in dry DMF was added to the pre-swelled resin and the reaction mixture was rotated at RT for 3 hrs. The resin was filtered and washed with DMF (6×5 mL), DCM (6×5 mL) and DMF (6×5 mL). The completion of coupling reaction was confirmed by negative ninhydrin test.

### General Procedure for Coupling of Cholic-Succinic Acid.

The Fmoc-deprotected amine resin was swelled in DMF. Pre-activated solution of cholic-succinic acid (3 equiv. respective to resin loading), HOBt (3 equiv.) and DIC (3 equiv.) in dry DMF was added to the pre-swelled resin and the reaction mixture was rotated at RT for 16 hrs. The resin was filtered and washed with DMF (6×5 mL), DCM (6×5 mL) and DMF (6×5 mL). The completion of coupling reaction was confirmed by negative ninhydrin test.

Cleavage of t-Butyl Groups from Peptidyl Resin

After the sequential attachment of target molecule to solid phase, the resin was washed with ether (6×10 mL) and dried in a vacuum desiccator overnight. The cleavage of the peptides from the solid support was achieved by treating the peptide-resin with a cleavage cocktail consisting of 95% TFA, 2.5% TIS, 2.5% water (25 mL) at RT for 1 hr. The resin was then washed with DCM (2×5 mL), DMF (2×5 mL), DCM (2×5 mL). The dried t-butyl side chain cleaved peptide resin was treated with a mixture of CHCl₃/aminoacetaldehydedimethylacetal (54:36) mL at 45° C. for 26 hrs. After cleavage the resin was drained and washed with DCM, MeOH, H₂O, 0.1% TFA, HFIP, DCM, MeOH and DCM. Filtrates were polled and evaporated in vacuum and the resulting oil dissolved in a given volume of distilled water. The pH was adjusted to 7 with TFA and the solution was freezed and lyophilized to yield crude product. The crude product was purified by preparative HPLC affording 263 mg of the compound which identity was confirmed by LC-MS.

### Purification and Characterization of Peptide

Analytical (Agilent 1100 series)

### Column: Zorbax Eclipse XDB-C₁₈ (4.6×150 mm, 5 μm).

### Temp: 25° C.

### Solvent A: 0.1% TFA/H₂O.

### Solvent B: 9:1:0.1 (MeCN/H₂O/TFA).

Flow rate 1 mL/min.
Wavelength monitored: 220 nm (Diode array detector).

### Gradient

| Time (min) | 0 | 2 | 15 | 20 | 25 | 30 | 32 | 35 |
|---|---|---|---|---|---|---|---|---|
| % B | 2 | 2 | 70 | 95 | 100 | 100 | 2 | 2 |

Crude peptide was dissolved in a mixture of MeCN/H₂O (1:1).

Preparative HPLC (Agilent 1100 series)

### Column: Zorbax-Eclipse XDB-C₁₈, 9.4×250 mm, 5 μm.

### Temp: 25° C.

### Solvent A: 0.005% TFA/H₂O.

### Solvent B: MeCN.

Wavelength monitored: 220 nm (Diode array detector).

### Gradient

| Time (min) | 0 | 2 | 50 |
|---|---|---|---|
| % B | 25 | 30 | 80 |

| Linker # | HPLC purity | LCMS |
|---|---|---|
| 1 | 87.3 (+12.6% | 942.1(M + 1) and |
| | aldehyde) | 964.3(M + Na) |

### Synthesis of Cholic Acid t-Butyl Ester—Succinic Acid

### Step-1: Synthesis of cholic acid t-butyl ester from cholic acid

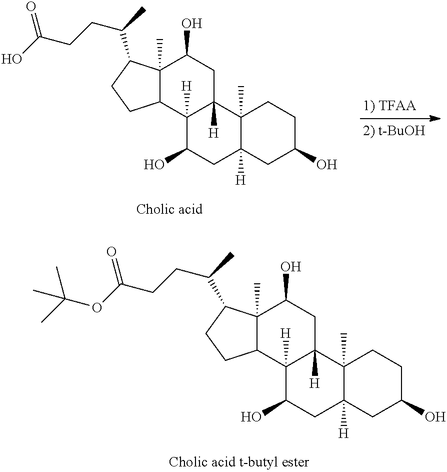

Trifluoroacetic anhydride (200 mL, 1.43 mol) was added over 5 min. to a stirred solution of cholic acid (50 g, 0.123 mol) in dry THF (1 L) at ice-bath temperature. The ice bath was removed and the mixture stirred for 80 min. at which time it was re-cooled and tert-butanol (300 mL, 3.16 mol) added. After stirring for 7 hrs at RT, aq. ammonia (280 mL, 25% w/w) was added with cooling, keep the temperature below 20° C., and the solution was left for 12 hrs. at RT. Another portion of aq. ammonia (100 mL) was added and after a further 4 hrs at RT the mixture was partitioned between diethyl ether (2 L) and water (1 L). (The progress of trifluoroacetate hydrolysis is monitored by TLC with EtOAc as eluent RF=0.4). The organic phase was washed with 2N NaOH solution (6×1 L), water (6×1 L), brine (1 L) and dried over sodium sulphate. The organic phase was filtered and the volatiles evaporated under reduced pressure affording cholic acid tert-butyl ester as a solid (54 g, 95%), pure by TLC and ¹H-NMR.

### Step-2: Synthesis of Cholic-ONH₂ from Cholic Acid Tert Butyl Ester

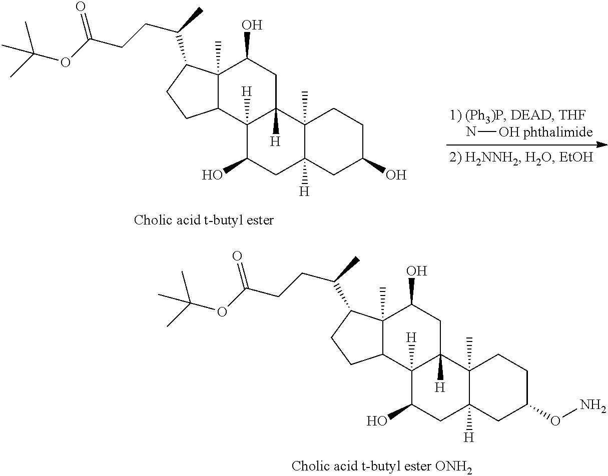

Cholic tert-butyl ester (15 g, 32.3 mmol) was dissolved in THF (150 mL). Triphenyl phosphine (16.9 g, 2 eq, 64.6 mmol) and N-hydroxyphthalimide (10.5 g, 2 eq, 64.6 mmol) was added to the reaction mixture at 0° C. DEAD (13 mL, 2.5 eq, 80.8 mmol) was added dropwise over a period of 1 hr. After complete addition the reaction mixture was allowed to reach RT and stirred over night. The volatiles were removed under reduced pressure. The residual oil was dissolved in EtOH (100 mL), hydrazine hydrate (12 mL, 247.3 mmol) was added and the reaction mixture was heated at 45° C. for 3 hrs at which point TLC indicated completeness of the reaction. The volatiles were removed under reduced pressure to give a pale solid which was washed with DCM (2×500 mL). The filtrate was concentrated under reduced pressure to give a yellow solid. The crude material was purified by coloumn chromatography using silica gel (60-120 mesh). The compound eluted at 1-2% MeOH in DCM as an eluent.

Step-3: Synthesis of Cholic Acid t-Butyl Ester—Succinic Acid from Cholic Acid t-Butyl Ester-ONH₂

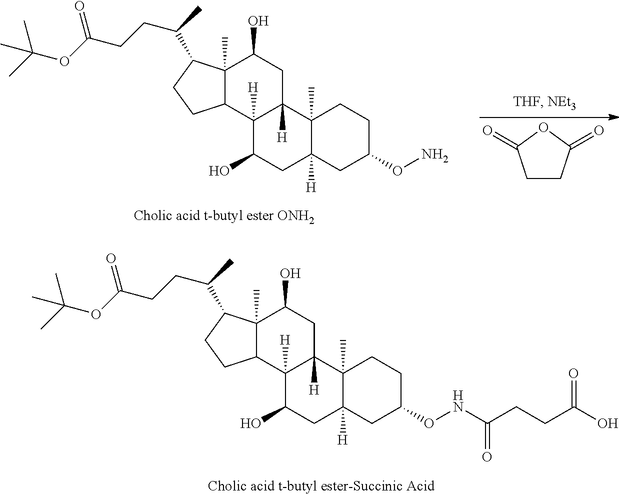

Cholic acid t-butyl ester-ONH₂ (20 g, 41.7 mmol) was dissolved in THF (200 mL). Succinic anhydride (4.6 g, 1.1 eq, 45.9 mmol), triethylamine (11.5 mL, 2 eq., 83.5 mmol) was added to reaction mixture, stirred for 3 hrs at RT. TLC indicated the completeness of reaction. THF was removed under reduced pressure to give viscous oil. The oil was dissolved in EtOAc (200 ml) and washed with citric acid solution, water, brine, dried over sodium sulphate and concentrated under reduced pressure to give a crude solid (30 g). The crude product was purified by recrystallization by dissolving in co-solvents of AcOEt/MeOH to yield 10 g (42%) of the compound with 95% purity.

The compound identity was confirmed by LC-MS and NMR.

### Linker 2:

In a similar way as described in relation to Linker 1 above the following compound was prepared using 4,4-dimethoxybutylamine instead of aminoacetaldehydedimethylacetal.

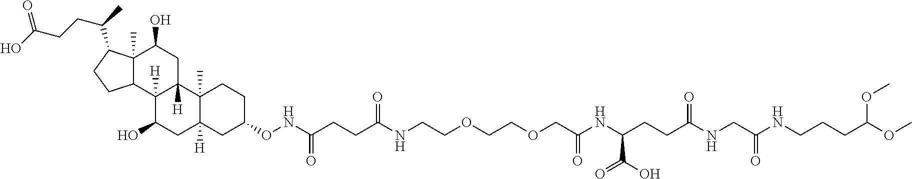

TOF-MS: mass 970.93

### Linker 3: -NN-AU-0090

In a similar way as described above in relation to Linker 1 the following compound was prepared.

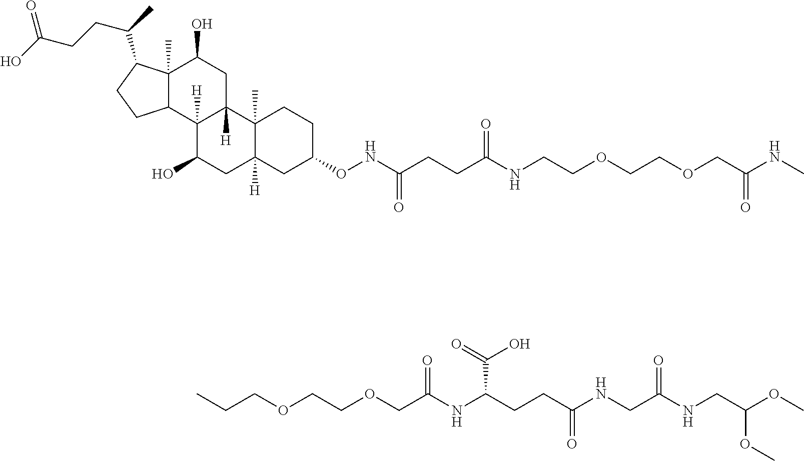

TOF-MS: mass 1087.28

### Linker 4: (N,N-AU-0109)

In a similar way as described in relation to Linker 1 above the following compound was prepared using 4,4-dimethoxybutylamine instead of aminoacetaldehydedimethylacetal.

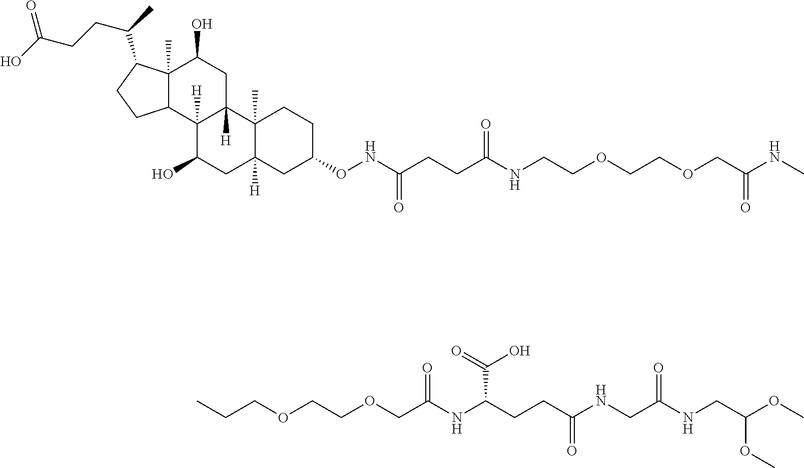

TOF-MS: mass 1115.34

### Linker 5: -(NN-AU-0091)

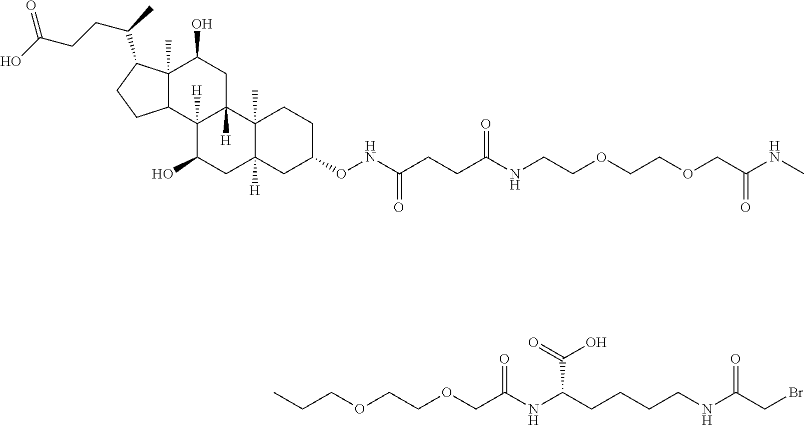

### Materials

Solid Support: Chlorotrityl chloride resin (CTC) 1.01 mmol/g, 200-400 mesh. Fmoc-8-amino-3,6-dioxaoctonic acid, Fmoc-Lys(Dde)-OH (Chemimpex).

Attachment of Fmoc-Lys(Dde)-OH to polymeric support:

Chlorotrityl chloride resin (CTC) (4 g, 4 mmol) was swelled in dry DCM (distilled over P₂O₅) for 1 hr. Fmoc-Lys(Dde)-OH (2.6 g, 1.2 eq, 4.8 mmol) and DIPEA (3.52 mL, 4.8 eq, 19.2 mmol) were dissolved in dry DCM (25 mL). This solution was transferred to the resin. The resin was allowed to rotate over night in a Robbins scientific rotator at RT. The reaction was filtered and washed with DCM (6×10 mL) and DMF (6×10 mL).

Removal of Fmoc group of CTC-Lys(Dde)-Fmoc resin:

The Fmoc group of the CTC-Lys(Dde)-Fmoc was deprotected by treating it twice with 20% (v/v) piperidine/DMF solution (20 mL) for 5 and 15 min. The resin was then filtered and washed with DMF (10×5 mL), DCM (6×10 mL), and DMF (6×10 mL). The deprotection of Fmoc group was confirmed by positive ninhydrin test.

General procedure for successive coupling of Fmoc-amino acids/linker:

The Fmoc-deprotected amine resin was swelled in DMF. Pre-activated solution of Fmoc protected amino acid (3 equiv. respective to resin loading), HOBt (3 equiv.) and DIC (3 equiv.) in dry DMF was added to the pre-swelled resin and the reaction mixture was rotated at RT for 3 hrs. The resin was filtered and washed with DMF (6×5 mL), DCM (6×5 mL) and DMF (6×5 mL). The completion of coupling reaction was confirmed by negative ninhydrin test. The successive amino acids are coupled in a similar fashion with deprotection and coupling cycles.

Coupling of Cholic Acid t-Butyl Ester—Succinic Acid:

The Fmoc-deprotected amine resin was swelled in DMF. Pre-activated solution of cholic acid t-butyl—succinic acid (3 equiv. respective to resin loading), HOBt (3 equiv.) and DIC (3 equiv.) in dry DMF was added to the pre-swelled resin and the reaction mixture was rotated at RT overnight. The resin was filtered and washed with DMF (6×5 mL), DCM (6×5 mL) and DMF (6×5 mL). The completion of coupling reaction was confirmed by negative ninhydrin test.

Deprotection of Dde from Peptidyl Resin:

The Dde group of the peptidyl resin was deprotected by treating it twice with 3% NH₂—NH₂ in DMF (20 mL) for 5 and 15 min. The resin was then filtered and washed with DMF (10×5 mL), DCM (6×10 mL), and DMF (6×10 mL). The deprotection of Dde group was confirmed by positive ninhydrin test.

### Coupling of Bromoacetic Acid:

The free amine resin was swelled in DMF. Pre-activated solution of bromoacetic acid (5 equiv. respective to resin loading), HOBt (5 equiv.) and DIC (5 equiv.) in dry DMF was added to the free amine resin and the reaction mixture was rotated at RT for 3 hrs. The resin was filtered and washed with DMF (6×5 mL), DCM (6×5 mL) and DMF (6×5 mL). The completion of coupling reaction was confirmed by negative ninhydrin test.

### Cleavage

Step I: Cleavage from Solid Phase as Protected Peptide:

After the final coupling of bromoacetic acid, the resin (4 g) was washed with ether (6×10 mL) and dried in a vacuum desiccator overnight. The cleavage of the peptides from the solid support was achieved by treating the peptide-resin with a mixture of hexafluoroisopropanol:DCM (1:4, 40 mL) at RT for 3 hrs. Cleavage mixture was collected by filtration and the resin was washed with DCM (2×5 mL). The excess reagent was concentrated to small volume under nitrogen and a small amount of DCM (5-10 mL) was added to the residue and evaporated under nitrogen. The process was repeated 3-4 times to remove most of the volatiles. The residue was cooled to 0° C. and anhydrous diethyl ether was added to precipitate the peptide. The precipitated peptide was centrifuged and the supernatant diethyl ether was removed and fresh diethyl ether was added to the peptide and re-centrifuged. The process was repeated 3× to remove all the organics affording 3.0 g of crude compound with 40% purity.

### Step II: Cleavage of Side Chain Protecting Groups:

Final de-protection of t-butyl groups was achieved by treating the crude compound with a cleavage cocktail consisting of 25% TFA, and 2.5% TIPS in DCM, (20 mL). The solution was stirred for 30 min. The excess reagent was concentrated to a small volume under nitrogen and a small amount of DCM (5-10 mL) was added to the residue and evaporated under nitrogen. The process was repeated 3-4 times to remove most of the volatiles. The residue was cooled to 0° C. and anhydrous diethyl ether was added to precipitate the peptide. The precipitate was centrifuged and the supernatant diethyl ether phase was removed and fresh diethyl ether was added to the peptide and re-centrifuged. The process was repeated 3× to remove all the organics affording 2.7 g of crude peptide with purity ˜35%. The compound was purified by preparative HPLC affording pure compound.

### Purification and Characterization of Linker 5

Analytical (Agilent 1100 series)

### Column: Zorbax Eclipse XDB-C₁₈ (4.6×150 mm, 5 μm).

### Temp: 25° C.

### Solvent A: 0.1% TFA/H₂O.

### Solvent B: 9:1:0.1 (MeCN/H₂O/TFA).

Flow rate 1 mL/min.
Wavelength monitored: 220 nm (Diode array detector).

### Gradient

| Time (min) | 0 | 2 | 15 | 20 | 25 | 30 | 32 | 35 |
|---|---|---|---|---|---|---|---|---|
| % B | 2 | 2 | 70 | 95 | 100 | 100 | 2 | 2 |

Crude peptide was dissolved in a mixture of MeCN/H₂O (1:1) and injected.

Rₜ=15.45 min.

Preparative HPLC (Agilent 1100 series)

### Column: Zorbax-Eclipse XDB-C₁₈, 9.4×250 mm, 5 μm.

### Temp: 25° C.

### Solvent A: 0.1% TFA/H₂O.

### Solvent B: MeCN.

Flow rate 7 mL/min.
Wavelength monitored: 220 nm (Diode array detector)

### Gradient

| Time (min) | 0 | 5 | 15 | 17 |
|---|---|---|---|---|
| % B | 5 | 25 | 70 | 70 |

| Linker # | HPLC purity (%) | LC-MS |
|---|---|---|
| 5 | 95 | 1064.5 (M + 1) |

### Linker 6: (N,N-AU-0078)

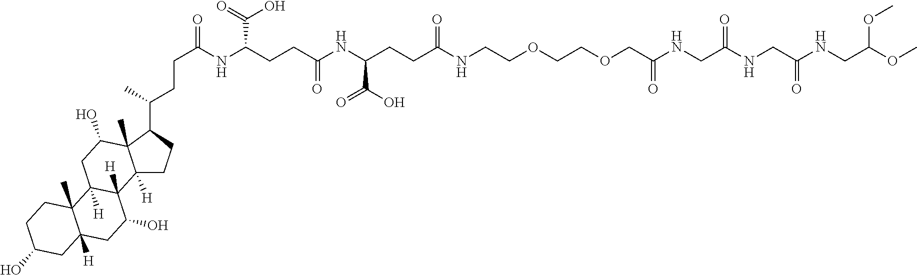

### Materials

Solid Support: PAM resin 0.7-1.3 mmol/g, 100-200 mesh (Sigma). Fmoc-8-amino-3,6-dioxaoctonic acid, MSNT (Chemimpex), Fmoc-Glu-O^{t}Bu (Novabiochem), Fmoc-Gly-OH (GL Biochem).

### First Attachment of Fmoc-Gly-OH to PAM Resin:

PAM resin (3 g, 3.3 mmol) was swelled in dry DCM (distilled over P₂O₅) for 1 hr. Fmoc-Gly-OH (4.9 g, 5 eq, 16.5 mmol), MSNT (4.9 g, 5 eq, 16.5 mmol), N-methyl imidazole (0.75 mL, 3.75 eq, 12.4 mmol) was dissolved in dry DCM (20 mL) containing 2-3 drops of THF and added to the pre-swelled resin. The resin was allowed to rotate in a Robbins scientific rotator at RT. After 2 hrs. of reaction the resin was filtered and washed with DCM (6×10 mL) and DMF (6×10 mL). The process was repeated once again in identical conditions.

Removal of Fmoc group of Fmoc-Gly-PAM:

The Fmoc group of the Fmoc-Gly-PAM was deprotected by treating it twice with 20% (v/v) piperidine/DMF solution (15 mL) for 5 and 15 min. The resin was then filtered and washed with DMF (10×5 mL), DCM (6×10 mL), and DMF (6×10 mL). The deprotection of Fmoc group was confirmed by positive ninhydrin test.

### General Procedure for Successive Coupling of Fmoc-Amino Acids/Linker/Cholic Acid:

The Fmoc-deprotected amine resin was swelled in DMF. Pre-activated solution of Fmoc protected amino acid (5 equiv. respective to resin loading) HOBt (5 equiv.) and DIC (5 equiv.) in dry DMF was added to the pre-swelled resin and the reaction mixture was rotated at RT for 3 hrs. The resin was filtered and washed with DMF (6×5 mL), DCM (6×5 mL) and DMF (6×5 mL). The completion of coupling reaction was confirmed by negative ninhydrin test.

Cleavage of t-Butyl Groups from Peptidyl Resin:

After the sequential attachment of target molecule to solid phase the resin was washed with diethyl ether (6×10 mL) and dried in a vacuum desiccator overnight. Cleavage of the peptide from the solid support was achieved by treating the peptide-resin with a cleavage cocktail consisting of 95% TFA, 2.5% triisopropyl silane (TIS), 2.5% water (25 mL) at RT for 1 hr. The resin was washed with DCM (2×5 mL), DMF (2×5 mL) and DCM (2×5 mL). The dried t-butyl side chain cleaved peptide resin was treated with a mixture of CHCl₃ (54 mL) and aminoacetaldehydedimethylacetal (36 mL) at 45° C. for 26 hrs. After cleavage the resin was drained and washed with DCM, MeOH, H₂O, 0.1% TFA, HFIP, DCM, MeOH, and DCM. Filtrates were polled and evaporated under vacuum. The resulting oil was dissolved in distilled water and the pH adjusted to 7 with TFA. The solution was freezed and lyophilized to yield crude oil. The crude oil was purified by preparative HPLC affording 263 mg of the compound.

### Purification and Characterization of Peptide

### Analytical (Agilent 1100 Series)

### Column: Zorbax Eclipse XDB-C₁₈ (4.6×150 mm, 5 μm)

### Temp: 25° C.

### Solvent A: 0.1% TFA/H₂O.

### Solvent B: 9:1:0.1 (MeCN/H₂O/TFA).

Flow rate 1 mL/min.
Wavelength monitored: 220 nm (Diode array detector).

### Gradient

| Time (min) | 0 | 2 | 15 | 20 | 25 | 30 | 32 | 35 |
|---|---|---|---|---|---|---|---|---|
| % B | 2 | 2 | 70 | 95 | 100 | 100 | 2 | 2 |

Crude peptide was dissolved in a mixture of MeCN/H₂O (1:1) and injected.

Rₜ=14.37 min.

### Preparative HPLC (Agilent 1100 Series)

### Column: Zorbax-Eclipse XDB-C₁₈, 9.4×250 mm, 5 μm

### Temp: 25° C.

### Solvent A: 0.005% TFA/H₂O.

### Solvent B: MeCN.

Wavelength monitored: 220 nm (Diode array detector).

### Gradient

| Time (min) | % B |
|---|---|
| 0 | 25 |
| 2 | 30 |
| 50 | 80 |

| Linker # | HPLC purity | LCMS |
|---|---|---|
| 6 | 85.9 (+10% | 1013.5 (M + 1) and |
| | free aldehyde) | 1035.3 (M + Na) |

### Linker 7: (N,N-AU-0015)

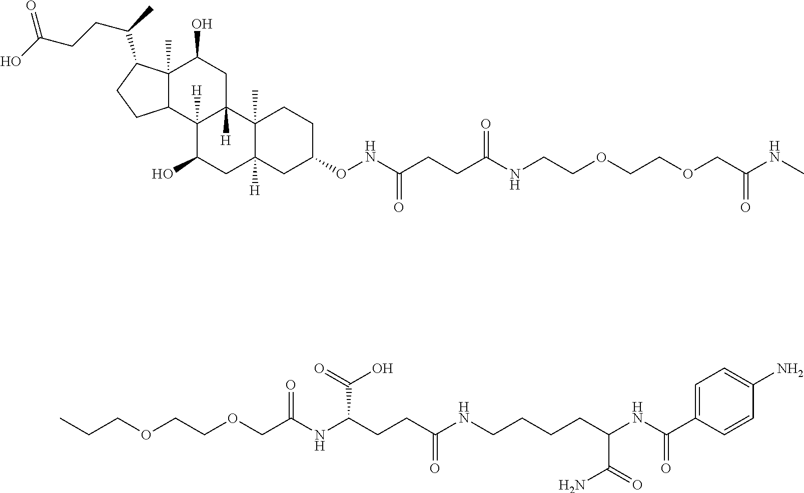

### Materials

Solid Support: 2 g. of Tentagel S RAM Resin 0.24 mmol/g,

Boc-4-aminobenzoic acid, H-Lys (Fmoc)-OH, HATU, NMM, DMF, Fmoc-Glu-OtBu, Fmoc-8-amino-3,6-dioxaoctonic acid (Chem Impex), succinic anhydride, cholic acid, DIC (Sigma),) TFA, Piperidine, DMF, DCM (Spectrochem Bombay, India) and HOBt, (Chemlabs, Leonid chemicals (Pvt) Ltd, India).

### Synthesis of Boc-Aminobenzoic-Lys (Fmoc)-OH

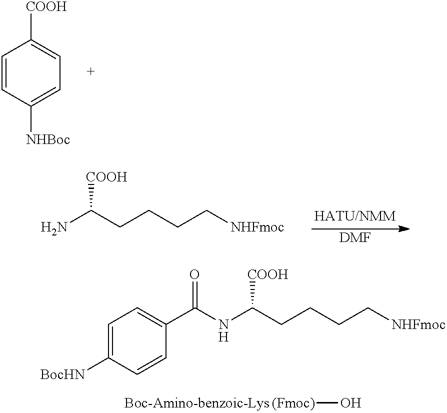

Boc-4-aminobenzoic acid (1.16 g, 4.8 mmol, 0.9 eq), HATU (2.05 g, 5.4 mmol, 1.0 eq) and NMM (1.2 mL, 10.8 mmol, 2 eq.) was dissolved in DMF (20 ml) and the reaction mixture was stirred at ice bath temperature for 5 min. To this mixture H-Lys(Fmoc)-OH (2 g, 5.4 mmol, 1.0 eq.) was added at ice bath temperature and the mixture was stirred at RT for an additional 3 hrs. After completion of the reaction (monitored by TLC) the reaction mixture was quenched with ice water and the precipitate filtered off. The precipitate obtained was further washed with ice water and hexane. The compound obtained after drying (2.1 g, 65%) was used without further purification in the next step.

### Attachment of Boc-Aminobenzoic-Lys (Fmoc)-OH to Polymeric Support

Tentagel S RAM resin (2 g, 0.24 mmol) was swelled in DCM and DMF for 1 hr each. Boc-aminobenzoic-Lys (Fmoc)-OH (1.4 g, 2.4 mmol), HOBt (327.6 mg, 2.4 mmol), DIC (371.6 mg, 2.4 mmol) was dissolved in DMF (10 mL) and transferred to the pre-swelled resin. The resin was allowed to rotate in a Robbins scientific rotator at RT for 5 hrs. The reaction was filtered and washed with DCM (6×10 mL) and DMF (6×10 mL).

Removal of Fmoc group of Boc-Aminobenzoic-Lys (Fmoc)-Resin:

The Fmoc group of the above Boc-aminobenzoic-Lys (Fmoc)-Resin was deprotected by treating it twice with 20% (v/v) piperidine/DMF solution (20 mL) for 5 and 15 min. The resin was then filtered and washed with DMF (10×5 mL), DCM (6×10 mL) and DMF (6×10 mL). The deprotection of the Fmoc group was confirmed by positive ninhydrin test.

### General Procedure for Successive Coupling of Fmoc-Amino Acids:

The Fmoc-deprotected amine resin was swelled in DMF. Pre-activated solution of Fmoc protected amino acid (3 equiv. respective to resin loading), HOBt (3 equiv.) and DIC (3 equiv.) in dry DMF was added to the pre-swelled resin and the reaction mixture was rotated at RT for 3 hrs. The resin was filtered and washed with DMF (6×5 mL), DCM (6×5 mL) and DMF (6×5 mL). The completion of coupling reaction was confirmed by negative ninhydrin test. The successive amino acids are coupled in a similar fashion with deprotection and coupling cycles.

### Coupling of Succinic Anhydride:

The Fmoc-deprotected amine resin was swelled in DMF. A solution of succinic anhydride (5 eq. respective to resin loading) with DIPEA (5 equiv.) in NMP was added to the pre-swelled resin and the reaction mixture was rotated at RT for 2 hrs. The resin was filtered and washed with DMF (6×5 mL), DCM (6×5 mL) and DMF (6×5 mL). The completion of coupling reaction was confirmed by negative ninhydrin test.

### Coupling of Cholic Hydroxyl Amine:

Cholic acid t-butyl ester-ONH₂ (5 equiv. respective to resin loading), HOBt (5 equiv.) and DIC (5 equiv.) in dry DMF was added to the pre-swelled resin and the reaction mixture was rotated at RT for 24 hrs. The resin was filtered and washed with DMF (6×5 mL), DCM (6×5 mL) and DMF (6×5 mL).

Cleavage of Peptide from the Resin:

After the sequential attachment of target molecule to solid phase, the resin was washed with diethyl ether (6×10 mL) and dried in a vacuum desiccator overnight. The cleavage of the peptide from the solid support was achieved by treating the peptide-resin with cleavage cocktail consisting of 95%12.5%12.5% (TFA/TIS/water) (25 mL) at RT for 3 hrs. The cleavage mixture was collected by filtration and the resin was washed with DCM (2×5 mL). The excess reagent was concentrated to small volume under nitrogen and a small amount of DCM (5-10 mL) was added to the residue and evaporated under nitrogen. The process was repeated 3-4 times to remove most of the volatiles. The residue was cooled to 0° C. and anhydrous diethyl ether was added to precipitate the peptide. The precipitated peptide was centrifuged and the supernatant diethyl ether phase was removed and fresh diethyl ether was added to the peptide and re-centrifuged. The process was repeated 3× to remove all the organics to affording crude compound (1.3 g, 15% purity). The target compound was purified by prep-HPLC affording the compound.

### Purification and Characterization of Peptide

### Analytical (Agilent 1100 Series)

### Column: Zorbax Eclipse XDB-C₁₈ (4.6×150 mm, 5 μm)

### Temp: 25° C.

### Solvent A: 0.1% TFA/H₂O.

### Solvent B: 9:1:0.1 (MeCN/H₂O/TFA)

Flow rate 1 mL/min.
Wavelength monitored: 220 nm (Diode array detector).

### Gradient

| Time (min) | 0 | 2 | 15 | 20 | 25 | 30 | 32 | 35 |
|---|---|---|---|---|---|---|---|---|
| % B | 2 | 2 | 70 | 95 | 100 | 100 | 2 | 2 |

Crude peptide was dissolved in MeCN/H₂O (1:1) and injected.

Rₜ=7.46 min.

### Preparative HPLC (Agilent 1100 Series)

### Column: Zorbax-Eclipse XDB-C₁₈, 9.4×250 mm, 5 μm.

### Temp: 25° C.

### Solvent A: 0.005% TFA/H₂O.

### Solvent B: MeCN.

Flow rate 5 mL/min.
Wavelength monitored: 220 nm (Diode array detector).

### Gradient

| Time (min) | % B |
|---|---|
| 0 | 5 |
| 40 | 30 |
| 15 | 40 |

| Linker # | HPLC purity (%) | LCMS |
|---|---|---|
| 7 | 96 | 1189.9 (M + 1) and |
| | | 1211.9 (M + Na) |

### Linker 8: (N,N-AU-0016) (N,N-AU-0016)

In a similar way as described in relation to Linker 7 above the following compound was prepared using FMOC-Lys(Mtt)-OH and Wang Resin.

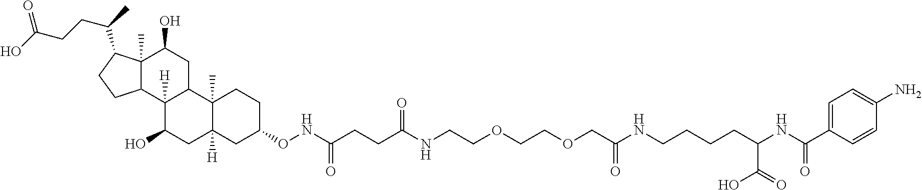

TOF-MS: mass 916.13

### Linker 9: (N,N-AU-0086)

In a similar way as described in relation to Linker 7 above the following compound was prepared using FMOC-Lys(Mtt)-OH and Wang Resin.

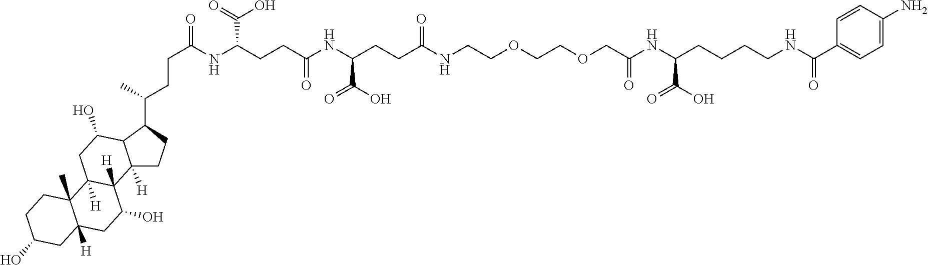

TOF-MS: mass 1059.4

### HPLC:

### Column: Zorbax Eclipse XDB-C₁₈ (4.6×150 mm, 5 μm)

### Temp: 25° C.

### Solvent A: 0.1% TFA/H₂O.

### Solvent B: 9:1:0.1 (MeCN/H₂O/TFA)

Flow rate 1 mL/min.
Wavelength monitored: 220 nm (Diode array detector).

### Rₜ=14.02 min.

### Linker 10:

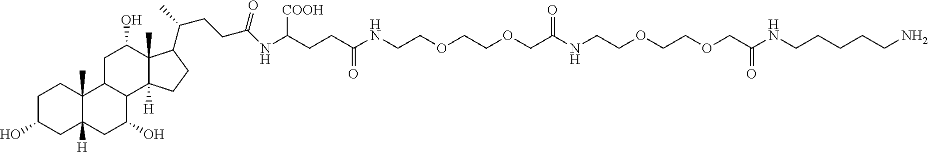 [SMILES: C[C@H](CCC(=O)NC(CCC(=O)NCCOCCOCC(=O)NCCOCCOCC(=O)NCCCCCN)C(=O)O)C1CC[C@H]2C3C(C[C@H](O)[C@]12C)[C@@]1(C)CC[C@@H](O)C[C@H]1C[C@H]3O]

### Materials

| | | Dens | Mw | Mol | n | W | V |
|---|---|---|---|---|---|---|---|
| | Name | [g/ml] | [g/mol] | Ratio | [mmol] | [mg] | [ul] |
| A | Cadaverin | | 102.18 | 10 | 10 | 1021.81 | |
| B | Product: | | 912.18 | | | | |
| | C₄₆H₈₁N₅O₁₃ | | | | | | |
| C | 2-chloro- | | | 1.1 | 1.1 | 1000 | |
| | tritylresin | | | | | | |
| D | Fmoc- | | 385.42 | 4 | 4 | 1541.68 | |
| | OEG-OH | | | | | | |
| E | DIC | 0.815 | 126.2 | 4 | 4 | 504.8 | 619 |
| F | HOBt | | 153.2 | 4 | 4 | 612.8 | |
| G | Fmoc- | | 385.42 | 4 | 4 | 1541.68 | |
| | OEG-OH | | | | | | |
| H | DIC | 0.815 | 126.2 | 4 | 4 | 504.8 | 619 |
| I | HOBt | | 153.2 | 4 | 4 | 612.8 | |
| J | Fmoc- | | 425.49 | 4 | 4 | 1701.96 | |
| | Glu-OtBu | | | | | | |
| K | DIC | 0.815 | 126.2 | 4 | 4 | 504.8 | 619 |
| L | HOBt | | 153.2 | 4 | 4 | 612.8 | |
| M | Cholic Acid | | 408.58 | 4 | 4 | 1634.32 | |
| N | DIG | 0.815 | 126.2 | 4 | 4 | 504.8 | 619 |
| O | HOBt | | 153.2 | 4 | 4 | 612.8 | |

### Procedure:

A 0.5M HOBt solution in NMP was prepared.

Resin was swelled for 30 min. in DCM.

Resin was treated with a mixture of cadaverin:DCM (10 mL, 1:9) for 30 min.

Resin was treated with 5% DIPEA; 5% MeOH in DCM for 30 min.

Resin was washed with DCM (3×)

### Coupling of Fmoc-OEG-OH:

A 0.5M solution of Fmoc-OEG-OH/DIC/HOBt in NMP (8 mL) was prepared and after 2 min. of mixing added to the trityl resin (1 g). Resin was shaken for 45 min. at RT. followed by washing with NMP (5×) and DCM (5×). A small amount of product was cleaved from the resin and analyzed by LCMS.

### Capping:

A solution of Ac₂O/DIPEA/NMP (1:1:5) was added. The resin was stirred at RT. for 10 min. and washed with NMP (5×) and DCM (5×).

### De-Fmoc:

Resin was treated with 30% piperidin-NMP for 2×10 min. Resin was washed with NMP (5×) and DCM (5×).

### Coupling of the Second Fmoc-OEG-OH:

A 0.5M solution of Fmoc-OEG-OH/DIC/HOBt in NMP (8 mL) was prepared and mixed for 2 min. and added to the above resin. The resin was shaken for 45 min. at RT. followed by washing with NMP (5×) and DCM (5×). A small amount of product was cleaved from resin and analyzed by LCMS.

### De-Fmoc:

Resin was treated with 30% piperidin-NMP for 2×10 min.

Resin was washed with NMP (5×) and DCM (5×).

### Coupling of Fmoc-Glu-tBu:

A 0.5M solution of Fmoc-Glu-tBu/DIC/HOBt in NMP (8 mL) was prepared and mixed for 2 min. and added to the resin. The resin was shaken for 45 min. at rt. and washed with NMP (5×) and DCM (5×). A small amount of product was cleaved from resin and analyzed by LCMS.

### De-Fmoc:

Resin was treated with 30% piperidin-NMP for 2×10 min.

Resin was washed with NMP (5×) and DCM (5×).

### Coupling of Cholic Acid:

A 0.5M solution of cholic acid/DIC/HOBt in NMP (8 mL) was prepared and mixed for 2 min. and added to the resin. The resin was shaken overnight at RT. followed by washing with NMP (5×) and DCM (5×). A small amount of product was cleaved from resin and analyzed by LCMS.

Cleavage from Resin:

Resin was treated twice with a mixture of TFA/water/Et₃SiH (15 mL, 90:5:5) for 10 min. Combined filtrates were concentrated and analyzed by LCMS. LCMS showed formation of product (60%) and trifluoroacetylated product (40%). The residual oil was therefore dissolved in 2M NH₃/MeOH (10 mL) and stirred for 1 hr at RT. The volatiles were removed in vacuum and the residue dissolved in a mixture of MeCN and MQ (11 mL, 1:10), filtered and purified by prep HPLC using Waters:

| | | | | | | |
|---|---|---|---|---|---|---|
| | Dens | Mw | Mol | n | W | V |
| Name | [g/ml] | [g/mol] | Ratio | [mmol] | [g] | [ml] |

| | | | |
|---|---|---|---|
| A | | 102.181 | 1 |
| B | Product: | 912.184 | |
| | C46H81N5O13 | | |

Fractions were combined and concentrated in vacuum affording 194 mg colourless oil. TOF-MS: mass 912.13

In a similar way as described in relation to Linker 5 above the following compounds were prepared using FMOC-Lys(Mtt)-OH and Wang Resin.

### Linker 11:

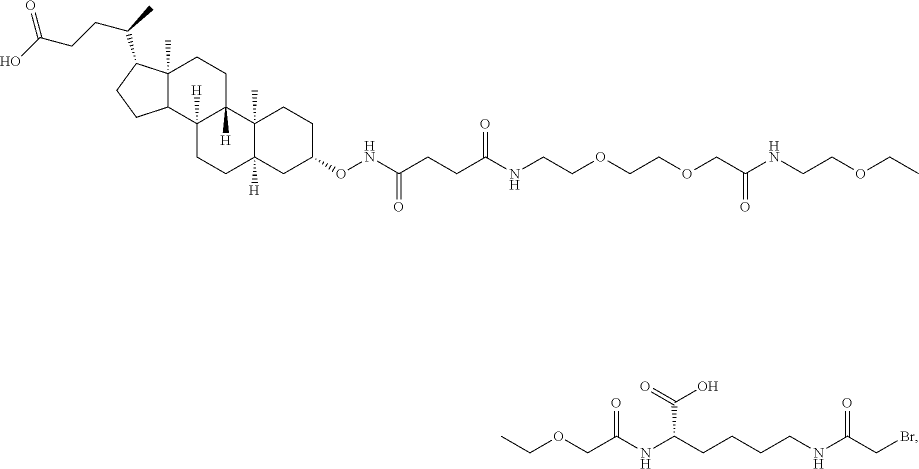

| Linker # | HPLC purity (%) | LC-MS |
|---|---|---|
| 11 | 96 | 1032.7 (M + 1) |

### Linker 12:

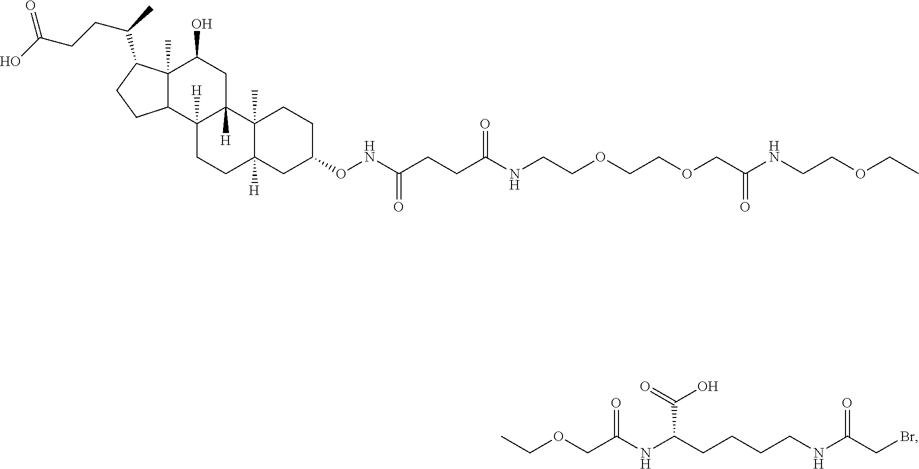

| Linker # | HPLC purity (%) | LC-MS |
|---|---|---|
| 12 | 94 | 1047.6 (M + 1) |

and

### Linker 13:

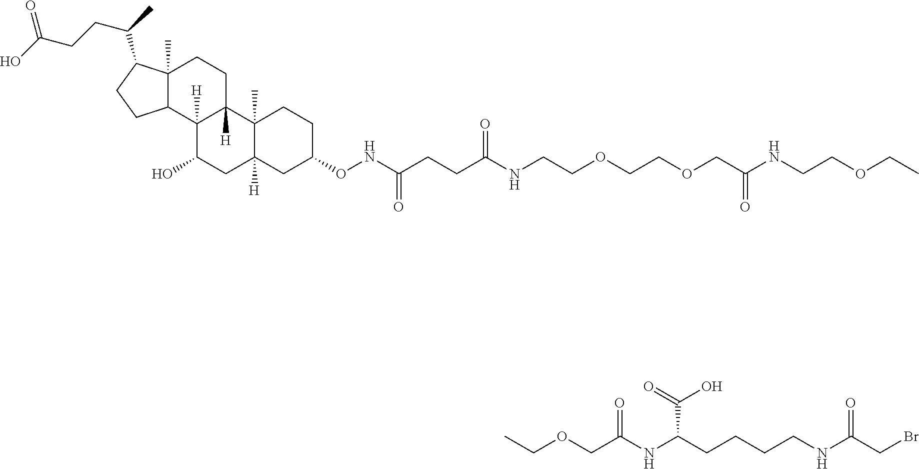

| Linker # | HPLC purity (%) | LC-MS |
|---|---|---|
| 13 | 95 | 1048.7 (M + 2) |

### Synthesis of Cholic Acid Conjugated Compound

Attachment of Linker 3 at N-terminal Phe according to Method 3.1.

### Compound A—NNC 0186-0000-0078

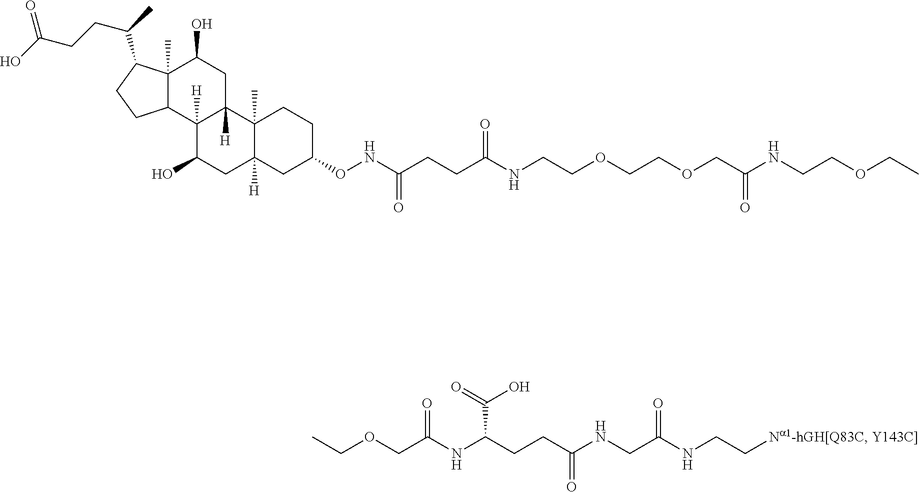

Reductive alkylation of hGH[Q83C, Y143C] NNC 0186-0000-0017 at the N-terminal with Linker 3.

### Sample:

| Compounds | Mw | Ratio | μmole | mg |
|---|---|---|---|---|
| hGH[Q83C, Y143C] | 22.038 g/mole | 1 | 6.8 | 150 |
| Linker 3 | 1.087 g/mole | 1.5 | 10.2 | 11 |
| NaCNBH₃ | 62.84 g/mole | 124 | 841 | 53 |

Freeze-dried hGH[Q83C, Y143C] (150 mg) was dissolved in 10 mM ammoniumbicarbonate buffer and buffer changed to 25 mM HEPES, pH 7.0 using Amicon Ultra15 devices (Ultracel 10K tubes) by centrifugation at 4000 rpm/min., 10° C., 3×8 min.

### Procedure:

Linker 3 (11 mg) was treated with TFA (500 μL) for 6 min., in order to liberate the aldehyde—and evaporated followed by stripping twice with EtOH (0.5 mL) to dryness.

The above solution of hGH[Q83C, Y143C] was added portion wise while keeping pH ˜7.0 by addition of 1N NaOH. NaCNBH₃ (53 mg) dissolved in water (500 μL) was added portions wise (5×100 μL) with intervals of 5 min.

The reaction flask was wrapped in tin-foil and the reaction mixture gently stirred at RT over night. Clear solution obtained after 1 hr. of stirring. After stirring over night a sample was analyzed by LC/MS: m/z of product: 23.063 at Rₜ=6 min. but also starting protein left.

The mixture was purified using combinations of IEX and HIC chromatography, buffer exchange on a G25 column and ultra-filtration affording 4 mg, 95% pure compound.

m/z of product: 23.063 [M+1].

### Compound B—NNC 0186-0000-0036

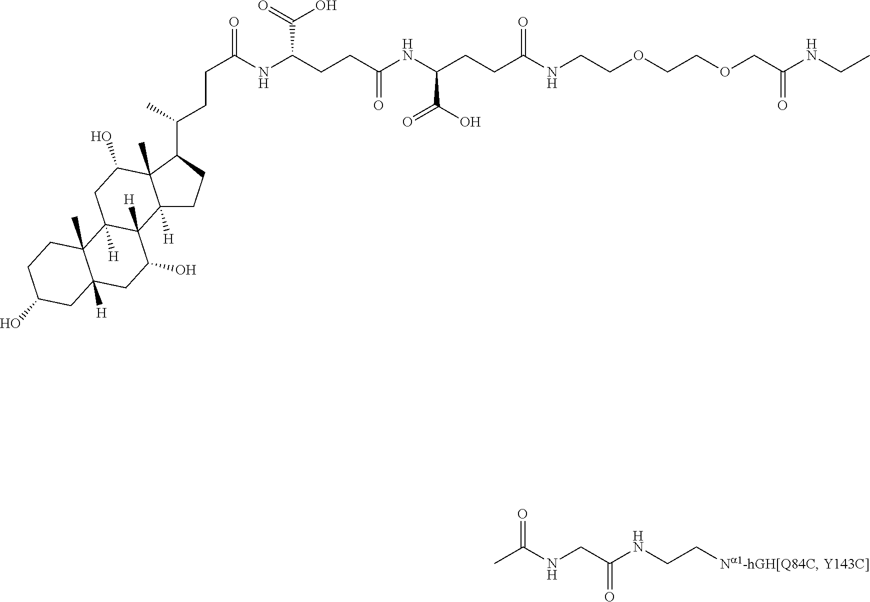

Reductive alkylation of hGH[Q83C, Y143C] at the N-terminal with Linker 6 according to Method 3.1.

### Sample:

| Compound | Mw | Ratio | μmole | mg |
|---|---|---|---|---|
| hGH[Q83C, Y143C] | 22.038 g/mole | 1 | 8.67 | 191 |
| Linker 6 | 1.013 g/mole | 4.5 | 39.0 | 39.5 |
| NaCNBH3 | 62.84 g/mole | 124 | 1075 | 67.6 |

Freeze dried hGH[Q83C, Y143C] (200 mg) was dissolved in 10 mM ammoniumbicarbonate buffer and buffer changed to 25 mM HEPES, pH 7.00 using Amicon Ultra15 devices (Ultracel 10K tubes) by centrifugation at 4000 rpm/min., 10° C., 3×7 minutes. Final volume: 40 mL.

### Procedure:

Linker 6 (39.6 mg) was treated with a mixture of 50% TFA/H₂O (2 mL) for 20 min., evaporated to dryness on a rotavapor and stripped with EtOH (2×1 mL).

1N NaOH (5 μL) was added to the dry linker to prevent a drop in pH, when adding hGH[Q83C, Y143C].

The above solution of hGH[Q83C, Y143C] was added to the linker and the pH adjusted to 7.00 with 1N NaOH (90 μL). The pH dropped to 6.55 during the addition of the reaction mixture. pH adjusted to 7.00 with 1N NaOH (40 μL).

NaCNBH₃ (67.6 mg) was dissolved in water (600 μL) and added portion wise (3×200 μL) with intervals of 5 min. The reaction mixture was wrapped in tin-foil and incubated over night with gently stirring. LCMS analysis indicated still some starting material. The reaction mixture was incubated and gently stirrer at RT and left over night at 4° C. The reaction mixture was pH adjusted to 7.50 and diluted with Milli-Q water to a conductivity of 20 mS, before purification on an Äkta system.

Sample volume: 110 mL

### Column: HiLoad 26/10 Q Sepharose

### Buffer A: 20 mM HEPES, pH: 7.50+10% Ethylenglycol

### Buffer B: 20 mM HEPES, pH: 7.50+μM NaCl+10% Ethylenglycol

Flow: 6 mL/min.

### Gradient: 0-5%, 1CV, 5-40%, 12CV, 40-100%, 1CV

Fraction size: 7 mL. The sample was applied using a sample pump with a flow rate at 5 mL/min. Fraction E4, desired mass found: 22.989

All fractions which were not pure enough were pooled for further purification.

Fraction E4 was buffer changed to 10 mM ammoniumbicarbonate buffer during 3 runs:

### Column: 50/30 Sephadex G25 Fine

Buffer A: 10 mM ammoniumbicarbonate buffer.
Flow: 10 mL/min.
Fraction size: 45 mL.
1. run: Fraction A2 to A3 pooled; 16.6 mg of pure compound
2. run: Fraction A1 to A2 pooled; 12.9 mg of pure compound
3. run: Fraction A7 to A8 pooled; 19.2 mg of pure compound

LC-MS (electro spray): m/z: 22.989 found

### Compound C 0186-0000-0050

Attachment of Linker 7 at Gln residue according to Method 3.2.

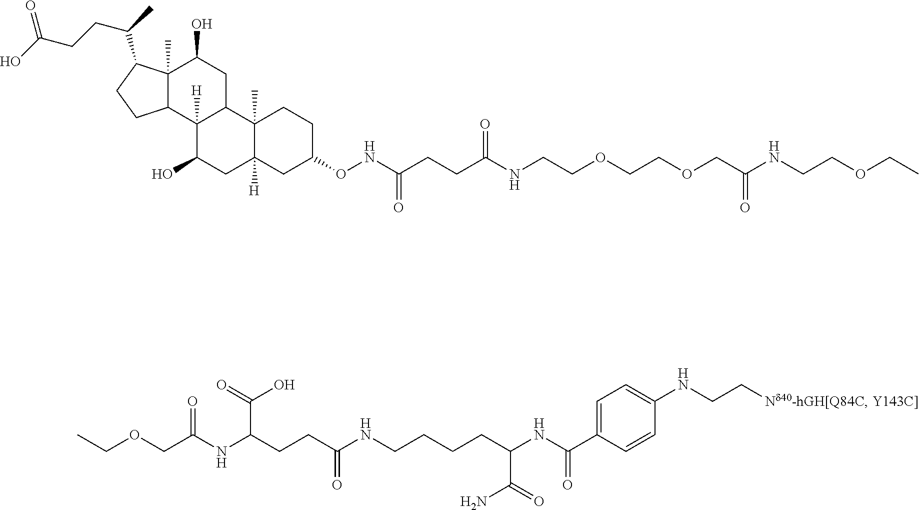

Transaminated hGH[Q83C, Y143C] was generated as described above and conjugation with Linker 7. TOF-LC-MS: mass 23.253, 99

Pool 1: Yield 12.8 mg. Purity: 100%.

Pool 2: Yield 19.2 mg. Purity: 100%.

### Compound D NNC 0186-0000-0061

In a similar way as described above in relation to Compound C, the following compound was prepared using linker 8.

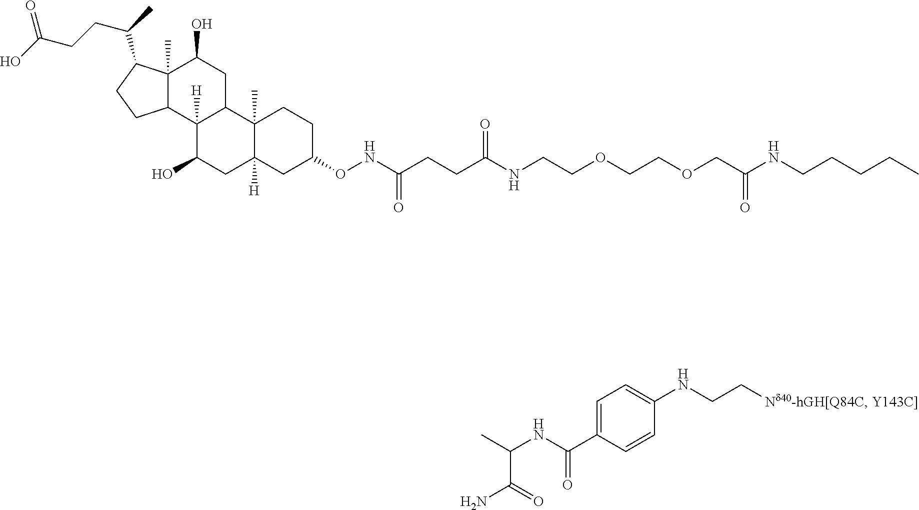

### Compound E—NNC 0186-0000-0081

Attachment of Linker 5 at position 101 (L101C) according to Method 3.3.

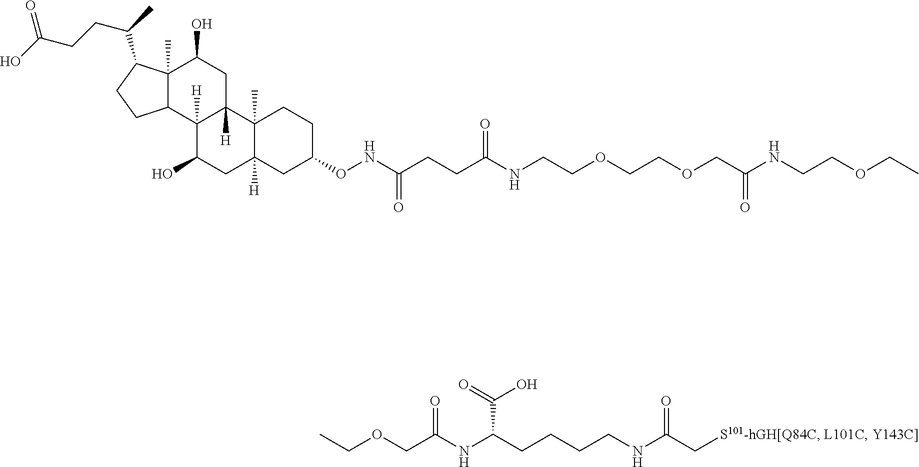

hGH[Q84C, L101C, Y143C] obtained as described in Method 2 had part of its free cysteine blocked with glutathione and cystamine. Selective deblocking was performed using TSPP in a TEA/EDTA buffer.

To a solution of hGH[Q84C, L101C, Y143C] in a 20 mM TEA-buffer, pH 7.4 with approximately 0.1M NaCl (170 mL, protein conc.: 2.69 mg/mL) was added EDTA (126 mg) to a concentration of 2 mM and the pH adjusted to 8.5 with 6N NaOH. TSPP (339 mg, 25 eq.) was added and the resulting mixture gently stirred at RT for 4 hrs.

LC-MS TOF: 22.028 correspond to deprotected protein (−75)

Coupling with Linker:

| Reagents | Mw (g/mole) | Ratio | μmole | mg |
|---|---|---|---|---|
| hGH[Q84C, L101C, Y143C] | 22.028 | 1 | 6.35 | 140 |
| Linker 5 | 1.063 | 5 | 32 | 33 |

To a solution of the deblocked hGH[Q84C, L101C, Y143C] (140 mg, 13.3 mL) in TRIS/NaCl was added a solution of Linker 5 (33 mg) dissolved in a mixture of NMP (1 mL), NaCl (0.88 mg) and Tricine/EDTA-buffer (14 mL). The resulting mixture was stirred gently at ambient temperature over night (16 hrs.).

LC-MS TOF: 23.010 found at Rₜ=6.6 min.

HPLC of crude product˜92% pure

Total amount: 138 mg.

### Compound F NNC 0186-0000-0087

In a similar way as described in relation to compound E the following compound was prepared using Linker 5 and the GH compound hGH[S62C, Q84C, Y143C].

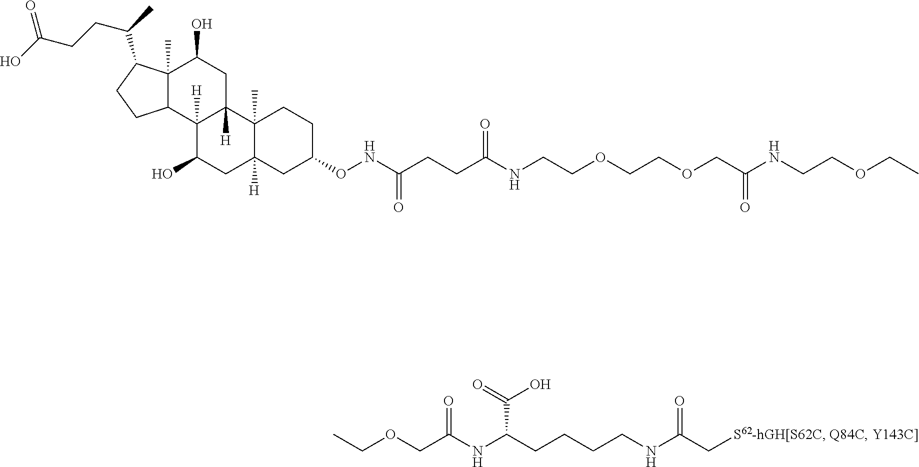

After coupling of protein and linker over night the reaction mixture was buffer changed to 20 mM triethenolamine+10% Ethylen glycol at pH 8.50.

### Buffer Change:

### Column: HiPrep 26/10 Desalting

Buffer A: 20 mM TEA, pH: 8.50+10% Ethylen glycol
Flow: 8 mL/min.
Fraction A3 and A5 were pooled for purification.

### Purification:

### Column: HiLoad 26/10 Q Sepharose HP

Buffer A: 20 mM Triethenolamine+10% Ethylen glycol, pH 8.50
Buffer B: 20 mM Triethanolamine+10% Ethylen glycol, pH 8.50+1M NaCl
Gradient 1: 0-10% Buffer B over 1 CV
Gradient 2: 10-40% buffer B over 12 CV
Gradient 3: 40-100% Buffer B over 1 CV
Flow: 6 mL/min.

### Temp: RT

### Fractions: 7 mL

Fractions D11 to D8 were pooled and buffer changed and
fractions C12 and D12 were pooled and buffer changed:

### Buffer Chance of D11 to D8:

### Column: HiPrep 26/10 Desalting

Buffer A: 10 mM ammoniumbicarbonate buffer
Flow: 8 mL/min.
Fraction A2, A4, A6 and B6 were collected and pooled affording 47.5 mg (HPLC quantification) of 100% pure compound.

### Buffer Chance of C12 and D12:

### Column: HiPrep 26/10 Desalting

Buffer A: 10 mM ammoniumbicarbonate buffer
Flow: 8 mL/min
Fraction B4 was collected affording 11.9 mg (HPLC quantification) of 90% pure compound.

LC-MS TOF: 23.036, 62 [M+1]

### Compound G NNC 0186-0000-0088

Attachment of Linker 10 at Gln40 of hGH[Q84C, Y143C] according to Method 3.2.

Direct transamination of a GH compound (I) with bile acid linker A-W—B1-NH₂ (II) and mTGase (as described in method 3 (2).

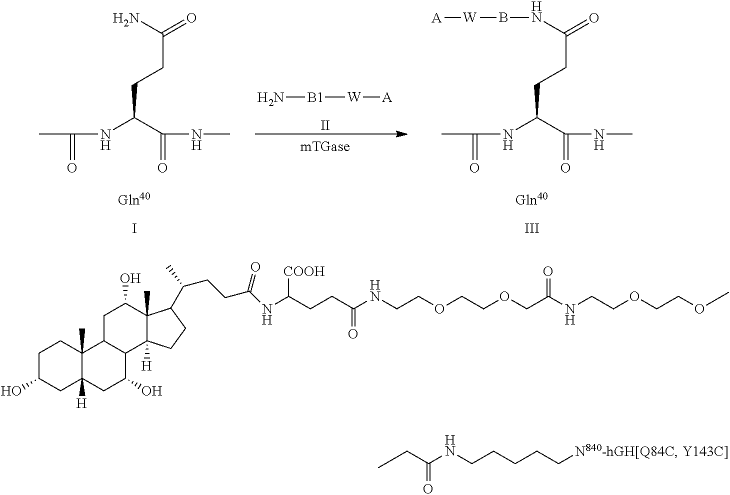

### Buffers:

Reaction buffer: 20 mM Triethanolamin, pH 8.5
Imidazol buffer: 20 mM imidazole buffer pH 7.4, 10 mM CaCl₂, 10% glycerol, 0.02% Tween 80.

### Solutions:

A: 227 uM solution of hGH[Q84C, Y143C] (4.4 mg) in reaction buffer
B: 2.91 mM solution of linker 10 in reaction buffer
D: Ajinomoto TGase (31 mg) in 20 mM imidazole buffer (515 μL), pH 7.4, 10 mM CaCl₂, 10% glycerrol, 0.02% Tween 80; C=60 mg/mL=11.4 μM.

Solutions A and B were mixed and D added at 25° C. according to scheme below. The resulting mixture was stirred for 42 hrs. at RT.

| hGH[Q84C, | | | Imidazol- | |
|---|---|---|---|---|
| Y143C] | Linker 10 | mTGase | buffer | Final vol. |
| A (μL) | B (μL) | D (μL) | (μL) | (μL) |
| 880 | 775 | 400 | 1945 | 4000 |

The reaction mixture was purified on an Äkta 100 system, using operating conditions described in table below:

| Column | HiTrap Q HP, CV = 5 mL |
|---|---|
| Buffer A1 | 20 mM HEPES, 10% ethylenglycol, pH = 7.5 |
| Buffer B1 | 20 mM HEPES, 10% ethylenglycol, 1M NaCl, |
| | pH = 7.5 |
| Flow | Variable (see below) |
| Wavelength | 214/280 nm |
| Max Pressure | 0.7 MPa (using FR-902 for back pressure) |
| Temperature | RT |
| Fraction volume | 1.5 mL for all using 96 wells microtiter plates |
| Injection volume | 5 mL |
| Equilibration | Predone, 100% A1 |
| Injection + | 2 CV, flow = 2 mL/min, 100% A1 |
| wash out | |
| Volume | 50 CV |
| Time | Approx. 85 min. |
| CV | Gradient; Flow |
| 16 | 0-12% B1; 3 mL/min. |
| 10 | 12-25% B1; 3 mL/min. |
| 10 | 25-50% B1; 3 mL/min. |

3 peaks were eluted with 9-18% buffer B1 in 40 mL. Fractions containing
target molecule (LC-MS 22.933) were collected.

### Compound H

In a similar way as described in relation to compound E the following compound was prepared using Linker 11 and the GH compound hGH[Q84C, L101C, Y143C].

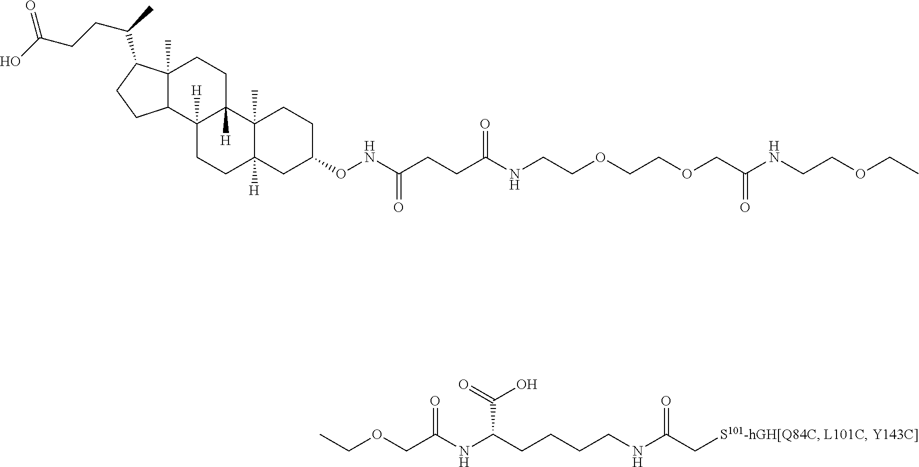

After coupling of protein and linker over night the reaction mixture was buffer changed to 20 mM triethenolamine+10% Ethylen glycol at pH 8.50.

### Buffer Chance:

### Column: HiPrep 26/10 Desalting

Buffer A: 20 mM TEA, pH: 8.50+10% Ethylen glycol
Flow: 8 mL/min.
Fractions were pooled for purification.

### Purification:

The reaction mixture was purified at an Äkta system.

Diluted with 20 mM TEA, pH 8.50+10% ethylenglycol to a salt concentration at 0.1M NaCl.

### Column: HiLoad 26/10 Q Sepharose HP

### CIP: 1M NaOH

Buffer A: 20 mM Triethenolamine+10% Ethylen glycol pH 8.50
Buffer B: 20 mM Triethanolamine+10% Ethylen glycol pH 8.50+1 M NaCl
Gradient 1: 0-14% Buffer B over 1 CV
Gradient 2: 14-19% buffer B over 10 CV
Gradient 3: 19-100% Buffer B over 1 CV
Flow: 5 mL/min

### Temp: RT

Fractions: 7 mL per fraction
Sample was applied using a sample pump with a flow of 5 mL/min:
Very broad peak.
Fractions E12, E7, F3, F8, G2, G5 analyzed at HPLC.
Fractions E7 through G7 were pooled.

### Volume: 190 mL

Quantified and checked before desalting at HPLC.

Area found: 3624836, inj.: 5 μL, ex. coef.:1000000=0.72 mg/mL×190 mL=137 mg before buffer change

### Product at RT.: 14.36 min.

### Desalting:

The product was buffer changed to 10 mM ammoniumbicarbonate buffer on an Äkta.

Column: HiPrep G25 Fine 50/20 desalting
Buffer A: 10 mM ammoniumbicarbonate
Flow: 8 mL/min

### Run: 1.5 CV

### Temp: RT

Fractions: 45 mL per fraction
Sample was applied using a sample pump with a flow 5 mL/min in 2 runs:
Fractions A3 to A5 from first run were pooled together with fractions A2 to A4 from the second run.

### Volume: 270 mL

The pools were concentrated using Amicon Filter devises with a cut off at 10 kDa. Centrifuged at 4000 rpm/6 min.

Total volume: 37.5 mL

### Quantified at HPLC:

Area found: 16576155, inj.: 5 μL, ex. coef.: 1000000=3.32 mg/mL×37.5 mL=124.5 mg

LC-MS (electrospray): m/z: 22.978, 4 at Rt.: 6 min.

HPLC: Rt=14.23 min,

### Compound I

In a similar way as described in relation to compound E the following compound was prepared using Linker 12 and the GH compound hGH[Q84C, L101C, Y143C].

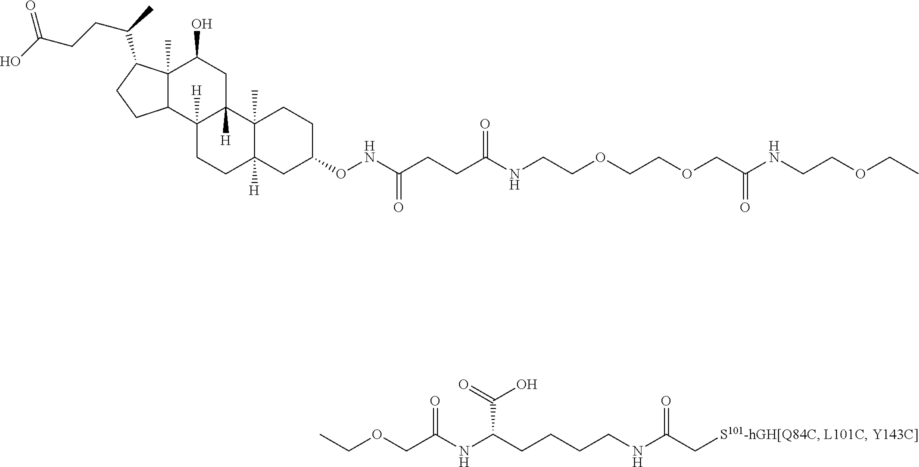

After coupling of protein and linker over night the reaction mixture was buffer changed to 20 mM triethenolamine+10% Ethylen glycol at pH 8.50.

### Buffer Change:

### Column: HiPrep 26/10 Desalting

Buffer A: 20 mM TEA, pH: 8.50+10% Ethylen glycol
Flow: 8 mL/min.

### Purification:

The reaction mixture was purified at an Äkta.

Diluted with 20 mM TEA, pH 8.50+10% ethylenglycol to a salt concentration at 0.1M NaCl.

### Column: HiLoad 26/10 Q Sepharose HP

### CIP: 1M NaOH

Buffer A: 20 mM Triethenolamine+10% Ethylen glycol pH 8.50
Buffer B: 20 mM Triethanolamine+10% Ethylen glycol pH 8.50+μM NaCl
Gradient 1: 0-15% Buffer B over 1 CV
Gradient 2: 15-25% buffer B over 10 CV
Gradient 3: 25-100% Buffer B over 1 CV
Flow: 5 mL/min

### Temp: RT

Fractions: 7 mL per fraction
Sample was applied using a sample pump with a flow at 5 mL/min:
Good separation with a narrow peak.
Fractions C10, C12, D9 and D7 were analyzed. All pure and containing the product.
Fraction from C10 through D7 were pooled.

### Volume: 63 mL

### Desalting:

The product was buffer changed to 10 mM ammoniumbicarbonate buffer at an Äkta.

### Column: HiPrep G25 Fine 50/20 Desalting

Buffer A: 10 mM ammoniumbicarbonate
Flow: 8 mL/min

### Run: 1.5 CV

### Temp: RT

Fractions: 45 mL per fraction
Sample was applied using a sample pump with a flow at 5 mL/min in one run:
Fractions A3 through A5 were pooled.
Total volume: 135 mL

The pool fractions were concentrated using Amicon Filter devises with a cut off at 10 kDa.

Centrifuged at 4000 rpm/6 min.

### Volume: 21.3 mL

### Quantified at HPLC:

Area found: 16991257, inj.: 5 μL, 2×FF, ex. coef.: 1000000=6.80 mg/mL×21.3 mL=144.5 mg

### Purity: 100%

HPLC: Rt=13.65 min.

LC-MS (electrospray): m/z: 22.994, 5 at Rt.: 6 min.

### Compound J

In a similar way as described in relation to compound E the following compound was prepared using Linker 13 and the GH compound hGH[Q84C, L101C, Y143C].

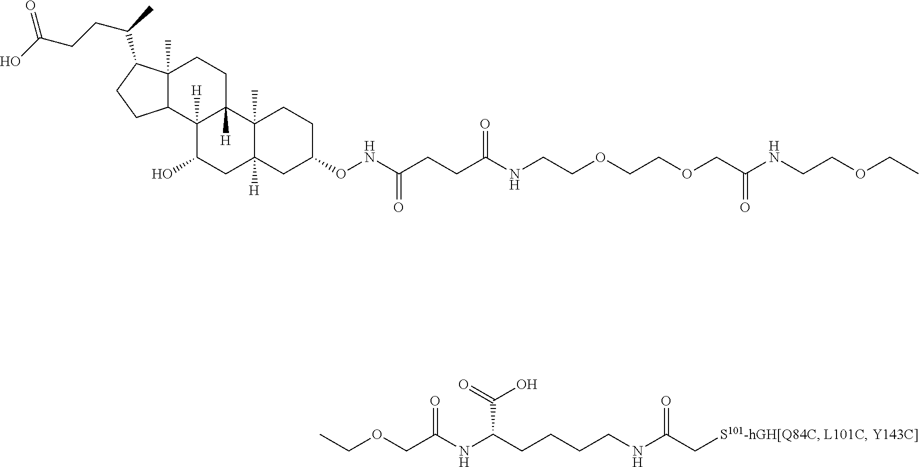

After coupling of protein and linker over night the reaction mixture was buffer changed to 20 mM triethenolamine+10% Ethylen glycol at pH 8.50.

### Buffer Change:

### Column: HiPrep 26/10 Desalting

Buffer A: 20 mM TEA, pH: 8.50+10% Ethylen glycol
Flow: 8 mL/min.

### Purification:

The reaction mixture was purified at an Äkta.

Diluted with 20 mM TEA, pH 8.50+10% ethylenglycol to a salt concentration at 0.1M NaCl.

### Column: HiLoad 26/10 Q Sepharose HP

### CIP: 1M NaOH

Buffer A: 20 mM Triethenolamine+10% Ethylen glycol pH 8.50
Buffer B: 20 mM Triethanolamine+10% Ethylen glycol pH 8.50+1M NaCl
Gradient 1: 0-14% Buffer B over 1 CV
Gradient 2: 14-25% buffer B over 10 CV
Gradient 3: 25-100% Buffer B over 2 CV
Flow: 5 mL/min

### Temp: RT

Fractions: 7 mL per fraction
Sample was applied using a sample pump with a flow at 5 mL/min:
Fractions C2, C11 and D8 checked TOF and fractions B1,C3, C6, C9, C12, D10 at HPLC,
Fraction from B1 through D5 were all pooled.

### Desalting.

The pooled fractions were buffer changed to 10 mM ammoniumbicarbonate buffer at an Äkta.

### Column: HiPrep G25 Fine 50/20 Desalting

Buffer A: 10 mM ammoniumbicarbonate
Flow: 8 mL/min

### Run: 1.3 CV

### Temp: RT

Fractions: 45 mL per fraction
Sample was applied using a sample pump with a flow at 5 mL/min. in 2 runs:

Fractions A4, A5 and A6 from the first run were pooled and fractions A3, A4 and A5 from the second run were pooled.

The pools were concentrated using Amicon Filter devises with a cut off at 10 kDa. Centrifuged at 4000 rpm/6 min.

### Volume: 30 mL

### Quantified at HPLC:

Area found: 17898813, inj.: 5 μL, 2×FF, ex. coef.: 1000000=3.58 mg/mL×29.5 mL=105.5 mg.

### Purity: 96% at Rt.: 13.63 min.

LC-MS (electrospray): m/z: 22.994

### Compound K

In a similar way as described in relation to compound E the following compound was prepared using Linker 5 and the GH compound hGH[L101C].

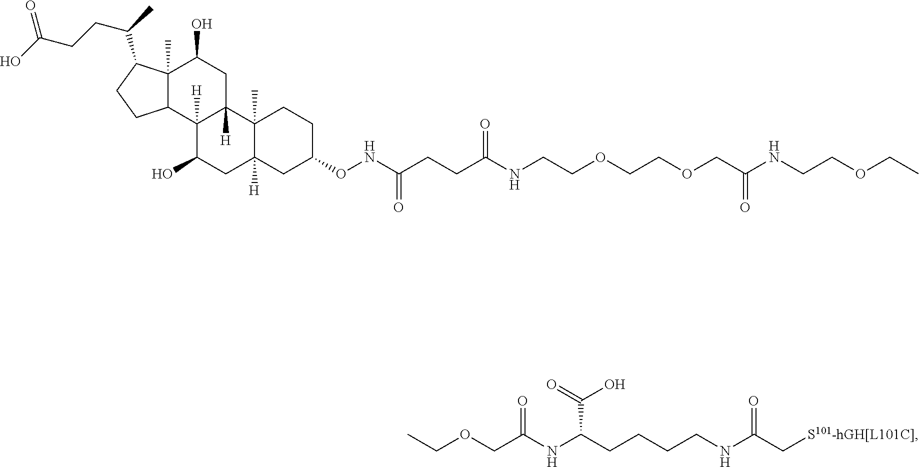

After coupling of protein and linker over night the reaction mixture was buffer changed to 20 mM triethenolamine+10% Ethylen glycol at pH 8.50.

### Buffer Chance:

### Column: HiPrep 26/10 Desalting

Buffer A: 20 mM TEA, pH: 8.50+10% Ethylen glycol
Flow: 8 mL/min.

### Purification No. 1:

The reaction mixture was purified at an Äkta.

Diluted with 20 mM TEA, pH 8.50+10% ethylenglycol to a salt concentration at 0.1M NaCl

### Column: HiLoad 26/10 Q Sepharose HP

### CIP: 1M NaOH

Buffer A: 20 mM Triethenolamine+10% Ethylen glycol pH 8.50
Buffer B: 20 mM Triethanolamine+10% Ethylen glycol pH 8.50+1M NaCl
Gradient 1: 0-15% Buffer B over 1 CV
Gradient 2: 15-30% buffer B over 10 CV
Gradient 3: 30-100% Buffer B over 2 CV
Flow: 5 mL/min.

### Temp: RT

Fractions: 7 mL per fraction
Sample was applied using a sample pump with a flow at 5 mL/min:
Fractions D5, D4, D1, E3 and E4 were analyzed at an HPLC:

In all fractions a peak just before the desired product was detected.

All fractions were pooled for a second purification using a different gradient.

### Purification No. 2:

The pooled fractions were purified at an Äkta.

Diluted with 20 mM TEA, pH 8.50+10% ethylenglycol to a salt concentration at 0.1M NaCl

### Column: HiLoad 26/10 Q Sepharose HP

Buffer A: 20 mM Triethenolamine+10% Ethylen glycol pH 8.50
Buffer B: 20 mM Triethanolamine+10% Ethylen glycol pH 8.50+μM NaCl
Gradient 1: 0-xx % Buffer B over 1 CV
Gradient 2: xx % buffer B over 10 CV
Gradient 3: xx-100% Buffer B over 2 CV
Flow: 5 mL/min.

### Temp: RT

Fractions: 7 mL per fraction
Sample was applied using a sample pump with a flow at 5 mL/min:

### Desalting.

The product was buffer changed to 10 mM Ammoniumbicarbonatebuffer at an Äkta.

### Column: HiPrep G25 Fine 50/20 Desalting

Buffer A: 10 mM ammoniumbicarbonate
Flow: 8 mL/min

### Run: 1.3 CV

### Temp: RT

Fractions: 45 mL per fraction
Sample was applied using a sample pump with a flow at 5 mL/min:

Fractions A3, A4 and A5 were pooled.

The pool fraction were concentrated using Amicon Filter devises with a cut off at 10 kDa. Centrifuged at 4000 rpm/6 min.

### Volume: 18 mL

### Quantified at an HPLC:

Area found: 11570188, inj.: 5 μL, 2×FF, ex. coef.: 1000000=2.31 mg/mL×2=4.62 mg/mL×17.7 mL=82 mg

### Rt.: 13.70 min.

LC-MS TOF: Mass found: 23.097 at Rt.: 6 min.

### Compound L

In a similar way as described in relation to compound E the following compound was prepared using Linker 5 and the GH compound hGH[Q40C, Q84C, Y143C].

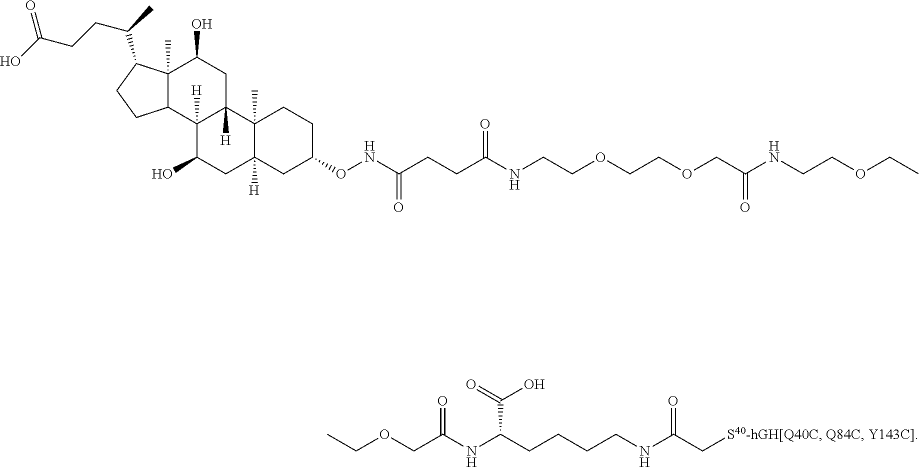

After coupling of protein and linker over night the reaction mixture was buffer changed to 20 mM triethenolamine+10% Ethylen glycol at pH 8.50.

### Buffer Chance:

### Column: HiPrep 26/10 Desalting

Buffer A: 20 mM TEA, pH: 8.50+10% Ethylen glycol
Flow: 8 mL/min.

### Purification:

The reaction mixture was purified at an Äkta.

Diluted with 20 mM TEA, pH 8.50+10% ethylenglycol to a salt concentration at 0.1M NaCl.

### Column: HiLoad 26/10 Q Sepharose HP

### CIP: 1M NaOH

Buffer A: 20 mM Triethenolamine+10% Ethylen glycol pH 8.50
Buffer B: 20 mM Triethanolamine+10% Ethylen glycol pH 8.50+μM NaCl
Gradient 1: 0-15% Buffer B over 1 CV
Gradient 2: 15-25% buffer B over 11 CV
Gradient 3: 25-100% Buffer B over 2 CV
Flow: 5 mL/min.

### Temp: RT

Fractions: 7 mL per fraction
Sample was applied using a sample pump with a flow at 5 mL/min.:

Fractions C2, C3, C5, C7, C9, C11, D12, D10, D8 and D6 were analyzed (HPLC). All pure, but two pools made and desalted separately.

### Pool I: C2 to C10

### Pool II: C11 to D8

### Desalting.

The two pools were buffer changed to 10 mM ammoniumbicarbonate buffer at an Äkta.

### Pool I.

### Column: HiPrep G25 Fine 50/20 Desalting

Buffer A: 10 mM ammoniumbicarbonate
Flow: 8 mL/min.

### Run: 1.3 CV

### Temp: RT

Fractions: 45 mL per fraction
Sample was applied using a sample pump with a flow at 5 mL/min.:

### Pool II.

### Column: HiPrep G25 Fine 50/20 Desalting

Buffer A: 10 mM ammoniumbicarbonate
Flow: 8 ml/min.

### Run: 1.3 CV

### Temp: RT

Fractions: 45 mL per fraction
Sample was applied using a sample pump with a flow at 5 mL/min.: Fractions A3, A4 and A5 from both runs were pooled.

The pools were concentrated using Amicon Filter devises with a cut off at 10 kDa.

Centrifuged at 4000 rpm/6 min.

### Volume: 31 mL

### Quantified by HPLC:

Pool 1, Area found: 8819533, inj.: 5 μL, ex. coff.: 1000000=1.76 mg/mL
Pool 2, Area found: 8974748, inj.: 5 μL, ex. coff.: 1000000=1.79 mg/mL
Both pools were mixed, affording a total volume of: 31 mL. Total: 55 mq

LC-MS (electrospray): m/z: 22.995, 6 at Rt.: 7 min.

HPLC: Rt=13.72 min.

### Example 1

### Analysis of Selected Compounds by BAF Assay and Proteolytic Digestion as Described Above (Method 5 and 8)

Growth hormone compounds comprising an additional disulphide bond were prepared and the in vitro activity and protease stability assayed. The results provided below were obtained after 4 hrs. (T=4).

TABLE 1
Proteolytic stability of GH compounds

| | EC50 BAF | | | Domains |
|---|---|---|---|---|
| | Ratio | Chymotrypsin | Elastase | linked by |
| | (hGH com- | stability Intact | stability Intact | additional |
| Compound | pound/hGH) | compound (%) | compound (%) | disulfide bond |
| GH | 1.0 | 42 | 25 | |
| hGH (A17C, E174C) | 0.6 | 45 | 10 | H1-H4 |
| hGH (H21C, M170C) | 0.5 | 72 | 10 | H1-H4 |
| hGH (D26C, V102C) | 0.5 | 55 | 65 | H1-L2 |
| hGH (D26C, Y103C) | 0.5 | 55 | 45 | H1-L2 |
| hGH (F54C, Y143C) | 0.6 | 55 | 20 | L1-L3 |
| hGH (F54C, S144C) | 0.5 | 60 | 20 | L1-L3 |
| hGH (F54C, F146C) | 0.6 | 40 | 25 | L1-L3 |
| hGH (S55C, Y143C) | 0.5 | 90 | 25 | L1-L3 |
| hGH (S57C, Y143C) | 0.3 | 75 | 50 | L1-L3 |
| hGH (I58C, Q141C) | 0.7 | 70 | 25 | L1-L3 |
| hGH (I58C, Y143C) | 0.6 | 55 | 20 | L1-L3 |
| hGH (I58C, S144C) | 1.2 | 65 | 30 | L1-L3 |
| hGH (P59C, Q137C) | 0.7 | 72 | 35 | L1-L3 |
| hGH (S71C, S132C) | 0.2 | 90 | 45 | L1-L3 |
| hGH (L81C, Y143C) | 0.7 | 85 | 15 | H2-L3 |
| hGH (Q84C, Y143C) | 0.5 | 100 | 80 | H2-L3 |
| hGH (S85C, Y143C) | 0.5 | 80 | 70 | H2-L3 |
| hGH (S85C, S144C) | 0.7 | 81 | 60 | H2-L3 |
| hGH (F92C, T148C) | 0.6 | 40 | 55 | H2-L3 |
| hGH (R94C, D107C) | 0.8 | 38 | 70 | H2-H3 |

### Example 2

### Analysis of Selected Compounds by BAF Assay and Proteolytic Digestion as Described in Method 5 and 8

Growth hormone compounds were tested according to Method 5 and 8

TABLE 2
Proteolytic stability of GH variant compounds

| | EC₅₀ BAF Ratio | Chymotrypsin | Elastase |
|---|---|---|---|
| | (hGH com- | stability Intact | stability Intact |
| Compound | pound/hGH) | compound (%) | compound (%) |
| HGH | 1.00 | 42 | 25 |
| HGH (Q84C, Y143C) | 0.66 | 90 | 80 |
| HGH (S62N) | 1.83 | 50 | 70 |
| HGH (S62Q) | 1.02 | 50 | 70 |
| HGH (Q84C, Y143C, S62T) | 0.29 | 80 | 50 |
| HGH (Q84C, Y143C, S62N) | 0.80 | 90 | 90 |
| HGH (Q84C, Y143C, S62C) | ND | 90 | 70 |
| HGH (Q84C, Y143C, S62Q) | 0.85 | 90 | 90 |
| HGH (Q84C, Y143C, S62H) | ND | 80 | 75 |
| HGH (Q84C, Y143C, S62E) | ND | 85 | 80 |
| HGH (Q84C, Y143C, L101C) | 0.99 | 100 | 75 |
| HGH (Q84C, Y143C, R134V) | 2.19 | 90 | 50 |
| HGH (Q84C, Y143C, G136V)) | 0.94 | 80 | 50 |
| HGH (Q84C, Y143C, T142N) | 0.55 | 80 | 65 |
| HGH (Q84C, Y143C, S144N) | 0.60 | 85 | 80 |
| HGH (Q84C, Y143C, S144N) | 0.44 | 100 | 75 |

### Example 3

### In Vitro Potency of Bile Acid Conjugates was Analyzed Using the BAF-3 hGH Receptor Assay (Method 5)

Protease stability of compounds of the present invention was determined as described in the general method by incubating the compound for 4 hrs. with chymotrypsin or elastase. The percent (%) of intact GH compound was measured and the results are included in the table below.

TABLE 3
Proteolytic stability of GH compounds.

| | | Ratio | Chymotrypsin | Elastase |
|---|---|---|---|---|
| | EC₅₀ | (EC₅₀ cmp/EC₅₀ | (% intact GH | (% intact GH |
| Compound | (nM) | hGH) | comp.) | comp.) |
| hGH | 0.026 | 1.00 | 40 | 25 |
| Compound A | 0.035 | 1.94 | 90 | 90 |
| Compound B | 0.017 | 0.81 | 95 | 90 |
| Compound C | 0.024 | 0.61 | 80 | 30 |
| Compound D | 0.028 | 0.62 | 95 | 65 |
| Compound E | 0.034 | 1.09 | 90 | 90 |
| Compound F | 0.054 | 2,.3 | 85 | 80 |
| Compound H | 0.016 | 1.34 | 80 | 65 |
| Compound I | 0.020 | 1.18 | 80 | 65 |
| Compound J | 0.009 | 1.17 | 70 | ND |
| Compound K | 0.011 | 1.03 | ND | ND |
| Compound L | 0.008 | 0.75 | 80 | 70 |

ND (not determined)

### Example 4

### Permeability of GH Compounds in Caco 2 Assay

The ability of GH compounds to be transported across monolayer of intestinal Caco-2 cells was determined as described in method 11 above. The apparent permeability (Pₐₚₚ) was measured in the absorptive direction and the Pₐₚₚ calculated for a series of GH compounds is listed in the table below.

| Compound | Pₐₚₚ (×10⁻⁸ cm/s) |
|---|---|
| hGH (Q84C, Y143C) | 0.24 ± 0.07 |
| E | 1.2 ± 0.87 |
| F | 9.0 ± 8.40 |
| H | 0.8 ± 1.10 |
| I | 0.4 ± 0.22 |
| J | 0.34 ± 0.06 |
| K | 0.3 ± 0.03 |
| L | 1.0 ± 0.11 |

All values are mean ± SD, n = 4

The Pₐₚₚ calculated demonstrates that the protease stabilised GH compounds and/or cholic acid conjugated GH compounds are transported across the Caco-2 cell monolayer. For some compounds a high Pₐₚₚ is obtained, whereas other compounds displays low transport with Pₐₚₚ.values below 0.5×10⁻⁸ cm/s.

### Example 5

### Pharmacokinetic Properties of GH Compounds

Pharmacokinetic properties of hGH, hGH (Q84C, Y143C) and hGH (Q84C, Y143C, L101C) with a cholic acid derivative in mini pigs were investigated after endoscope and i.v. administration. hGH, hGH (Q84C, Y143C) and hGH (Q84C, Y143C, L101C) with a cholic acid derivative (Compound E) were analysed following the above described procedure (Method 9 herein). The calculated pharmacokinetic parameters are included in the table below.

TABLE 54A
Pharmacokinetic properties of growth
hormone compounds and conjugates

| | | | | AUC (0-8 |
|---|---|---|---|---|
| | Administration | Dose | t½ | hr) (hr*nmol/ |
| Compound | route | (nmol/kg) | (hr) | L/nmol) |
| hGH | i.v. | 20 | 0.6 | |
| hGH (Q84C, | i.v. | 20 | 1.2 | |
| Y143C) | | | | |
| hGH (Q84C, | i.v. | 20 | 1.7 | |
| Y143C, | | | | |
| L101C) with | | | | |
| cholic acid | | | | |
| conjugation | | | | |
| (Compound E) | | | | |
| hGH | Intra- | 800 | | 0.072 |
| | intestinal | | | |
| hGH (Q84C, | Intra- | 800 | | 0.433 |
| Y143C) | intestinal | | | |
| hGH (Q84C, | Intra- | 800 | | 2.675 |
| Y143C, | intestinal | | | |
| L101C) with | | | | |
| cholicacid | | | | |
| conjugation | | | | |
| (Compound E) | | | | |

In a further study the pharmacokinetic properties of hGH, hGH (Q84C, Y143C) and hGH (Q84C, Y143C, L101C) with a cholic acid derivative in Sprague Dawley rats were investigated after intra-intestinal injection as described above. hGH (Q84C, Y143C) and hGH (Q84C, Y143C, L101C) with a cholic acid derivative (Compound E) was analysed following the above described procedure (Method 10 herein). The calculated pharmacokinetic parameters are included in the table below.

TABLE 5B
Pharmacokinetic properties of growth
hormone compounds and conjugates

| | | | | AUC (0-4 |
|---|---|---|---|---|
| | Administration | Dose | t½ | hr) (hr*nmol/ |
| Compound | route | (nmol) | (hr) | L/nmol) |
| hGH | Intra- | 300 | 1.1 | 0.372 |
| | intestinal | | | |
| hGH (Q84C, | Intra- | 300 | 1.1 | 15.4 |
| Y143C) | intestinal | | | |
| hGH (Q84C, | Intra- | 300 | 1.4 | 33.1 |
| Y143C, | intestinal | | | |
| L101C) with | | | | |
| cholic acid | | | | |
| conjugation | | | | |
| (Compound E) | | | | |

1. A growth hormone conjugate of formula (I)
A-W—B-GH (I)
wherein
GH represents a growth hormone compound,
A represents a bile acid residue,
B represents a hydrophilic spacer covalently linked to GH,
W is a chemical group linking A and B;
and a pharmaceutically acceptable salt.

2. The growth hormone conjugate according to claim 1, wherein the growth hormone compound GH comprises one or more additional disulfide bond(s) compared to human growth hormone (hGH) as defined by SEQ ID NO 1.

3. The growth hormone conjugate according to claim 2, wherein the growth hormone compound comprises one or more additional disulfide bond(s) connecting a loop segment and a helical segment.

4. The growth hormone conjugate according to claim 1, wherein the compound comprise at least one pair of mutations corresponding to R16C/L117C, A17C/E174C, H21C/M170C, D26N102C, D26/Y103C, N47C/T50C, Q49C/G161C, F54C/Y143C, F54C/S144C, F54C/F146C, S55C/Y143C, S57C/Y143C, 158C/Q141C, 158C/Y143C, 158C/S144C, P59C/Q137C, P61C/E66C, P61C/T67C, S71C/S132C, L73C/S132C, L73C/F139C, R77C/1138C, R77C/F139C, L81C/Q141C, L81C/Y143C, Q84C/Y143C, Q84C/S144C, S85C/Y143C, S85C/S144C, P89C/F146C, F92C/F146C, F92C/T148C, R94C/D107C, V102C/A105C, L156C/F146C, L156C/T148C and/or V185C/S188C in SEQ ID NO 1.

5. The growth hormone compound according to claim 2, wherein the growth hormone compound comprises one or more additional disulfide bond(s) wherein at least one of the cysteines thereof is present in loop 3 (L3) corresponding to AA 128-154 in SEQ ID NO 1.

6. The growth hormone conjugate according to claim 5, wherein the growth hormone compound comprises one or more additional disulfide bond connecting L3 with helix 2 (H2) or loop 1 (L1).

7. The growth hormone conjugate according to claim 1, wherein the bile acid residue (A) is a cholic acid residue.

8. The growth hormone conjugate of claim 7, wherein the cholic acid residue is selected from 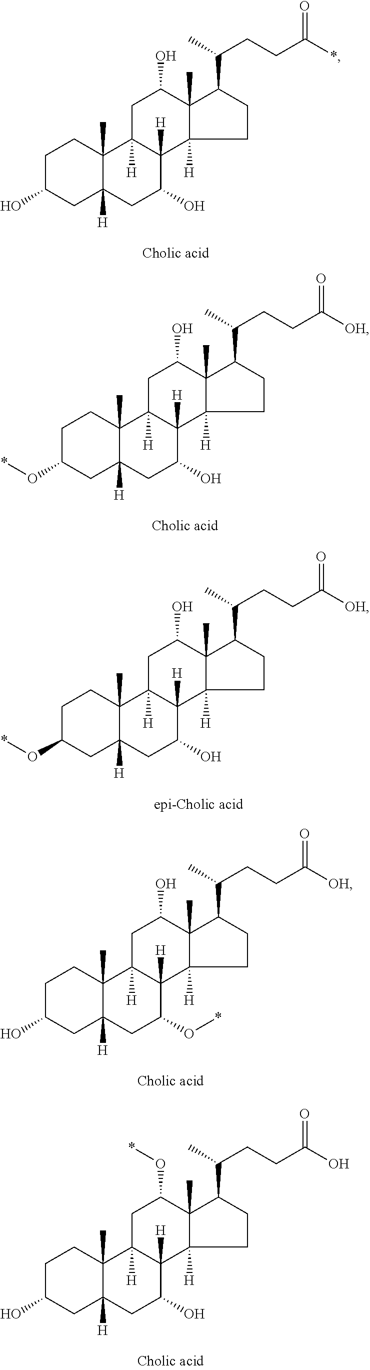
and wherein (*) represents the attachment point to the hydrophilic spacer (B) through W.

9. The growth hormone conjugate according to claim 1, wherein GH is conjugated at a wild type amino acid residue selected from the group of: the N-terminal, Gln40 and Gln141.

10. The growth hormone conjugate according to claim 1, wherein GH is conjugated at a mutant amino acid residue.

11. The growth hormone conjugate according to claim 10, wherein the mutant amino acid residue is a cysteine (Cys).

12. The growth hormone conjugate according to claim 11, wherein the cys mutant is in a position corresponding to position 101 in human growth hormone.

13. A method for preparing a growth hormone conjugate according to claim 1, comprising
a) conjugating a cholic acid linker to a growth hormone compound (GH)
b) obtaining said growth hormone conjugate.

14. A pharmaceutical composition comprising growth hormone conjugate according to claim 1 and a pharmaceutically acceptable carrier.

15. A method of treating a disease or a state in a patient wherein the disease or the state may be treated by growth hormone activity and the patient will benefit from an increase in the amount of circulating growth hormone, said method comprising administering to the patient an effective amount of a growth hormone conjugate according to claim 1.

16. A method of treating a disease or a state in a patient wherein the disease or the state may be treated by growth hormone activity and the patient will benefit from an increase in the amount of circulating growth hormone, said method comprising administering to the patient an effective amount of a pharmaceutical composition according to claim 14.
